SEQUENCE LISTING (91 sequences)

SEQ ID NO: 1 - DNA, 58 bp, Artificial Sequence
  Synthetic oligonucleotide
aatttttttttataaatacggaagaaaatatacgagctaaattttatgttcttccgtt

SEQ ID NO: 2 - DNA, 36 bp, Artificial Sequence
  Synthetic oligonucleotide
tatggggcggccgcctttattataacataatgaatg

SEQ ID NO: 3 - DNA, 3179 bp, Artificial Sequence
  Vector sequence
ggccgctccctggccgacttggcccaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggc

SEQ ID NO: 4 - DNA, 2614 bp, Dunaliella salina
ggccgcctttattataacataatgaatgactaatgtcaattgtttatttgaaaattaacttcaataaaaatttacaaagagaaaaaaattaaccggatttttctttgataaaaatacgtaggaaacaatattttattttgtttataacaaaaaaaagtttaaaatgaaaaaatcacgtttataccgaatttaaacgtttactattaatactaatgaatttaatgtactaataagaagagttatataactattcaaattaacaaaaagttaaaaggaaacctcctgtgttttaattaaaacacaggaggtttatctcatttacttgataacaaaatattaaagaagtgatatttctatctgggtttcaaacgcaagggcctcttagagaggaacactttaaattatataaatttatttagcggctaaactttcccagctattagtaacaccatctaaaattaatgaactattataaatttctagaataataagtaaaaaaaccgcaaataaaagaattgctacagccataagaactgtagtaccccatccaggtaaaactttacctgcttcagagtttagaggacgtaataaagttcctaatggtgtaacaattcctggttcttgtgatgttgaagtttgtgtactattttttcctgtagccataattgatagttaataaaatctttttgtttttttcctttctgtaatattgtataatatatatggagaataattttgtcttgtcaaaaattttaaatttatggaaagtccggcttttttctttaccttctttttatggtttcttttattaagtgctacaggttattcagtttatgttagttttggacctccttcaagaaaattgagagatccttttgaagaacatgaagattaaattaataatcttagttaagtaaaaattttaagtattctaagggttggacttcactaattaatgttaatgaaatccaacccttataatacttcatttgaaacgtatttacgataaatatagaatttctcgtagattttcgtatcggaaaaaacaactttattgtttggtccgacaagtaattttaataaaaaattattctattactattttgcaatacgtggaggctctctaaaaaagatagagaaaaagataatacctaacgttccaattaataagaaagtgtaaactaaagcttccatgaaaggtgtttaataaatttattgaaaagactagtcttttcaaataggaacataataccaaattttacattagtgtaaaacaaaaagaattttcttccgaattacgaaaagaaaataaacgaagcggtcagaagataaatttaaaatatctaacgacttacctaaagttataaaagataaaatttaattccaataaggagttaaaaaaaatattatcttagatttttttaacaaaaataaaatattaacattttataaaaataaaacggaagaacataaaatttagcgtttaaacgaattcgcccttcccgggatcctaggtcgtatattttcttccgtatttataaaaaaaaattctttttatgaaataaactttgatcaaatttgtttacactaactcaaattcttttgctcagagaaaatctaagcccatctaaaaaaaaaaaaacaattataccgtattaaaatctacggtaagatagaaaatctaataaagataagaaaaatcacattacaaaaaaatcacattacaaaatatgtgaactttgttaaatgaatcttctattttctagtcggaaaacaaaaaaacaaagaaaagtgtttagtccgccaaaaagagaaaaaatctattagaatttctcgacggaaattctaatagattttttctatatgaatttaaaaacaagaatttctaaatattcttggtagaatattggaataaaacttaatatagtgattagaaagcttcacgaacagatgaagtatcaccaagtttcttatatttaccgaattctaattgatcattaatgtcttcatcaataccagcgaaaacgtcacggaaaatagttcttgaaccatgccaaatatgaccaaagaagaataataaggcaaaagataagtgtccaaaagtgaaccaaccacgtgggctactacggaatacaccgtcagattgtaaagtcgaacggtcaaattcaaagatttcacctaattgagctttacgtgcatattttttaacagttgaagggtcagtaaatgttaaaccatttaattcaccaccatagaatgtaactgaaacaccaacttgttcaattgagtattttgattcagctttacggaatggtacgtcagcacgaacaacaccgtctttatcaattaaaacaacagggaaagtttcaaagaaagtaggcatacgacgaacaaaaagttcacgaccttcttgatctttaaaactagcgtgtcctaaccaacctacagcgataccatcaccactgttcatagcacctgtacggaataatccacctttagctgggttattaccaatgtaatcatagaaagctaatttttcaggaatttttgcccaagcttctgaaacagataaaccttcagatgtactttgtgctactcgtttttgaatttcttg

SEQ ID NO: 5 - DNA, 47 bp, Artificial Sequence
  Synthetic oligonucleotide
tattaatcctaggatcccgggttatatatagttaatttttataaaag

SEQ ID NO: 6 - DNA, 63 bp, Artificial Sequence
  Synthetic oligonucleotide
taaacccgtttaaacttgcatgcctcgaggatatcaccatggtattatctaaaaatgaaacat

SEQ ID NO: 7 - DNA, 44 bp, Artificial Sequence
  Synthetic oligonucleotide
tgatatcctcgaggcatgcttttttcttttaggcgggtccgaag

SEQ ID NO: 8 - DNA, 37 bp, Artificial Sequence
  Synthetic oligonucleotide
ttcgtctagtttaaacttagcgcagcggacagacaac

SEQ ID NO: 9 - DNA, 248 bp, Dunaliella salina
gatcccgggttatatatagttaatttttataaaagaaaattaaacaaataaagcataataagttattataaatacaggaacgaaattatatagaattataatttataaattggaaattagaaaaaaattatatgttctttaattaccaaaatttaaatttggtaaaagattattatatcatcggatagattattttaggatcgacaaaaatgtttcatttttagataataccatggtgatatcctcga

SEQ ID NO: 10 - DNA, 430 bp, Dunaliella salina
ggcatgcttttttcttttaggcgggtccgaagtccttaggcttattcgaaggaaaaacgagaaaaatttacgtagtaaattttctttgctggccctgccaaaaacaacaccattaacctataagtagtaataattctttagtattacttttaggttatttataaatttgagaagtatagaagaatctatagattttgcttatgtgtttatctatagattcttctatacttctcatttttaacaaatttttattaagatttttttaaacaaaaaaaaagttttcaacttatataattaaacctaaacaacgttgtatattttttattttaagttttggtaaagtatgtataccagtaaacctttagtaaatttttttaccgcttaggctaggacctataaaatttagcgcggcgcaagggcgaattcgttt

SEQ ID NO: 11 - DNA, 47 bp, Artificial Sequence
  Synthetic oligonucleotide
acgttattaatcctaggatcccgggcactcaaaagataggacgacga

SEQ ID NO: 12 - DNA, 53 bp, Dunaliella salina
gtttaaacttgcatgcctcgaggatatcaccatggcctttaagtagaggatgc

SEQ ID NO: 13 - DNA, 683 bp, Dunaliella salina
cactcaaaagataggacgacgattaagaaaaaacaatatatatatgccaattggtgttccacgtattatttatagttggggtgaagaacttccagctcaatggactgatatttataattttattttccgtcgaagaatggtttttttaatgcaatatttagatgacgaactttgtaaccaaatttgtggtttattaattaatatccatatggaagatcgatctaaagaacttgaaaaaaacgaagtcgaaggagattcaaaacctcgttcaactagtagtgaaaagagaactgatggtccatcttctgtgaagaaaaatagatctcctgaagatttattaaatgctgatgaagatttaggtattgatgatattgatacattagaacaattaacattacaaaaaattacaaaagaatggctaaattggaattcacagttttttgattattcagatgaaccttatttatattatttagcacaaactttatcaaaagattttggtaatagcwmttctmgtysgccttrcgatwttmryscwcacaattttttaatagtttaaaaagtaattccttaaacttacaaaatagaaaaagtgcaccttctggtaaaggactagatatttattcagcatttagaacaagtttaaattttgaaaatgaaggtgcgggtgcatatagcttaaa

SEQ ID NO: 14 - DNA, 47 bp, Artificial Sequence; identical to SEQ ID NO: 11
  Synthetic oligonucleotide

SEQ ID NO: 15 - DNA, 51 bp, Artificial Sequence
  Synthetic oligonucleotide
aaacttgcatgcctcgaggatatcaccatggcctttaagtagaggatgcat

SEQ ID NO: 16 - DNA, 744 bp, Dunaliella salina
gatcccgggcactcaaaagataggacgacgacactcaaaagataggacgacgattaagaaaaaacaatatatatatgccaattggtgttccacgtattatttatagttggggtgaagaacttccagctcaatggactgatatttataattttattttccgtcgaagaatggtttttttaatgcaatatttagatgacgaactttgtaaccaaatttgtggtttattaattaatatccatatggaagatcgatctaaagaacttgaaaaaaacgaagtcgaaggagattcaaaacctcgttcaactagtagtgaaaagagaactgatggtccatcttctgtgaagaaaaatagatctcctgaagatttattaaatgctgatgaagatttaggtattgatgatattgatacattagaacaattaacattacaaaaaattacaaaagaatggctaaattggaattcacagttttttgattattcagatgaaccttatttatattatttagcacaaactttatcaaaagattttggtaatagcwmttctmgtysgccttrcgatwttmryscwcacaattttttaatagtttaaaaagtaattccttaaacttacaaaatagaaaaagtgcaccttctggtaaaggactagatatttattcagcatttagaacaagtttaaattttgaaaatgaaggtgcgggtgcatatagcttaaaatgcatcctctacttaaaggccatggtgat

SEQ ID NO: 17 - DNA, 41 bp, Artificial Sequence
  Synthetic oligonucleotide
ccgccgggcggatccctgtaagtttctttcaaaaatacatg

SEQ ID NO: 18 - DNA, 39 bp, Artificial Sequence
  Synthetic oligonucleotide
gtcccgaagtcctgcagtgcgtgcatctccataataatt

SEQ ID NO: 19 - DNA, 710 bp, Dunaliella salina
atactaggatccgtttaaacctgcagatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaaagcttctcgagggtacccacgtg

SEQ ID NO: 20 - DNA, 1373 bp, Dunaliella salina
ccgccgggcggatccctgtaagtttctttcaaaaatacatgtccatttttttataaacaaacgggaggggtcgtctcataaaaaggaaatttttcttaaacaattttagcgaagcggtcagagaaaattatattagaatttctcgaagattttcaatatctcaaagagcaggaccgattgaaaacttcgatattttctaaaactcttttgacttttcgtgagataaaataaaagagatacagtcaataataaatttaacttgattaaatttattcttttccgttcttgtttttttctaatttacagtattaaaacagaaaaaaagtaaggctaaatatcttaaggaaatataaaacacaattgtttttttcaaatttttggttttttgaaaaattaaacaaataaaagcagtaaaacgtagaaaatatagaagttctaaataccaggagataaaccctttgggtttatctttttgctgcactaattaaaaaacgattttataatcatatagaatccgattaagatagtttgatttgttattgtttcattaatttttaattgataacttgcattagtttataactatcggatttttccttaagaaaaatccgtaggaaaaaatcttttaaaatattttttgtaagaaaaatcaatctatcagattacaattttatttcaagcctatctttttattaattcaattcaaacgaggatgttctctattgagaattaggattcttttcaagacttaatacatatacttttacttattgtattattaataataatggttttattaaaaaaaattataatatctactaaacatttaacattaggcgggttcgttaacctttaaggttaaagagatatatgttaaattaaacataaacgaaaagactttaaatttttcaaataaaaaaaaagatacagagggtactaatatttaatattatgaccttctgtatcctatacttaataagtataaattataatatagattaataaatctattcaagttaataaactgtgtttttattttatttaatgattttctctactaaatattaaatatgttattatttatacatagtgttttttcttttttttttttaagcctgtttaactcaatcggtagagtattggttttgtaaaccaaaggttgcgggttcgattcctgtagcaggctactaattttttaagatattttatattttaaaaatatctttttaaaataaaaaaaaaattttttaaatcgattttaaaaataaaaaaagctatacttataaatgcaataaaggttaaaaaaaaaattaaacgatatgatgaattataaaaattattatggagatgcacgcactgcaggacttcgggac

SEQ ID NO: 21 - DNA, 36 bp, Artificial Sequence
  Synthetic oligonucleotide
catttttagataataccatggaattaccaaatatta

SEQ ID NO: 22 - DNA, 60 bp, Artificial Sequence
  Synthetic oligonucleotide
gcatgcctgcagagtattttagataatgcttggaatcaattcaattcatcaagttttaaa

SEQ ID NO: 23 - DNA, 809 bp, Escherichia coli
ccatggctcgtgaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgcaggcatgc

SEQ ID NO: 24 - DNA, 811 bp, Escherichia coli
ccatggaattaccaaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaattcatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagatcttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattgattccaagcattatctaaaatactctgcag

SEQ ID NO: 25 - DNA, 674 bp, Escherichia coli
ccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaaagcttctcgag

SEQ ID NO: 26 - DNA, 62 bp, Artificial Sequence
  Synthetic oligonucleotide
tttatagagcatgcgattcccattaggaggtagtaccaaatggccgaggagatgatccccgc

SEQ ID NO: 27 - DNA, 44 bp, Artificial Sequence
  Synthetic oligonucleotide
gcgcgccgcatgcgagctctcaggccgtcaccggcggaaagatc

SEQ ID NO: 28 - DNA, 574 bp, Rhodobacter capsulatus
gcatgcgattcccattaggaggtagtaccaaatggccgaggagatgatccccgcctgggtcgagggcgtgctgcaacccgtcgagaagctggaggcccaccgcaagggcctgcggcatctggcgatttcggtcttcgtgacgcgcggcaacaaggtgcttttgcagcaacgcgcgctgtcgaaatatcacacgccggggctttgggcgaatacctgctgcacccatccctattggggcgaggatgcgccgacctgcgccgcccgccgtctggggcaggagctgggcatcgtcgggctgaagctgcgccacatggggcagctggaataccgcgccgatgtgaacaacggcatgatcgagcatgaggtggtggaggtcttcaccgccgaagcgcccgaggggatcgagccgcaacccgaccccgaggaagtggccgataccgaatgggtgcgcatcgacgcgctgcgctcggagatccacgccaatccggaacgcttcacgccctggctcaagatctatatcgagcagcaccgcgacatgatctttccgccggtgacggcctgagagctcgcatgc

SEQ ID NO: 29 - DNA, 50 bp, Artificial Sequence
  Synthetic oligonucleotide
caaattgcatgcggaggactacttattatgtcaattctttcttggatcga

SEQ ID NO: 30 - DNA, 46 bp, Artificial Sequence
  Synthetic oligonucleotide
taggtagcatgcattagctaaaattttggtctaattcgaaattctg

SEQ ID NO: 31 - DNA, 1280 bp, Chlorella vulgaris
caaattgcatgcggaggactacttattatgtcaattctttcttggatcgaaaatcaacgaaaattgaaattattaaatgcacctaaatacaatcatccagagtcagacgtaagtcaaggtctttggacacgctgcgaccattgtggtgtaatattatatattaaacatttaaaagaaaaccaacgtgtatgttttggttgcggatatcatctacaaatgagtagtacagaacgaattgagtcactagttgatgcaaatacgtggcgtccctttgatgaaatggtgtcaccatgtgatccattagaatttcgagatcaaaaagcctatacagaaagattaaaagacgcacaagaacgaacaggtctgcaagatgctgttcaaacaggaacaggacttcttgacggtattccgatagccttaggagttatggattttcattttatggggggaagtatgggctctgtagttggtgaaaaaatcacgcgtttaatagaatacgcaactcaagaaggtttacccgtaattttagtttgtgcttctggcggagctcgaatgcaagaaggtattttaagcttaatgcaaatggcaaaaatttctgccgctcttcatattcaccaaaattgcgccaaattactttatatttcagtcttaacttcaccaacaacaggtggtgtaactgctagctttgctatgttaggggatcttctttttgcagaaccaaaagctttaattgggtttgctggtcgtcgggtgattgaacaaaccttacaagagcaattacctgatgattttcaaactgctgagtatttgttacatcatggtcttcttgatttaatcgtaccacgatcttttttaaaacaagctttatctgaaaccctaacactttataaagaagctccgttaaaagaacagggtcggattccttatggtgaacgtgggcctcttacaaaaactcgtgaagaacaacttcgtcggtttcttaaatcgtcaaaaactcctgaatatttacatattgtaaatgatttaaaagaattacttggttttttaggtcaaactcagaccactctttaccctgaaaaactggaatttttaaataacctaaaaacccaagaacagtttctacaaaaaaatgataatttttttgaagagcttttaacttcaacaacagtaaaaaaagctttgaatttagcttgtggaacacaaacccgtctgaattggcttaattataagttaacagaatttcgaattagaccaaaattttagctaatgcatgctaccta

SEQ ID NO: 32 - DNA, 64 bp, Artificial Sequence
  Synthetic oligonucleotide
ctttatagactcgagaggaggaaaaaagtacatgttgcctgactggagcatgctctttgcagtg

SEQ ID NO: 33 - DNA, 46 bp, Artificial Sequence
  Synthetic oligonucleotide
gcgcgccctcgagttacaccctcggttctgcgggtatcacactaat

SEQ ID NO: 34 - protein, 10 aa, Unknown
  Conserved motif
YPTAWGDTVV

SEQ ID NO: 35 - protein, 10 aa, Unknown
  Conserved motif
WNDLDVNQHV

SEQ ID NO: 36 - protein, 6 aa, Unknown
  Conserved motif
EYRREC

SEQ ID NO: 37 - DNA, 24 bp, Artificial Sequence
  Synthetic oligonucleotide
tanccnncntggggngannnngtn

SEQ ID NO: 38 - DNA, 30 bp, Artificial Sequence
  Synthetic oligonucleotide
acntgntgnttnacntcnanntcnttccan

SEQ ID NO: 39 - DNA, 30 bp, Artificial Sequence
  Synthetic oligonucleotide
tggaanganntngangtnaancancangtn

SEQ ID NO: 40 - DNA, 18 bp, Synthetic oligonucleotide
  misc_feature 3, 6, 8, 9, 11, 12, 15, 18: n = A,T,C, G or inosine
cantcncnncnntantcn

SEQ ID NO: 41 - DNA, 24 bp, Artificial Sequence
  Synthetic oligonucleotide
tanccnncntggggngannnngtn

SEQ ID NO: 42 - DNA, 18 bp, Artificial Sequence
  Synthetic oligonucleotide
cantcncnncnntantcn

SEQ ID NO: 43 - DNA, 1019 bp, Umbellularia californica
ctttatagactcgagaggaggaaaaaagtacatgttgcctgactggagcatgctctttgcagtgatcacaaccatcttttcggctgctgagaagcagtggaccaatctagagtggaagccgaagccgaagctaccccagttgcttgatgaccattttggactgcatgggttagttttcaggcgcacctttgccatcagatcttatgaggtgggacctgaccgctccacatctatactggctgttatgaatcacatgcaggaggctacacttaatcatgcgaagagtgtgggaattctaggagatggattcgggacgacgctagagatgagtaagagagatctgatgtgggttgtgagacgcacgcatgttgctgtggaacggtaccctacttggggtgatactgtagaagtagagtgctggattggtgcatctggaaataatggcatgcgacgtgatttccttgtccgggactgcaaaacaggcgaaattcttacaagatgtaccagcctttcggtgctgatgaatacaaggacaaggaggttgtccacaatccctgacgaagttagaggggagatagggcctgcattcattgataatgtggctgtcaaggacgatgaaattaagaaactacagaagctcaatgacagcactgcagattacatccaaggaggtttgactcctcgatggaatgatttggatgtcaatcagcatgtgaacaacctcaaatacgttgcctgggtttttgagaccgtcccagactccatctttgagagtcatcatatttccagcttcactcttgaatacaggagagagtgcacgagggatagcgtgctgcggtccctgaccactgtctctggtggctcgtcggaggctgggttagtgtgcgatcacttgctccagcttgaaggtgggtctgaggtattgagggcaagaacagagtggaggcctaagcttaccgatagtttcagagggattagtgtgatacccgcagaaccgagggtgtaactcgagggcgcgc

SEQ ID NO: 44 - protein, 10 aa, Unknown
  Conserved motif
GDTQRFINIC

SEQ ID NO: 45 - protein, 13 aa, Unknown
  Conserved motif
KKDIVKLQHGEYV

SEQ ID NO: 46 - protein, 10 aa, Unknown
  Conserved motif
EKFEIPAKIK

SEQ ID NO: 47 - DNA, 31 bp, Artificial Sequence
  Synthetic oligonucleotide
ggnganacncanngnttnatnaanatntgnn

SEQ ID NO: 48 - DNA, 33 bp, Artificial Sequence
  Synthetic oligonucleotide
acntantcntgntgnannacnatntcnttnttn

SEQ ID NO: 49 - DNA, 33 bp, Artificial Sequence
  Synthetic oligonucleotide
aanaanganatngtnntncancangantangtn

SEQ ID NO: 50 - DNA, 27 bp, Artificial Sequence
  Synthetic oligonucleotide
ttnatnttnggnatntcnaanttntcn

SEQ ID NO: 51 - DNA, 31 bp, Artificial Sequence; identical to SEQ ID NO: 47
  Synthetic oligonucleotide

SEQ ID NO: 52 - DNA, 27 bp, Artificial Sequence
  Synthetic oligonucleotide
ttnatnttnggnatntcnaanttntcn

SEQ ID NO: 53 - DNA, 2076 bp, Arabidopsis thaliana
atgattccttatgctgctggtgttattgtgccattggctttgacgtttctggttcagaaatctaagaaagaaaagaaaagaggtgttgttgttgatgttggtggtgaaccaggttatgctattaggaatcacaggtttactgagcctgttagttcccattgggaacatatctcaacgcttccagagctctttgagatatcgtgtaatgctcacagtgatagggttttccttggcacccgaaagctgatctctagagagattgagactagtgaggatggaaaaacgttcgagaaactgcatttaggtgactacgagtggctcacttttgggaagactctcgaagcagtgtgtgattttgcctctgggttagttcagattgggcacaagacggaagagcgtgtcgccatttttgcagatactagagaagaatggttcatctccctacagggttgcttcaggcgcaacgtcactgtggtaactatctattcatctttgggagaggaagctctttgtcactcgctgaatgagacagaggtcacaaccgtaatatgtggtagcaaagaactcaaaaagctcatggacataagccaacagcttgaaactgtgaaacgtgtgatatgcatggatgatgaattcccatctgatgtgaacagtaattggatggcgacttcatttactgatgttcagaaacttggccgcgaaaatcctgtggatcctaatttccctctctcagcagatgttgctgttataatgtacaccagtggaagcactggacttcccaagggtgttatgatgacgcatggtaatgtcctagctacagtttcggcagtgatgacaattgttcctgaccttggaaagagggatatatacatggcatatttacctttggctcacatccttgagttagcagctgagagcgtaatggctactattgggagtgctattggatatgggtctcccttgacgctaacggatacttcaaacaagataaaaaagggtacaaaaggagatgtcacagcactaaagcccactataatgacagctgttccagccattcttgatcgtgtcagggatggtgtccgcaaaaaggttgatgcaaagggcggattgtcaaagaaattgtttgactttgcatatgctcggcgattatctgcaatcaatggaagttggtttggagcctggggattggaaaagcttttgtgggatgtgcttgtgttcaggaaaatccgtgcagttttgggaggtcaaatccgctatttgctctctggtggtgcccctctttctggtgacactcagagattcattaacatctgcgttggggctccaatcggtcagggatatgggctcacagagacttgtgctggtggaaccttctcggagtttgaggacacatccgttggccgtgttggtgctccacttccttgctcctttgtaaagctagtagactgggcggaaggtgggtatctaactagtgataagccgatgccccgtggtgaaattgtaattggtggctcaaatatcacgcttgggtatttcaaaaatgaggagaaaactaaagaagtgtacaaggttgatgaaaagggaatgaggtggttctacacaggagacataggacgatttcaccctgatggctgcctcgagataatagaccgaaaaaaggatatcgttaaacttcagcatggagaatatgtctccttgggcaaagttgaagctgctctaagtataagtccctatgttgaaaacataatggttcatgctgattcgttctacagttactgtgtggctcttgtggtcgcgtcccaacatacagttgaaggttgggcttcaaagcaaggaatagactttgccaacttcgaagaactgtgcacgaaagagcaagccgtgaaagaagtgtatgcgtcccttgtgaaggcggctaaacaatcacgattggagaagtttgagataccagcaaagatcaaattattggcatctccatggacgccagagtcaggattagtcacagcagctctaaagctgaaaagagatgtaattaggagggaattctctgaagatctcaccaagttatatgcctaa

SEQ ID NO: 54 - protein, 8 aa, Unknown
  Conserved motif
GKMFGFVH

SEQ ID NO: 55 - protein, 11 aa, Unknown
  Conserved motif
EGIPVATGAAF

SEQ ID NO: 56 - DNA, 25 bp, Artificial Sequence
  Synethetic oligonucleotide
ggnaaratgttnggnttngtncann

SEQ ID NO: 57 - DNA, 27 bp, Artificial Sequence
  Synethetic oligonucleotide
aangcngcnccngtngcnacnggnatn

SEQ ID NO: 58 - DNA, 1717 bp, Arabidopsis thaliana
aacctcgtcttctccgtccacttcactctctctaaactctctctcagatctctctctctctgtgattcaacaatggcggtttcttcttcttcgtttctatcgacagcttcactaaccaattccaaatccaacatttcattcgcttcctcagtatccccatccctccgcagcgtcgttttccgctccacgactccggcgacttctcaccgtcgttcaatgacggtccgatctaagattcgtgaaattttcatgccggcgttatcatcaaccatgacggaaggcaaaatcgtgtcatggatcaaaacagaaggcgagaaactcgccaagggagagagtgttgtggttgttgaatctgataaagccgatatggatgtagaaacgttttacgatggttatcttgctgcgattgtcgtcggagaaggtgaaacagctccggttggtgctgcgattggattgttagctgagactgaagctgagatcgaagaagctaagagtaaagccgcttcgaaatcttcttcttctgtggctgaggctgtcgttccatctcctcctccggttacttcttctcctgctccggcgattgctcaaccggctccggtgacggcagtatcagatggtccgaggaagactgttgcgacgccgtatgctaagaagcttgctaaacaacacaaggttgatattgaatccgttgctggaactggaccattcggtaggattacggcttctgatgtggagacggcggctggaattgctccgtccaaatcctccatcgcaccaccgcctcctcctccacctccggtgacggctaaagcaaccaccactaatttgcctcctctgttacctgattcaagcattgttcctttcacagcaatgcaatctgcagtatctaagaacatgattgagagtctctctgttcctacattccgtgttggttatcctgtgaacactgacgctcttgatgcactttacgagaaggtgaagccaaagggtgtaacaatgacagctttattagctaaagctgcagggatggccttggctcagcatcctgtggtgaacgctagctgcaaagacgggaagagttttagttacaatagtagcattaacattgcagtggcggttgctatcaatggtggcctgattacgcctgttctacaagatgcagataagttggatttgtacttgttatctcaaaaatggaaagagctggtggggaaagctagaagcaagcaacttcaaccccatgaatacaactctggaacttttactttatcgaatctcggtatgtttggagtggatagatttgacgctattcttccgccaggacagggtgctattatggctgttggagcgtcaaagccaactgtagttgctgataaggatggattcttcagtgtaaaaaacacaatgctggtgaatgtgactgcagatcatcgcattgtgtatggagctgacttggctgcttttctccaaacctttgcaaagatcattgagaatccagatagtttgaccttataagacgccaagcgaagacgagaagtcaaaaacagtttccaaaattcctgagccaaatttttcccaagtaaattttttaatcttcattgttcttggtcttgctctacttcttttgcatctttttcttcacttgtgttgtatctgtatttttgttttcaagaatcatcattttgggttttaaacaaataatttcctatccagaatc

SEQ ID NO: 59 - DNA, 46 bp, Artificial Sequence
  Synthetic oligonucleotide
atactaggatccgtttaaacctgcagatggagaaaaaaatcactgg

SEQ ID NO: 60 - DNA, 31 bp, Artificial Sequence
  Synthetic oligonucleotide
cacgtgggtaccctcgagaagcttttacgcc

SEQ ID NO: 61 - DNA, 41 bp, Artificial Sequence
  Synthetic oligonucleotide
ctttatagaccatggaggcaaaccttatggccgaggagatg

SEQ ID NO: 62 - DNA, 37 bp, Artificial Sequence
  Synthetic oligonucleotide
ccttgagaagcttgcatgctcaggccgtcaccggcgg

SEQ ID NO: 63 - DNA, 554 bp, Rhodobacter capsulatus
catggaggcaaaccttatggccgaggagatgatccccgcctgggtcgagggcgtgctgcaacccgtcgagaagctggaggcccaccgcaagggcctgcggcatctggcgatttcggtcttcgtgacgcgcggcaacaaggtgcttttgcagcaacgcgcgctgtcgaaatatcacacgccggggctttgggcgaatacctgctgcacccatccctattggggcgaggatgcgccgacctgcgccgcccgccgtctggggcaggagctgggcatcgtcgggctgaagctgcgccacatggggcagctggaataccgcgccgatgtgaacaacggcatgatcgagcatgaggtggtggaggtcttcaccgccgaagcgcccgaggggatcgagccgcaacccgaccccgaggaagtggccgataccgaatgggtgcgcatcgacgcgctgcgctcggagatccacgccaatccggaacgcttcacgccctggctcaagatctatatcgagcagcaccgcgacatgatctttccgccggtgacggcctgagcatgca

SEQ ID NO: 64 - DNA, 34 bp, Artificial Sequence
  Synthetic oligonucleotide
tacctcatgacctagcagcaccaccacaatatgc

SEQ ID NO: 65 - DNA, 118 bp, Synechocystis sp PCC6803
  Synthetic oligonucleotide
cctagcagcaccaccacaatatgcccccaccttaatcctgggttatttttaagttattgctccactccctccagttgatggcaaaattgcttgccggtatttgtaatgtaattcactg

SEQ ID NO: 66 - DNA, 167 bp, Synechocystis sp PCC6803
gggacattttgctctggttgacgatacagtgaagcttggactggttgaccccgatagctgcggagtagggcatcaagccacagttttcctttaataatccccccatgaaatggcataaagagagcaaagtattactacaaggagtacatcatcccctcggtttaacc

SEQ ID NO: 67 - DNA, 1345 bp, Synechocystis sp PCC6803
catgacctagcagcaccaccacaatatgcccccaccttaatcctgggttatttttaagttattgctccactccctccagttgatggcaaaattgcttgccggtatttgtaatgtaattcactgatggatagcaccccccaccgtaagtccgatcatatccgcattgtcctagaagaagatgtggtgggcaaaggcatttccaccggctttgaaagattgatgctggaacactgcgctcttcctgcggtggatctggatgcagtggatttgggactgaccctctggggtaaatccttgacttacccttggttgatcagcagtatgaccggcggcacgccagaggccaagcaaattaatctatttttagccgaggtggcccaggctttgggcatcgccatgggtttgggttcccaacgggccgccattgaaaatcctgatttagccttcacctatcaagtccgctccgtcgccccagatattttactttttgccaacctgggattagtgcaattaaattacggttacggtttggagcaagcccagcgggcggtggatatgattgaagccgatgcgctgattttgcatctcaatcccctccaggaagcggtgcaacccgatggcgatcgcctgtggtcgggactctggtctaagttagaagctttagtagaggctttggaagtgccggtaattgtcaaagaagtgggcaatggcattagcggtccggtggccaaaagattgcaggaatgtggggtcggggcgatcgatgtggctggagctgggggcaccagttggagtgaagtggaagcccatcgacaaaccgatcgccaagcgaaggaagtggcccataactttgccgattggggattacccacagcctggagtttgcaacaggtagtgcaaaatactgagcagatcctggttttcgccagcggcggcattcgttccggcattgacggggccaaggcgatcgccctgggggccaccctggtgggtagtgcggcaccggtattagcagaagcgaaaatcaacgcccaaagggtttatgaccattaccaggcacggctaagggaactgcaaatcgccgccttttgttgtgatgccgccaatctgacccaactggcccaagtccccctttgggacagacaatcgggacaaaggttaactaaaccttaagggacattttgctctggttgacgatacagtgaagcttggactggttgaccccgatagctgcggagtagggcatcaagccacagttttcctttaataatccccccatgaaatggcataaagagagcaaagtattactacaaggagtacatcatcccctcggtttaaccgcatg

SEQ ID NO: 68 - DNA, 33 bp, Artificial Sequence
  Synthetic oligonucleotide
ctataccgaattccgaaaccttgctctcactag

SEQ ID NO: 69 - DNA, 34 bp, Artificial Sequence
  Synthetic oligonucleotide
ccgtatatctagagggcgattaatttacccaaac

SEQ ID NO: 70 - DNA, 4105 bp, Synechocystis sp. PCC 6803
ctataccgaattccgaaaccttgctctcactaggaatgcccctgggcaacggattaccagccgcaacagtggcccaagcctatgttcatagcttagaaggcactatgacaggagaagtgctctatccgtagtaaccatatcttggtttactcttcccccatcatggattggagataattttccagtccagaattactgataagccattgctgggactctaaccagtcaatttgttcttctgtttcttcaagaatttccgacaacacatcccggcttacatagtcccgttgggtttcaaagaaggcaatgctgttaactaaaccatccctaatgccttggttcatggtcagatcattgcccaggatttccggtaccgtctcgccgatgagaagtttttccaaattttggagattggggagtccttccaaaaataaaacccgctcgatcaggctatcggcctgcttcattgccttgatggatactttatattcgtactgattaagtgcgttcagcccccaatttttgcacatgcgagcatggagaaaatattggttaatcgcagtaagttgtagctttaacgcttggttgagatgttgtctgacttccaggttgccttccatgttgttatcctctgatgtggagttttgtttgatgttgttgtttccatttttacccattcacggtccgacgacggagttatttactgggacagcaataaattgtttaaattgttttaatgttttacccctgggaaaattgcctttttctcaaaggaagtgtccctctctgaccttaaactgaaccaatatggctgatttgtttgtcggtgccccagttcgtttaattgcccgtcccccctatttgaaaaccgctgatcccatgcccatgctccgtcctccggatttattggcgatcgccgcggagggaatggtggtagaccgtcgaccggctggctattggggagtaaagtttgaccgaggcacttttctgttggaaagccagtatttggaagtgattcggcctcaggaagaaaaaacggaagtctcggattaagaacgccgagtaaatgaccaagtttaatctaaaaatatggcatcaactgtaaatcgcctttttttagcaattttgaccatagccagcttcagccttagtggaggttatggatatgttcccgttcccatggcgatcgccgctgacgtcccagaactgacagcaaaggtgcccaattatttggataaaatccaatttcctctaggggttatcgatgtctatggattgatgggcccagaggatggtaaacgttcccaaggctatgaattttgtgttgtgcccgagaaaaaaagtgaagttttggccatcgatccctcactcacattttcgtctagccctggtcgcatcggttgcccccaggaacaattactgtgcctaggagatacccagcaaccaaattggcaggccattctctttgccctggcccggttgagttacatagaaaaaatcttgccccactggggagaatagaagcccctatttgacaaatgtttctggccaagggacaggggaagcatctagtgcaagggatacctttccgttaagatggttaacgctgaacaattgagcgcattgctaaccaggcggccctgcgacagccccaagctgtcccccgttttgctggcgatcggccgttgacccagcacgaaaactcttcttttatagttaaaggtattgtaatgaatcaggaaatttttgaaaaagtaaaaaaaatcgtcgtggaacagttggaagtggatcctgacaaagtgacccccgatgccacctttgccgaagatttaggggctgattccctcgatacagtggaattggtcatggccctggaagaagagtttgatattgaaattcccgatgaagtggcggaaaccattgataccgtgggcaaagccgttgagcatatcgaaagtaaataaattccggccatagccccgactccccccatagatctttggagccgagttctcggacggtttaagccactgtttaggactgccccaatgccggttttgggtttatcagtttgcccctcgggctaggccctggccccgtcgctgtatctttgcggagaactccaggggagtcccctccccgattctatctattaagtaccatggcaaatttggaaaagaaacgtgttgttgtaacgggattgggagccatcacccccatcggtaatactctccaagactattggcaaggcttaatggagggtcgtaacggcattggccccattacccgtttcgatgctagtgaccaagcctgccgttttggaggggaagtaaaggattttgatgctacccagtttcttgaccgcaaagaagctaaacggatggaccggttttgccattttgctgtttgtgccagtcaacaggcaattaacgatgctaagttggtgattaacgaactcaatgccgatgaaatcggggtattgattggcacgggcattggtggtttgaaagtactggaagatcaacaaaccattctgttggataagggtcctagccgttgcagtccttttatgatcccgatgatgatcgccaacatggcctctgggttaaccgccatcaacttaggggccaagggtcccaataactgtacggtgacggcctgtgcggcgggttccaatgccattggagatgcgtttcgtttggtgcaaaatggctatgctaaggcaatgatttgcggtggcacggaagcggccattaccccgctgagctatgcaggttttgcttcggcccgggctttatctttccgcaatgatgatcccctccatgccagtcgtcccttcgataaggaccgggatggttttgtgatgggggaaggatcgggcattttgatcctagaagaattggaatccgccttggcccggggagcaaaaatttatggggaaatggtgggctatgccatgacctgtgatgcctatcacattaccgccccagtgccggatggtcggggagccaccagggcgatcgcctgggccttaaaagacagcggattgaaaccggaaatggtcagttacatcaatgcccatggtaccagcacccctgctaacgatgtgacggaaacccgtgccattaaacaggcgttgggaaatcatgcctacaatattgcggttagttctactaagtctatgaccggtcacttgttgggcggctccggaggtatcgaagcggtggccaccgtaatggcgatcgccgaagataaggtaccccccaccattaatttggagaaccccgaccctgagtgtgatttggattatgtgccggggcagagtcgggctttaatagtggatgtagccctatccaactcctttggttttggtggccataacgtcaccttagctttcaaaaaatatcaatagcccaccgaaaaatttcccgaaccgtgggaagatggtagcaatttggcctgccttggcccctaccattaccgccccccggtggatattgacccaattattgctagtttatttttccaaacattatggtcgttgctacccagtccttagacgaactttctattaatgccattcgctttttagccgttgacgccattgaaaaggccaaatctggccaccctggtttgcccatgggagccgctcctatggcctttaccctgtggaacaagttcatgaagttcaatcccaagaaccccaagtggttcaatcgggaccgctttgtgttgtccgccggccatggctccatgttgcagtatgccctgctctatctgctgggttatgacagtgtgaccatcgaagacattaaacagttccgtcaatgggaatcttctacccccggtcacccggagaattttctcactgctggagtagaagtcaccaccggccccttgggtcaaggcattgccaatggtgtgggtttagccctggcggaagcccatttggctgccacctacaacaagcctgatgccaccattgtggaccattacacctatgtgattctgggggatggttgcaatatggaaggtatttccggggaagccgcttccattgcagggcattggggtttgggtaaattaatcgccctctagatatacg

SEQ ID NO: 71 - DNA, 2686 bp, Artificial Sequence
  Vector sequence
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 72 - DNA, 2665 bp, Artificial Sequence
  Vector sequence
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 73 - DNA, 6745 bp, Artificial Sequence
  Vector sequence
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccgaaaccttgctctcactaggaatgcccctgggcaacggattaccagccgcaacagtggcccaagcctatgttcatagcttagaaggcactatgacaggagaagtgctctatccgtagtaaccatatcttggtttactcttcccccatcatggattggagataattttccagtccagaattactgataagccattgctgggactctaaccagtcaatttgttcttctgtttcttcaagaatttccgacaacacatcccggcttacatagtcccgttgggtttcaaagaaggcaatgctgttaactaaaccatccctaatgccttggttcatggtcagatcattgcccaggatttccggtaccgtctcgccgatgagaagtttttccaaattttggagattggggagtccttccaaaaataaaacccgctcgatcaggctatcggcctgcttcattgccttgatggatactttatattcgtactgattaagtgcgttcagcccccaatttttgcacatgcgagcatggagaaaatattggttaatcgcagtaagttgtagctttaacgcttggttgagatgttgtctgacttccaggttgccttccatgttgttatcctctgatgtggagttttgtttgatgttgttgtttccatttttacccattcacggtccgacgacggagttatttactgggacagcaataaattgtttaaattgttttaatgttttacccctgggaaaattgcctttttctcaaaggaagtgtccctctctgaccttaaactgaaccaatatggctgatttgtttgtcggtgccccagttcgtttaattgcccgtcccccctatttgaaaaccgctgatcccatgcccatgctccgtcctccggatttattggcgatcgccgcggagggaatggtggtagaccgtcgaccggctggctattggggagtaaagtttgaccgaggcacttttctgttggaaagccagtatttggaagtgattcggcctcaggaagaaaaaacggaagtctcggattaagaacgccgagtaaatgaccaagtttaatctaaaaatatggcatcaactgtaaatcgcctttttttagcaattttgaccatagccagcttcagccttagtggaggttatggatatgttcccgttcccatggcgatcgccgctgacgtcccagaactgacagcaaaggtgcccaattatttggataaaatccaatttcctctaggggttatcgatgtctatggattgatgggcccagaggatggtaaacgttcccaaggctatgaattttgtgttgtgcccgagaaaaaaagtgaagttttggccatcgatccctcactcacattttcgtctagccctggtcgcatcggttgcccccaggaacaattactgtgcctaggagatacccagcaaccaaattggcaggccattctctttgccctggcccggttgagttacatagaaaaaatcttgccccactggggagaatagaagcccctatttgacaaatgtttctggccaagggacaggggaagcatctagtgcaagggatacctttccgttaagatggttaacgctgaacaattgagcgcattgctaaccaggcggccctgcgacagccccaagctgtcccccgttttgctggcgatcggccgttgacccagcacgaaaactcttcttttatagttaaaggtattgtaatgaatcaggaaatttttgaaaaagtaaaaaaaatcgtcgtggaacagttggaagtggatcctgacaaagtgacccccgatgccacctttgccgaagatttaggggctgattccctcgatacagtggaattggtcatggccctggaagaagagtttgatattgaaattcccgatgaagtggcggaaaccattgataccgtgggcaaagccgttgagcatatcgaaagtaaataaattccggccatagccccgactccccccatagatctttggagccgagttctcggacggtttaagccactgtttaggactgccccaatgccggttttgggtttatcagtttgcccctcgggctaggccctggccccgtcgctgtatctttgcggagaactccaggggagtcccctccccgattctatctattaagtaccatggcaaatttggaaaagaaacgtgttgttgtaacgggattgggagccatcacccccatcggtaatactctccaagactattggcaaggcttaatggagggtcgtaacggcattggccccattacccgtttcgatgctagtgaccaagcctgccgttttggaggggaagtaaaggattttgatgctacccagtttcttgaccgcaaagaagctaaacggatggaccggttttgccattttgctgtttgtgccagtcaacaggcaattaacgatgctaagttggtgattaacgaactcaatgccgatgaaatcggggtattgattggcacgggcattggtggtttgaaagtactggaagatcaacaaaccattctgttggataagggtcctagccgttgcagtccttttatgatcccgatgatgatcgccaacatggcctctgggttaaccgccatcaacttaggggccaagggtcccaataactgtacggtgacggcctgtgcggcgggttccaatgccattggagatgcgtttcgtttggtgcaaaatggctatgctaaggcaatgatttgcggtggcacggaagcggccattaccccgctgagctatgcaggttttgcttcggcccgggctttatctttccgcaatgatgatcccctccatgccagtcgtcccttcgataaggaccgggatggttttgtgatgggggaaggatcgggcattttgatcctagaagaattggaatccgccttggcccggggagcaaaaatttatggggaaatggtgggctatgccatgacctgtgatgcctatcacattaccgccccagtgccggatggtcggggagccaccagggcgatcgcctgggccttaaaagacagcggattgaaaccggaaatggtcagttacatcaatgcccatggtaccagcacccctgctaacgatgtgacggaaacccgtgccattaaacaggcgttgggaaatcatgcctacaatattgcggttagttctactaagtctatgaccggtcacttgttgggcggctccggaggtatcgaagcggtggccaccgtaatggcgatcgccgaagataaggtaccccccaccattaatttggagaaccccgaccctgagtgtgatttggattatgtgccggggcagagtcgggctttaatagtggatgtagccctatccaactcctttggttttggtggccataacgtcaccttagctttcaaaaaatatcaatagcccaccgaaaaatttcccgaaccgtgggaagatggtagcaatttggcctgccttggcccctaccattaccgccccccggtggatattgacccaattattgctagtttatttttccaaacattatggtcgttgctacccagtccttagacgaactttctattaatgccattcgctttttagccgttgacgccattgaaaaggccaaatctggccaccctggtttgcccatgggagccgctcctatggcctttaccctgtggaacaagttcatgaagttcaatcccaagaaccccaagtggttcaatcgggaccgctttgtgttgtccgccggccatggctccatgttgcagtatgccctgctctatctgctgggttatgacagtgtgaccatcgaagacattaaacagttccgtcaatgggaatcttctacccccggtcacccggagaattttctcactgctggagtagaagtcaccaccggccccttgggtcaaggcattgccaatggtgtgggtttagccctggcggaagcccatttggctgccacctacaacaagcctgatgccaccattgtggaccattacacctatgtgattctgggggatggttgcaatatggaaggtatttccggggaagccgcttccattgcagggcattggggtttgggtaaattaatcgccctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 74 - DNA, 44 bp, Artificial Sequence
  Multiple cloning site
agatcttgatcagatatcacgcgtgtttaaacactagtggatcc

SEQ ID NO: 75 - DNA, 6783 bp, Artificial Sequence
  Vector sequence
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccgaaaccttgctctcactaggaatgcccctgggcaacggattaccagccgcaacagtggcccaagcctatgttcatagcttagaaggcactatgacaggagaagtgctctatccgtagtaaccatatcttggtttactcttcccccatcatggattggagataattttccagtccagaattactgataagccattgctgggactctaaccagtcaatttgttcttctgtttcttcaagaatttccgacaacacatcccggcttacatagtcccgttgggtttcaaagaaggcaatgctgttaactaaaccatccctaatgccttggttcatggtcagatcattgcccaggatttccggtaccgtctcgccgatgagaagtttttccaaattttggagattggggagtccttccaaaaataaaacccgctcgatcaggctatcggcctgcttcattgccttgatggatactttatattcgtactgattaagtgcgttcagcccccaatttttgcacatgcgagcatggagaaaatattggttaatcgcagtaagttgtagctttaacgcttggttgagatgttgtctgacttccaggttgccttccatgttgttatcctctgatgtggagttttgtttgatgttgttgtttccatttttacccattcacggtccgacgacggagttatttactgggacagcaataaattgtttaaattgttttaatgttttacccctgggaaaattgcctttttctcaaaggaagtgtccctctctgaccttaaactgaaccaatatggctgatttgtttgtcggtgccccagttcgtttaattgcccgtcccccctatttgaaaaccgctgatcccatgcccatgctccgtcctccggatttattggcgatcgccgcggagggaatggtggtagaccgtcgaccggctggctattggggagtaaagtttgaccgaggcacttttctgttggaaagccagtatttggaagtgattcggcctcaggaagaaaaaacggaagtctcggattaagaacgccgagtaaatgaccaagtttaatctaaaaatatggcatcaactgtaaatcgcctttttttagcaattttgaccatagccagcttcagccttagtggaggttatggatatgttcccgttcccatggcgatcgccgctgacgtcccagaactgacagcaaaggtgcccaattatttggataaaatccaatttcctctaggggttatcgatgtctatggattgatgggcccagaggatggtaaacgttcccaaggctatgaattttgtgttgtgcccgagaaaaaaagtgaagttttggccatcgatccctcactcacattttcgtctagccctggtcgcatcggttgcccccaggaacaattactgtgcctaggagatacccagcaaccaaattggcaggccattctctttgccctggcccggttgagttacatagaaaaaatcttgccccactggggagaatagaagcccctatttgacaaatgtttctggccaagggacaggggaagcatctagtgcaagggatacctttccgttaagatggttaacgctgaacaattgagcgcattgctaaccaggcggccctgcgacagccccaagctgtcccccgttttgctggcgatcggccgttgacccagcacgaaaactcttcttttatagttaaaggtattgtaatgaatcaggaaatttttgaaaaagtaaaaaaaatcgtcgtggaacagttggaagtggatcctgacaaagtgacccccgatgccacctttgccgaagatttaggggctgattccctcgatacagtggaattggtcatggccctggaagaagagtttgatattgaaattcccgatgaagtggcggaaaccattgataccgtgggcaaagccgttgagcatatcgaaagtaaataaattccggccatagccccgactccccccatagatcttgatcagatatcacgcgtgtttaaacactagtggatctttggagccgagttctcggacggtttaagccactgtttaggactgccccaatgccggttttgggtttatcagtttgcccctcgggctaggccctggccccgtcgctgtatctttgcggagaactccaggggagtcccctccccgattctatctattaagtaccatggcaaatttggaaaagaaacgtgttgttgtaacgggattgggagccatcacccccatcggtaatactctccaagactattggcaaggcttaatggagggtcgtaacggcattggccccattacccgtttcgatgctagtgaccaagcctgccgttttggaggggaagtaaaggattttgatgctacccagtttcttgaccgcaaagaagctaaacggatggaccggttttgccattttgctgtttgtgccagtcaacaggcaattaacgatgctaagttggtgattaacgaactcaatgccgatgaaatcggggtattgattggcacgggcattggtggtttgaaagtactggaagatcaacaaaccattctgttggataagggtcctagccgttgcagtccttttatgatcccgatgatgatcgccaacatggcctctgggttaaccgccatcaacttaggggccaagggtcccaataactgtacggtgacggcctgtgcggcgggttccaatgccattggagatgcgtttcgtttggtgcaaaatggctatgctaaggcaatgatttgcggtggcacggaagcggccattaccccgctgagctatgcaggttttgcttcggcccgggctttatctttccgcaatgatgatcccctccatgccagtcgtcccttcgataaggaccgggatggttttgtgatgggggaaggatcgggcattttgatcctagaagaattggaatccgccttggcccggggagcaaaaatttatggggaaatggtgggctatgccatgacctgtgatgcctatcacattaccgccccagtgccggatggtcggggagccaccagggcgatcgcctgggccttaaaagacagcggattgaaaccggaaatggtcagttacatcaatgcccatggtaccagcacccctgctaacgatgtgacggaaacccgtgccattaaacaggcgttgggaaatcatgcctacaatattgcggttagttctactaagtctatgaccggtcacttgttgggcggctccggaggtatcgaagcggtggccaccgtaatggcgatcgccgaagataaggtaccccccaccattaatttggagaaccccgaccctgagtgtgatttggattatgtgccggggcagagtcgggctttaatagtggatgtagccctatccaactcctttggttttggtggccataacgtcaccttagctttcaaaaaatatcaatagcccaccgaaaaatttcccgaaccgtgggaagatggtagcaatttggcctgccttggcccctaccattaccgccccccggtggatattgacccaattattgctagtttatttttccaaacattatggtcgttgctacccagtccttagacgaactttctattaatgccattcgctttttagccgttgacgccattgaaaaggccaaatctggccaccctggtttgcccatgggagccgctcctatggcctttaccctgtggaacaagttcatgaagttcaatcccaagaaccccaagtggttcaatcgggaccgctttgtgttgtccgccggccatggctccatgttgcagtatgccctgctctatctgctgggttatgacagtgtgaccatcgaagacattaaacagttccgtcaatgggaatcttctacccccggtcacccggagaattttctcactgctggagtagaagtcaccaccggccccttgggtcaaggcattgccaatggtgtgggtttagccctggcggaagcccatttggctgccacctacaacaagcctgatgccaccattgtggaccattacacctatgtgattctgggggatggttgcaatatggaaggtatttccggggaagccgcttccattgcagggcattggggtttgggtaaattaatcgccctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 76 - DNA, 816 bp, Salmonella enterica
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 77 - DNA, 39 bp, Artificial Sequence
  Synthetic oligonucleotide
ctatacctgatcataaacagtaatacaaggggtgttatg

SEQ ID NO: 78 - DNA, 35 bp, Artificial Sequence
  Synthetic oligonucleotide
ccgtataacgcgtttagaaaaactcatcgagcatc

SEQ ID NO: 79 - DNA, 865 bp, Salmonella enterica
ctatacctgatcataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaacgcgttatacgg

SEQ ID NO: 80 - DNA, 7616 bp, Artificial Sequence
  Vector sequence
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccgaaaccttgctctcactaggaatgcccctgggcaacggattaccagccgcaacagtggcccaagcctatgttcatagcttagaaggcactatgacaggagaagtgctctatccgtagtaaccatatcttggtttactcttcccccatcatggattggagataattttccagtccagaattactgataagccattgctgggactctaaccagtcaatttgttcttctgtttcttcaagaatttccgacaacacatcccggcttacatagtcccgttgggtttcaaagaaggcaatgctgttaactaaaccatccctaatgccttggttcatggtcagatcattgcccaggatttccggtaccgtctcgccgatgagaagtttttccaaattttggagattggggagtccttccaaaaataaaacccgctcgatcaggctatcggcctgcttcattgccttgatggatactttatattcgtactgattaagtgcgttcagcccccaatttttgcacatgcgagcatggagaaaatattggttaatcgcagtaagttgtagctttaacgcttggttgagatgttgtctgacttccaggttgccttccatgttgttatcctctgatgtggagttttgtttgatgttgttgtttccatttttacccattcacggtccgacgacggagttatttactgggacagcaataaattgtttaaattgttttaatgttttacccctgggaaaattgcctttttctcaaaggaagtgtccctctctgaccttaaactgaaccaatatggctgatttgtttgtcggtgccccagttcgtttaattgcccgtcccccctatttgaaaaccgctgatcccatgcccatgctccgtcctccggatttattggcgatcgccgcggagggaatggtggtagaccgtcgaccggctggctattggggagtaaagtttgaccgaggcacttttctgttggaaagccagtatttggaagtgattcggcctcaggaagaaaaaacggaagtctcggattaagaacgccgagtaaatgaccaagtttaatctaaaaatatggcatcaactgtaaatcgcctttttttagcaattttgaccatagccagcttcagccttagtggaggttatggatatgttcccgttcccatggcgatcgccgctgacgtcccagaactgacagcaaaggtgcccaattatttggataaaatccaatttcctctaggggttatcgatgtctatggattgatgggcccagaggatggtaaacgttcccaaggctatgaattttgtgttgtgcccgagaaaaaaagtgaagttttggccatcgatccctcactcacattttcgtctagccctggtcgcatcggttgcccccaggaacaattactgtgcctaggagatacccagcaaccaaattggcaggccattctctttgccctggcccggttgagttacatagaaaaaatcttgccccactggggagaatagaagcccctatttgacaaatgtttctggccaagggacaggggaagcatctagtgcaagggatacctttccgttaagatggttaacgctgaacaattgagcgcattgctaaccaggcggccctgcgacagccccaagctgtcccccgttttgctggcgatcggccgttgacccagcacgaaaactcttcttttatagttaaaggtattgtaatgaatcaggaaatttttgaaaaagtaaaaaaaatcgtcgtggaacagttggaagtggatcctgacaaagtgacccccgatgccacctttgccgaagatttaggggctgattccctcgatacagtggaattggtcatggccctggaagaagagtttgatattgaaattcccgatgaagtggcggaaaccattgataccgtgggcaaagccgttgagcatatcgaaagtaaataaattccggccatagccccgactccccccatagatcttgatcataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaacgcgtgtttaaacactagtggatctttggagccgagttctcggacggtttaagccactgtttaggactgccccaatgccggttttgggtttatcagtttgcccctcgggctaggccctggccccgtcgctgtatctttgcggagaactccaggggagtcccctccccgattctatctattaagtaccatggcaaatttggaaaagaaacgtgttgttgtaacgggattgggagccatcacccccatcggtaatactctccaagactattggcaaggcttaatggagggtcgtaacggcattggccccattacccgtttcgatgctagtgaccaagcctgccgttttggaggggaagtaaaggattttgatgctacccagtttcttgaccgcaaagaagctaaacggatggaccggttttgccattttgctgtttgtgccagtcaacaggcaattaacgatgctaagttggtgattaacgaactcaatgccgatgaaatcggggtattgattggcacgggcattggtggtttgaaagtactggaagatcaacaaaccattctgttggataagggtcctagccgttgcagtccttttatgatcccgatgatgatcgccaacatggcctctgggttaaccgccatcaacttaggggccaagggtcccaataactgtacggtgacggcctgtgcggcgggttccaatgccattggagatgcgtttcgtttggtgcaaaatggctatgctaaggcaatgatttgcggtggcacggaagcggccattaccccgctgagctatgcaggttttgcttcggcccgggctttatctttccgcaatgatgatcccctccatgccagtcgtcccttcgataaggaccgggatggttttgtgatgggggaaggatcgggcattttgatcctagaagaattggaatccgccttggcccggggagcaaaaatttatggggaaatggtgggctatgccatgacctgtgatgcctatcacattaccgccccagtgccggatggtcggggagccaccagggcgatcgcctgggccttaaaagacagcggattgaaaccggaaatggtcagttacatcaatgcccatggtaccagcacccctgctaacgatgtgacggaaacccgtgccattaaacaggcgttgggaaatcatgcctacaatattgcggttagttctactaagtctatgaccggtcacttgttgggcggctccggaggtatcgaagcggtggccaccgtaatggcgatcgccgaagataaggtaccccccaccattaatttggagaaccccgaccctgagtgtgatttggattatgtgccggggcagagtcgggctttaatagtggatgtagccctatccaactcctttggttttggtggccataacgtcaccttagctttcaaaaaatatcaatagcccaccgaaaaatttcccgaaccgtgggaagatggtagcaatttggcctgccttggcccctaccattaccgccccccggtggatattgacccaattattgctagtttatttttccaaacattatggtcgttgctacccagtccttagacgaactttctattaatgccattcgctttttagccgttgacgccattgaaaaggccaaatctggccaccctggtttgcccatgggagccgctcctatggcctttaccctgtggaacaagttcatgaagttcaatcccaagaaccccaagtggttcaatcgggaccgctttgtgttgtccgccggccatggctccatgttgcagtatgccctgctctatctgctgggttatgacagtgtgaccatcgaagacattaaacagttccgtcaatgggaatcttctacccccggtcacccggagaattttctcactgctggagtagaagtcaccaccggccccttgggtcaaggcattgccaatggtgtgggtttagccctggcggaagcccatttggctgccacctacaacaagcctgatgccaccattgtggaccattacacctatgtgattctgggggatggttgcaatatggaaggtatttccggggaagccgcttccattgcagggcattggggtttgggtaaattaatcgccctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 81 - DNA, 38 bp, Artificial Sequence
  Synthetic oligonucleotide
ctttatagagtcgactgtgattcaacaatggcggtttc

SEQ ID NO: 82 - DNA, 35 bp, Artificial Sequence
  Synthetic oligonucleotide
gaaagtcgacttataaggtcaaactatctggattc

SEQ ID NO: 83 - DNA, 37 bp, Artificial Sequence
  Synthetic oligonucleotide
caggtttgcggccgcaagaaattcaaaaacgagtagc

SEQ ID NO: 84 - DNA, 42 bp, Artificial Sequence
  Synthetic oligonucleotide
aagacccgggatcctaggtcgtatattttcttccgtatttat

SEQ ID NO: 85 - DNA, 1251 bp, Synechocystis sp. PCC 6803
ctattgatattttttgaaagctaaggtgacgttatggccaccaaaaccaaaggagttggatagggctacatccactattaaagcccgactctgccccggcacataatccaaatcacactcagggtcggggttctccaaattaatggtggggggtaccttatcttcggcgatcgccattacggtggccaccgcttcgatacctccggagccgcccaacaagtgaccggtcatagacttagtagaactaaccgcaatattgtaggcatgatttcccaacgcctgtttaatggcacgggtttccgtcacatcgttagcaggggtgctggtaccatgggcattgatgtaactgaccatttccggtttcaatccgctgtcttttaaggcccaggcgatcgccctggtggctccccgaccatccggcactggggcggtaatgtgataggcatcacaggtcatggcatagcccaccatttccccataaatttttgctccccgggccaaggcggattccaattcttctaggatcaaaatgcccgatccttcccccatcacaaaaccatcccggtccttatcgaagggacgactggcatggaggggatcatcattgcggaaagataaagcccgggccgaagcaaaacctgcatagctcagcggggtaatggccgcttccgtgccaccgcaaatcattgccttagcatagccattttgcaccaaacgaaacgcatctccaatggcattggaacccgccgcacaggccgtcaccgtacagttattgggacccttggcccctaagttgatggcggttaacccagaggccatgttggcgatcatcatcgggatcataaaaggactgcaacggctaggacccttatccaacagaatggtttgttgatcttccagtactttcaaaccaccaatgcccgtgccaatcaataccccgatttcatcggcattgagttcgttaatcaccaacttagcatcgttaattgcctgttgactggcacaaacagcaaaatggcaaaaccggtccatccgtttagcttctttgcggtcaagaaactgggtagcatcaaaatcctttacttcccctccaaaacggcaggcttggtcactagcatcgaaacgggtaatggggccaatgccgttacgaccctccattaagccttgccaatagtcttggagagtattaccgatgggggtgatggctcccaatcccgttacaacaacacgtttcttttccaaatttgccat

SEQ ID NO: 86 - DNA, 1638 bp, Phaeodactylum tricornutum
atggctccgcaacaacgaaaccccgtactcaatgaagacggaaacacggggatgcgacgggtggactccgaggcttccgacatgagtgaactcggcaacgatacacgagcgcaagactatcgcatccgtaagagttccttgattggaatgatcgactgggggcacgttatggtgtcccatcttcccttgctaatggtcgtgggtatcctgacgctggtggcgcagattgtgcaccaggttgttattgaactcggtctgcaaaacattgactggtccgtgcagaccgtgtcgaccatctgtcacgccatcaaggagctctttcgcgatttgtacgcttccattatggaaagccgcggctttgacttattctcccccgccgtcaaaaccaccgccctcctgttgttcctcggcgcctggtggatgagacgcaagagtcccgtctatcttttgtcctttgcaaccttcaaggccccggattcttggaaaatgtcgcacgcacagattgtggaaattatgcgccgtcaagggtgcttttccgaagactcgctcgaattcatgggcaaaattctggcgcgctcgggtaccggccaagccacggcttggcctccgggcataacccgctgtctacaggacgaaaacaccaaagccgatcggtccatcgaagcggcacgccgcgaagccgaaatcgtcatctttgacgtcgtcgaaaaggctctccaaaaagcccgcgtccggccccaagacattgacattctcattatcaactgcagtttgttcagcccaactccctcgttgtgcgccatggtactgtcccactttggcatgcgcagcgacgttgccaccttcaatttgtccggcatgggctgttccgcctcgctcattagcatcgatctcgccaaatccctcttgggcacccggccgaatagcaaggccctcgtggtgagtacggaaatcatcacgcccgccttgtaccacggcagcgaccggggctttttgatccaaaacacactcttccgctgtggcggagccgctatggtgttgagcaattcctggtacgacggtcgccgcgcctggtacaagctgctacacacggtccgggtgcagggcaccaacgaagccgccgtctcgtgcgtctacgaaaccgaagacgcccagggacatcagggtgtacgcttgagtaaggatatcgtcaaggtggcgggcaaatgcatggaaaagaactttaccgttttgggtccgtccgtgctgccgctgacggagcaagccaaggtggtggtgtcgattgccgcccggtttgttctgaaaaagttcgaagggtacacgaaacgcaaggtaccgtcgattcggccgtacgtgccggatttcaaacgcggcatcgaccacttttgtatccacgccgggggacgtgccgtgattgacggtatcgaaaagaatatgcagctgcaaatgtaccacaccgaggcgtcgcgtatgacgctactgaattacggcaacacgagcagcagcagtatctggtacgagttggagtacattcaggaccagcaaaagacgaatccgctgaaaaagggcgaccgggtattgcaagtggcgttcgggtccggcttcaagtgcacgtccggggtgtggctcaagctctaa

SEQ ID NO: 87 - DNA, 1246 bp, Arabidopsis thaliana
atggctaatgcatctgggttcttcactcatccttcaattcctaacttgcgaagcagaatccatgttccggttagagtttctggatctgggttttgcgtttccaatcgattctctaagagggttttgtgctctagcgtcagctccgtcgataaggatgcttcgtcttctccttctcaatatcaacgacccaggctagtgccgagtggctgcaaattgattggatgtggatcagcagttccaagtcttctgatttctaatgatgatctcgctaaaatagttgatactaatgatgaatggattgctactcgtactggtattcgcaaccgtcgagttgtctcaggcaaagatagcttggttggcttagcagtagaagcagcaaccaaagctcttgaaatggctgaggttgttcctgaagatattgacttagtcttgatgtgtacttccactcctgatgatctatttggtgctgctccacagattcaaaaggcacttggttgcacaaagaacccattggcttatgatatcacagctgcttgtagtggatttgttttgggtctagtttcagctgcttgtcatataaggggaggcggttttaagaacgttttagtgatcggagctgattctttgtctcggtttgttgattggacggatagagggacttgcattctatttggagatgctgctggtgctgtggttgttcaggcttgtgatattgaagatgatggtttgttcagttttgatgtgcacagcgatggggatggtcgaagacatttgaatgcttctgttaaagaatcccaaaacgatggtgaatcaagctccaatggctcggtgtttggagactttccaccaaaacaatcttcatattcttgtattcagatgaatggaaaagaggtctttcgctttgctgtcaaatgtgttcctcaatctattgaatctgctttacaaaaagctggtcttcctgcttctgccatcgactggctcctcctccaccaggcgaaccagagaataatagactctgtggctacaaggctgcatttcccaccagagagagtcatatcgaatttggctaattatggtaacacgagcgctgcttcgattccgctggctcttgatgaggcagtgagaagcggaaaagttaaaccaggacataccatagcgacatccggttttggagccggtttaacgtggggatcagcaattatgcgatggaggtgaatggctaagtccaacaatgtaagttaacttc

SEQ ID NO: 88 - DNA, 1881 bp, Arabidopsis thaliana
gaacataagctcttttcgcaaaacacacatcacacaccattttcacaacatcgtacttatcgccttcctctctctctcaatacctctctcaatttctggatccaccatgcaagctcttcaatcttcatctctccgtgcttctcctccaaacccacttcgcttaccatcaaatcgtcaatcacatcagctaattaccaatgcgagacctttgcgaagacaacaacgttccttcatctccgcatcagcatccactgtctccgctcctaaacgcgaaacagatccgaagaaacgagttgtcattactggtatgggtctcgtctctgtgtttggtaacgatgttgatgcttactacgagaaattgttgtctggtgagagtggaatcagtttgattgatcgtttcgatgcttccaagttccctactcgattcggtggtcagatccgtgggtttagctctgaaggttatattgatggcaagaatgagcgtaggcttgatgattgtttgaaatattgcattgttgctggtaaaaaagctcttgaaagtgccaatcttggtggtgataagcttaacacgattgataagaggaaagctggagtactagttgggactggaatgggaggtttaactgtgttttcagaaggtgttcagaatttgattgagaagggtcataggaggattagtccattttttataccttatgctataacaaatatgggttctgctttgttggcgattgatcttggtcttatgggtcctaactattcgatttcaactgcttgtgctacttcgaattactgcttttacgctgctgcgaatcacattcgtcgtggtgaagctgatatgatgattgctggtgggactgaggctgctattattcctattgggttgggaggttttgttgcttgtagggcattgtcccagagaaatgatgaccctcaaactgcttccaggccgtgggataaagcaagagatgggtttgttatgggtgaaggagctggtgttctggtgatggaaagcttggaacatgcaatgaaacgtggtgctccaattgtagcagaatatcttggaggtgctgttaattgtgatgctcaccatatgactgatccaagagctgatggtcttggggtttcttcatgcattgaaagatgcctggaagatgctggtgtatcacctgaggaggtaaattacatcaatgcacatgcaacttccactcttgctggtgatcttgctgagattaatgccattaaaaaggtattcaagagcacttcagggatcaaaatcaacgccaccaagtctatgataggtcactgcctcggtgcagctggaggtctagaagccatcgccaccgtgaaggctatcaacactggatggctgcatccttccatcaaccaatttaacccagaacaagctgtggactttgacacggtcccaaacgagaagaagcaacacgaggttgatgttgccatatcaaactcgttcgggttcggtggacacaactcggtagtcgccttctctgccttcaaaccctgatttcttcataccttttagattctctgccctatcggttactatcatcatccatcaccaccacttgcagcttcttggttcacaagttggagctcttcctctggccttttgcggttctttcattccccgtttcttacggttgctgagatttcagattttgtttgttctctctcttgtctgcggaatgttgtgtatcttagttcgttccatatttgcgtaatttataaaaacagaaactgagagaatcttgtagtaacggtgttattgtcagaataatccaattaggggattctcatcttttatttctcaacaattcttgtcgtgtttttacattcgaagaaattagatttatactg

SEQ ID NO: 89 - DNA, 17 bp, Artificial Sequence
  Synthetic oligonucleotide
cgttacgtatcggatcc

SEQ ID NO: 90 - DNA, 33 bp, Artificial Sequence
  Synthetic oligonucleotide
ctaggctcgagaagcttttacgccccgccctgc

SEQ ID NO: 91 - DNA, 34 bp, Artificial Sequence
  Synthetic oligonucleotide
aaatcgcatgcggttaaaccgaggggatgatgta

(19) United States
(12) Patent Application Publication
(10) Pub. No.: US 2009/0176272 A1
(43) Pub. Date: Jul. 9, 2009

(54) EXPRESSION OF NUCLEIC ACID SEQUENCES FOR PRODUCTION OF BIOFUELS AND OTHER PRODUCTS IN ALGAE AND CYANOBACTERIA

(75) Inventors: Michele M. Champagne, Honolulu, HI (US); Adelheid R. Kuehnle, Honolulu, HI (US)

Correspondence Address: KNOBBE MARTENS OLSON & BEAR LLP, 2040 MAIN STREET, FOURTEENTH FLOOR, IRVINE, CA, 92614, US

(73) Assignee: KUEHNLE AGROSYSTEMS, INC., Honolulu, HI (US)

(21) Appl. No.: 12/210,043

(22) Filed: Sep. 12, 2008

Related U.S. Application Data
(60) Provisional application No. 60/971,846, filed on Sep. 12, 2007.

(51) Int. Cl. C12P 21/00; C12N 15/82; C12P 21/02; C07H 21/04; C12N 15/63; C12N 5/10; C07H 1/08

(52) U.S. Cl. 435/69.1; 435/468; 536/23.1; 435/320.1; 435/419; 435/470; 536/25.4

(57) ABSTRACT

Various embodiments provide, for example, vectors, expression cassettes, and cells useful for transgenic expression of nucleic acid sequences. In various embodiments, vectors can contain plastid-based sequences of unicellular photosynthetic bioprocess organisms for the production of food- and feed-stuffs, oils, biofuels, pharmaceuticals or fine chemicals.

### REFERENCE TO RELATED APPLICATIONS

The present application claims priority to U.S. provisional application No. 60/971,846, filed Sep. 12, 2007, which is incorporated by reference herein.

### SEQUENCE LISTING

The present application is being filed along with a Sequence Listing in electronic format. The Sequence Listing is provided as a file entitled KAGRO_{—}001A.txt, created Sep. 12, 2008, which is 85.3 Kb in size. The information in the electronic format of the Sequence Listing is incorporated herein by reference in its entirety.

### BACKGROUND

The present invention pertains generally to expression of genes of interest in unicellular organisms. In particular, the invention relates to methods and compositions for targeted integration of expression constructs in chloroplasts of bioprocess marine algae and in clustered orthologous group loci in cyanobacteria.

Sequence requirements specific for chloroplast vectors for genetic engineering of the fresh-water green alga, Chlamydomonas, have been known since the 1980s. As was established in Chlamydomonas and subsequently well-illustrated in numerous higher plants, backbone vectors for targeted integration in plastid genomes preferably comprise flanking sequences that are host-specific. This is unlike vectors for nuclear transformation of algae and higher plants, in which site-directed integration of the nucleic acids is not required for expression and is uncommon and thus heterologous, non-host regulatory elements are frequently used. For proper functioning of encoded enzymes within the plastid compartment, a chloroplast transit peptide attached to the gene of interest can be included in vectors for nuclear transformation of eukaryotic algae and higher plants. Tissue specific promoters in vectors for nuclear transformation of higher plants can be used to express a gene of interest in, for example, seed tissue.

Cryptic sequences present in host plastid genomes may influence outcomes in transcription such that conservation of endogenous sequences in situ is desirable; conservation of such cryptic plastid sequences in heterologous vectors employed for plastidial targeted integration is not known. Thus, there is a need for algal transformation vectors comprised of host plastidial homologous flanking sequences for site-specific integration.

Nucleic acid uptake by plastids has been reported for the marine red microalga Porphyridium, but not for Dunaliella and Tetraselmis (Lapidot et al., Plant Physiol. 129: 7-12; 2002; Walker et al., J. Phycol. 41: 1077-1093; 2005). Lapidot et al. describe use of a native mutant gene used in a standard DNA plasmid vector backbone to produce a single cross-over event, randomly within the existing non-mutant gene. This results in integration of the entire vector along with reconstitution of both mutant and non-mutant loci for the gene of interest. This work does not teach use of dual flanking sequences with homology to the host genome for double cross-over events, nor does it teach use of a combination of homologous sequences with other elements for integration of the elements notably independent of the vector backbone. Moreover, this work does not enable use of a multitude of regulatory elements that can be used singly or in combination for de novo transplastomic algae, nor does it provide teachings on the genetic environment for integration and expression of other genes in cis with the integration site. The host red alga, Porphyridium, is not a recognized bioprocess algae. The commercially relevant algae amongst the Rhodophytes, i.e., red algae, are multicellular seaweeds, not unicellular microalgae, are taxonomically and evolutionarily distinct from green algae Chlorophytes, and are known to be useful for pigments and polyunsaturated fatty acids but not for biofuels.

Integration of nucleic acids in blue-green algae, i.e., cyanobacteria, can also proceed by homologous recombination, but use of integration vectors targeted to host cell loci coordinately involved in lipid metabolism has not been previously carried out. Some cyanobacteria such as Synechococcus can have a high fraction of saturated fatty acids compared to polyunsaturated fatty acids, which is highly desirable for oxidative stability of the oils, especially when used for biofuels. Since the total oil yields per unit weight of cyanobacteria are generally much lower than for other microalgae, increasing their capacity for fatty acid production by genetic manipulation is of keen interest.

Moreover, some cyanobacteria as well as eukaryotic algae can be grown as facultative heterotrophs such that they proliferate under illumination as well as under extended periods of darkness when fed organic carbon. Combining the ability to accelerate biomass production over time with methods to achieve higher overall isoprenoid and fatty acids biosynthesis by genetic transformation through homologous recombination is very attractive for a bioprocess organism.

### SUMMARY OF THE INVENTION

Various embodiments provide, for example, nucleic acids, polypeptides, vectors, expression cassettes, and cells useful for transgenic expression of nucleic acid sequences. In various embodiments, vectors can contain plastid-based sequences or clustered orthologous group sequences of unicellular photosynthetic bioprocess organisms for the production of food- and feed-stuffs, oils, biofuels, pharmaceuticals or fine chemicals.

In various embodiments, methods for producing a gene product of interest in marine algae is provided. The methods generally comprise: transforming a marine alga with a vector comprising a first chloroplast genome sequence, a second chloroplast genome sequence and a gene encoding a product of interest, wherein the gene is flanked by the first and second chloroplast genome sequences; and culturing the marine alga such that the gene product of interest is expressed. In some embodiments the gene product can be collected from the marine algae.

In some embodiments, the first and second chloroplast genome sequences each comprises at least about 300 contiguous base pairs of SEQ ID NO: 4.

In some embodiments, the gene product can be selected from the group consisting of IPP isomerase, acetyl-coA synthetase, pyruvate dehydrogenase, pyruvate decarboxylase, acetyl-coA carboxylase, α-carboxyltransferase, β-carboxyltransferase, biotin carboxylase, biotin carboxyl carrier protein and acyl-ACP thioesterase, beta ketoacyl-ACP synthase, FatB, and a protein that participates in fatty acid biosynthesis via the pyruvate dehydrogenase complex. In some embodiments, the gene product can be beta ketoacyl ACP synthase, and wherein the beta ketoacyl ACP synthase modifies fatty acid chain length in algae including cyanobacteria.

In some embodiments two or more genes encoding products of interest are expressed in the marine algae. For example, two or more gene products can be expressed coordinately in a polycistronic operon.

In various embodiments, plastid nucleic acid sequences for plastome recombination in unicellular bioprocess marine algae are provided. In some embodiments, a plastid nucleic acid sequence comprises SEQ ID NO: 4.

In various embodiments, vectors for targeted integration in the plastid genome of a unicellular bioprocess marine algae are provided. The vectors may comprise: a first segment of chloroplast genome sequence and a second segment of chloroplast genome sequence.

In some embodiments, the vector further comprises one or more genes of interest located between the first and second segments of chloroplast genome sequence. Preferably, the genes of interest do not interfere with production of gene products encoded by the first and second segments

In some embodiments, the gene of interest is operably linked to a transcriptional promoter provided by an operon of the targeted integration site.

In some embodiments, the first and second segments of chloroplast genome sequence each comprise at least 300 contiguous base pairs of SEQ ID NO: 4.

In some embodiments, unicellular bioprocess marine algae transformed with a vector are provided. The unicellular bioprocess marine algae typically comprise: a first segment of chloroplast genome sequence, a second segment of chloroplast genome sequence, and a gene or genes of interest, wherein the gene of interest is located between the first and second segments of chloroplast genome sequence. The bioprocess marine alga can be of the species Dunaliella or Tetraselmis.

In some embodiments, method of integrating a gene or genes of interest into the plastid genome of a unicellular bioprocess marine alga is provided. The methods comprise transforming a unicellular bioprocess marine alga with a vector comprising a first segment of chloroplast genome sequence, a second segment of chloroplast genome sequence, and a gene of interest, wherein the gene of interest is located between the first and second segments of chloroplast genome sequence.

In some embodiments, the transforming can be carried out using magnetophoresis, particularly moving pole magnetophoresis, electroporation, or a particle inflow gun.

In some embodiments, a method for isolation of a plastid nucleic acid from unicellular bioprocess marine algae for determination of contiguous plastid genome sequences is provided. The method comprises: passing the algae through a French press; isolating the chloroplasts using density gradient centrifugation; lysing the isolated chloroplasts; and isolating the plastid nucleic acid by density gradient centrifugation. The plastid nucleic acid can be a high molecular weight plastid nucleic acid. The unicellular bioprocess marine algae can be, for example, selected from the group consisting of Dunaliella and Tetraselmis.

In other embodiments, methods for producing one or more gene products of interest in cyanobacteria are provided. The methods generally comprise: transforming a cyanobacteria with a vector comprising a first clustered orthologous group sequence, a second clustered orthologous group sequence and a gene encoding a product of interest, wherein said gene is flanked by the first and second clustered orthologous group sequences; and culturing said cyanobacteria to produce the gene product. In some embodiments the gene product is collected from the cyanobacteria.

The first and second clustered orthologous group sequences may comprise, for example, at least 300 contiguous base pairs of SEQ ID NO: 70.

In some embodiments the gene product is selected from the group consisting of IPP isomerase, acetyl-coA synthetase, pyruvate dehydrogenase, pyruvate decarboxylase, acetyl-coA carboxylase, α-carboxyltransferase, β-carboxyltransferase, biotin carboxylase, biotin carboxyl carrier protein and acyl-ACP thioesterase, beta ketoacyl-ACP synthase, FatB, and a protein that participates in fatty acid biosynthesis via the pyruvate dehydrogenase complex.

In some embodiments the vector may comprise two or more genes encoding products of interest. The two or more genes may be expressed coordinately in a polycistronic operon.

In other embodiments, a vector for targeted integration in the genome of a cyanobacteria is provided, comprising a first segment of clustered orthologous group sequence and a second segment of clustered orthologous group sequence. The first and second segments of clustered orthologous group sequence may each comprise at least 300 contiguous base pairs of SEQ ID NO: 70.

The vector may also further comprising a gene of interest located between the first and second segments of clustered orthologous group sequence. Preferably, the gene of interest does not interfere with production of a gene product encoded by the first and second segments. The gene of interest may be operably linked to a transcriptional promoter from an operon of the targeted integration site.

In still other embodiments, cyanobacteria are provided that are transformed with a vector comprising a first segment of clustered orthologous group sequence, a second segment of clustered orthologous group sequence, and a gene of interest located between the first and second segments of clustered orthologous group sequence. The cyanobacteria may, for example, be of the species Synechocystis or Synechococcus.

In other embodiments methods of integrating a gene of interest into a clustered orthologous group of a cyanobacteria genome are provided. The methods typically comprise transforming a cyanobacteria with a vector comprising a first segment of clustered orthologous group sequence, a second segment of clustered orthologous group sequence, and a gene of interest, wherein said gene of interest is located between the first and second segments. Transformation may be carried out, for example, using prokaryotic conjugation or passive direct DNA uptake.

In another aspect of the invention, methods of transforming target cells, such as marine algae, by magnetophoresis are provided. Target cells are mixed with magnetizable particles, linearized transformation vector and carrier DNA. The mixture is then subject to a moving magnetic field, for example by placing the mixture on a spinning magnet such as a stir plate. The moving magnets penetrate the cells, delivering the transformation vector.

### BRIEF DESCRIPTION OF THE DRAWINGS

FIG. 1 depicts a map of a vector in accordance with some embodiments described herein.

FIG. 2 depicts a map of a vector in accordance with some embodiments described herein.

FIG. 3 depicts a map of a vector in accordance with some embodiments described herein.

FIG. 4 depicts a map of a vector in accordance with some embodiments described herein.

FIG. 5 depicts a map of a vector in accordance with some embodiments described herein.

FIG. 6 depicts a map of a vector in accordance with some embodiments described herein.

FIG. 7 depicts a map of a vector in accordance with some embodiments described herein.

FIG. 8 depicts a map of a vector in accordance with some embodiments described herein.

FIG. 9 depicts a map of a vector in accordance with some embodiments described herein.

FIG. 10 depicts a map of a vector in accordance with some embodiments described herein.

FIG. 11 depicts a map of a vector in accordance with some embodiments described herein.

FIG. 12 depicts a map of a vector in accordance with some embodiments described herein.

FIG. 13 depicts a map of a vector in accordance with some embodiments described herein.

FIG. 14 depicts a map of a vector in accordance with some embodiments described herein.

FIG. 15 depicts a map of a vector in accordance with some embodiments described herein.

FIG. 16 depicts a map of a vector in accordance with some embodiments described herein.

### DETAILED DESCRIPTION OF THE PREFERRED EMBODIMENT

Host-specific genomic and/or regulatory sequences can be used for expression of target genes in chloroplasts of bioprocess marine algae and in cyanobacteria. Some embodiments described herein provide methods for identifying and isolating contiguous chloroplast genome sequences or cyanobacterial clustered orthologous group sequences sufficient for designing and executing genetic engineering for unicellular photosynthetic bioprocess marine algae and cyanobacteria. Once these fundamental sequences are discovered, further modifications may be made for purposes of optimized expression. Thus, various other embodiments described herein provide methods for transgenic expression of nucleic acid sequences in unicellular organisms such as bioprocess marine algae and cyanobacteria, as well as various nucleic acids, polypeptides, vectors, expression cassettes, and cells useful in the methods.

Until now, no contiguous chloroplast genome sequences sufficient for designing and executing plastid genetic engineering have been reported for unicellular photosynthetic bioprocess marine algae. Further, associated methods for application of such vectors are unreported. Bioprocess algae are those that are scaleable and commercially viable. Two target well-known bioprocess microalgae are Dunaliella and Tetraselmis. The former is recognized for its use in producing carotenoids and glycerol for fine chemicals, foodstuff additives, and dietary supplements, the latter in aquaculture feed. Carbon metabolism in the algae is relevant for all these products, with the chloroplast being the initial site for all isoprenoid and fatty acid metabolism. More recently interest in algae biomass for biofuels feedstock and the associated carbon dioxide and nitrous oxide sequestration has emerged (Christi, Biotechnology Advances 25: 294-306; 2007; Huntley M E and D G Redalje, Mitigation and Adaptation Strategies for Global Change 12: 573-608; 2007).

In some embodiments, methods are provided for isolation of high molecular weight plastid nucleic acids from bioprocess marine algae. As discussed above, until now, no contiguous chloroplast genome sequences sufficient for designing and executing plastid genetic engineering have been reported for unicellular photosynthetic bioprocess marine algae. In various embodiments, plastid nucleic acids from unicellular bioprocess marine algae can be used for identification of contiguous plastid genome sequences sufficient for designing integrating plastid nucleic acid constructs, and gene expression cassettes thereof. In some embodiments, methods are provided for obtaining specific sequences of the marine algal chloroplast genome and in other embodiments methods of obtaining specific sequences from cyanobacteria. Also disclosed are plastid nucleic acid sequences useful for targeted integration into marine algae plastids as well as nucleic acid sequences useful for targeted integration in cyanobacteria. Exemplary marine algae include without limitation Dunaliella and Tetraselmis.

Some embodiments provide expression vectors for the targeted integration and expression of genes in marine algae and cyanobacteria. In various embodiments, methods are provided for transformation of expression vectors into marine algae chloroplasts and their evolutionary ancestors, cyanobacteria. In some embodiments, methods are provided for targeted integration of one or more genes into the marine algae chloroplast and cyanobacteria genomes. In other embodiments, methods are provided for the expression of genes that have been integrated into the chloroplast or cyanobacteria genomes. In some embodiments, the genes can be, for example, genes that aid in selection, such as genes that participate in antibiotic resistance. In other embodiments, the genes can be, for example, genes that participate in, or otherwise modulate, carbon metabolism, such as in isoprenoid and fatty acid biosynthesis. In some embodiments, multiple genes are present.

### SOME DEFINITIONS

Unless defined otherwise, all technical and scientific terms used herein have the meaning commonly understood by a person skilled in the art to which this invention belongs. As used herein, the following terms have the meanings ascribed to them unless specified otherwise.

The articles “a” and “an” are used herein to refer to one or to more than one (i.e., to at least one) of the grammatical object of the article. By way of example, “an element” means one element or more than one element.

By “expression vector” is meant a vector that permits the expression of a polynucleotide inside a cell and/or plastid. Expression of a polynucleotide includes transcriptional and/or post-transcriptional events. An “expression construct” is an expression vector into which a nucleotide sequence of interest has been inserted in a manner so as to be positioned to be operably linked to the expression sequences present in the expression vector.

The phrase “expression cassette” refers to a complete unit of gene expression and regulation, including structural genes and regulating DNA sequences recognized by regulator gene products.

By “plasmid” is meant a circular nucleic acid vector. Plasmids contain an origin of replication that allows many copies of the plasmid to be produced in a bacterial (or sometimes eukaryotic) cell without integration of the plasmid into the host cell DNA.

The term “gene” as used herein refers to any and all discrete coding regions of a host genome, or regions that code for a functional RNA only (e.g., tRNA, rRNA, regulatory RNAs such as ribozymes etc). The gene can include associated non-coding regions and optionally regulatory regions. In certain embodiments, the term “gene” includes within its scope the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. In some embodiments the gene sequences may be cDNA or genomic DNA or a fragment thereof. The gene may be introduced into an appropriate vector for extrachromosomal maintenance or for integration into the host.

The term “control sequences” or “regulatory sequence” as used herein refers to nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

By “operably connected” or “operably linked” and the like is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous and, where necessary to join two protein coding regions, contiguous and in reading frame. A coding sequence is “operably linked to” another coding sequence when RNA polymerase will transcribe the two coding sequences into a single mRNA, which is then translated into a single polypeptide having amino acids derived from both coding sequences. The coding sequences need not be contiguous to one another so long as the expressed sequences are ultimately processed to produce the desired protein. “Operably connecting” a promoter to a transcribable polynucleotide is meant placing the transcribable polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the transcribable polynucleotide, which is approximately the same as the distance between that promoter and the gene it controls in its natural setting; i.e.: the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e. the genes from which it is derived.

The term “promoter” as used herein refers to a minimal nucleic acid sequence sufficient to direct transcription of a DNA sequence to which it is operably linked. The term “promoter” is also meant to encompass those promoter elements sufficient for promoter-dependent gene expression. Promoters may be used, for example, for cell-type specific expression, tissue-specific expression, or expression induced by external signals or agents. Promoters may be located 5′ or 3′ of the gene to be expressed.

The term “inducible promoter” as used herein refers to a promoter that is transcriptionally active when bound to a transcriptional activator, which in turn is activated under a specific condition(s), e.g., in the presence of a particular chemical signal or combination of chemical signals that affect binding of the transcriptional activator to the inducible promoter and/or affect function of the transcriptional activator itself.

By “construct” is meant a recombinant nucleotide sequence, generally a recombinant nucleic acid molecule that has been generated for the purpose of the expression of a specific nucleotide sequence(s), or is to be used in the construction of other recombinant nucleotide sequences. In general, “construct” is used herein to refer to a recombinant nucleic acid molecule.

The term “transformation” as used herein refers to a permanent or transient genetic change, preferably a permanent genetic change, induced in a cell following incorporation of one or more nucleic acid sequences. Where the cell is a plant cell, a permanent genetic change is generally achieved by introduction of the nucleic acid into the genome of the cell, and specifically into the plastome (plastid genome) of the cell for plastid-encoded genetic change.

The term “host cell” as used herein refers to a cell that is to be transformed using the methods and compositions of the invention. Transformation may be designed to non-selectively or selectively transform the host cell(s). Host cells may be prokaryotes or eukaryotes. In general, host cell as used herein means a marine algal cell or cyanobacterial cell into which a nucleic acid of interest is transformed.

The term “transformed cell” as used herein refers to a cell into which (or into an ancestor of which) has been introduced, by means of recombinant nucleic acid techniques, a nucleic acid molecule. The nucleic acid molecule typically encodes a gene product (e.g., RNA and/or protein) of interest (e.g., nucleic acid encoding a cellular product).

The term “gene of interest,” “nucleotide sequence of interest,” “nucleic acid of interest” or “DNA of interest” as used herein refers to any nucleic acid sequence that encodes a protein or other molecule that is desirable for expression in a host cell (e.g., for production of the protein or other biological molecule (e.g., an RNA product) in the target cell). The nucleotide sequence of interest is generally operatively linked to other sequences which are needed for its expression, e.g., a promoter. It is well-known in the art that the degeneracy of the DNA code allows for more than one triplet combination of DNA base pairs to specify a particular amino acid. When a nucleic acid sequence is to be expressed in a non-host cell, the use of host-preferred codons is desirable. The sources of genes of interest is not limited and may be, for example, prokaryotes, eukaryotes, algae, cyanobacteria, bacteria, plants, and viruses.

“Culturing” signifies incubating a cell or organism under conditions wherein the cell or organism can carry out some, if not all, biological processes. For example, a cell that is cultured may be growing or reproducing, or it may be non-viable but still capable of carrying out biological and/or biochemical processes such as replication, transcription, translation, etc.

By “transgenic organism” is meant a non-human organism (e.g., single-cell organisms (e.g., microalgae), mammal, non-mammal (e.g., nematode or Drosophila)) having a non-endogenous (i.e., heterologous) nucleic acid sequence present in a portion of its cells or stably integrated into its germ line DNA.

The term “biomass,” as used herein refers to a mass of living or biological material and includes both natural and processed, as well as natural organic materials more broadly.

The term “unicellular” as used herein refers to a cell that exists and reproduces as a single cell. Many algae and cyanobacteria exist as unicellular organisms that can be free-living single cells or colonial. The distinction between a colonial organism and a multicellular organism is that individual organisms from a colony can survive on their own in their natural environment if separated from the colony, whereas single cells from a multicellular organism cannot survive in their natural environment if separated.

For hydrocarbon chain length, “short” chains are those with less than 8 carbons; “medium” chains are inclusive of 8 to 14 carbons; and “long” chains are those with 16 carbons or more.

### Preparation of Marine Algae Plastid DNA

Some of the presently disclosed embodiments are directed to methods for preparation of marine algal DNA. High molecular weight plastid nucleic acids from unicellular bioprocess marine algae can be used, for example, for identification of contiguous plastid genome sequences sufficient for designing integrating plastid nucleic acid constructs. In some embodiments, the methods provide DNA as purified fractions of nuclear, chloroplast and mitochondrial origin. As described in detail below, some of the methods involve isolation of the chloroplasts using a French press, and subsequent purification of the DNA by density gradient centrifugation.

In some embodiments, methods for preparation of marine algae DNA comprise passing the algae through a French press and using density gradient centrifugation to isolate the chloroplasts. The isolated chloroplasts can then be lysed, and the plastid DNA can be isolated by, for example, density gradient centrifugation. After density gradient centrifugation, the plastid DNA can be extracted and dialyzed. Subsequently, the plastid DNA can be precipitated. The precipitated DNA can be further purified, such as, for example, by chloroform extraction. The purified DNA is suitable for a variety of procedures, including, for example, sequencing.

In various embodiments, marine algae can be grown in media for the preparation of plastid DNA. A variety of media and growth conditions for marine algae are known in the art. (Andersen, R. A. ed. Algal Culturing Techniques. Psychological Society of America, Elsevier Academic Press; 2005). For example, in various embodiments, the algae may be grown in medium containing about 1 M NaCl at about room temperature (20-25° C.). In some embodiments, the marine algae can be grown under illumination with white fluorescent light (for example, about 80 umol/m²sec) with, for example, about a 12 hour light: 12 hour dark photoperiod. The volume of growth medium may vary. In some embodiments, the volume of media can be between about 1 L to about 100 L. In some embodiments, the volume is between about 1 L to about 10 L. In some embodiments, the volume is about 4 L.

Algal cells of growth by can be collected in the late logarithmic phase centrifugation. The cell pellet can be washed to remove cell surface materials which may cause clumping of cells.

After collection of the algal cells, the cell pellet can be resuspended isolation medium. The isolation medium is typically cold. In some embodiments, the isolation medium is ice-cold. A variety of different buffers may be used as isolation media (Andersen, R. A. ed. Algal Culturing Techniques. Psychological Society of America, Elsevier Academic Press; 2005). In some embodiments, the isolation medium can comprise, for example, about 330 mM sorbitol, about 50 mM HEPES, about 3 mM NaCl, about 4 mM MgCl₂, about 1 mM MnCl₂, about 2 mM EDTA, about 2 mM DTT, about 1 mL/L proteinase inhibitor cocktail. In some embodiments, the cell pellet can be resuspended to a concentration equivalent to, for example, about 1 mg chlorophyll per mL of isolation medium.

The chlorophyll concentration may be estimated by a variety of methods known by those of skill in the art. For example, chlorophyll concentration may be estimated by adding 10 uL of the chloroplast suspension to 1 mL of an 80% acetone solution and mixing well. The solution is centrifuged for about 2 min at, for example, about 3000×g. The absorbance of the supernatant is measured at 652 nm using the 80% acetone solution as the reference blank. The absorbance is multiplied by the dilution factor (100) and divided by the extinction coefficient of 36 to determine the mg of chlorophyll per mL of the chloroplast suspension. The solution is adjusted to a concentration of 1 mg chlorophyll per mL with additional cold isolation medium.

In various embodiments, the resultant cell suspension in the isolation medium can be placed for about 2 min in, for example, a French press at between about 300 to about 5000 pounds per square inch (psi). The pressure of the French press can be set at a pressure determined to be ideal for the species, ranging from about 300 psi to about 5000 psi. In some embodiments, the pressure of the French press is about 700 psi. In other embodiments pressure of the French press is between about 3000 to about 5000 psi. Preferably, the French press is cold. In some embodiments, the French press is ice-cold. The outlet valve of the French press can then be opened, for example, to a flow rate of about 2 mL/min, and the pressate can be collected in a tube containing an equal volume of isolation medium. The collection tube can be chilled and the isolation medium can be ice-cold. In some embodiments the intact chloroplasts from the pressate can be collected as a loose pellet by, for example, centrifugation at about 1000×g for about 5 minutes.

After a subsequent washing step, density centrifugation can be used to isolate the chloroplasts. Various methods for density gradient separation are known in the art. In some embodiments, the pellet can be resuspended in, for example, about 3 mL of isolation medium per liter of starter culture and loaded on the top of a 30 mL discontinuous gradient of, for example, 20, 45, and 65% Percoll in 330 mM sorbitol and 25 mM HEPES-KOH (pH 7.5). The density gradient conditions can vary. Density centrifugation can be carried out in, for example, a swinging bucket rotor with slow acceleration at about 1000×g for about 10 mins, then at about 4000×g for about another 10 min, and then slow deceleration. Centrifugation conditions can vary. The intact chloroplasts in the 20-45% Percoll interphase can be collected with, for example, a plastic pipette. To remove the Percoll, the chloroplast suspension can be diluted about 10-fold with isolation medium and the chloroplasts can be pelleted by centrifugation about 1000×g for about 2 min. In some embodiments, the washing step can be repeated once. Washed chloroplasts can then be resuspended in a small volume of, for example, isolation medium to a chlorophyll concentration of approximately 1 mg/mL.

A variety of methods can be used to lyse the isolated plastids. For example, in some embodiments, the plastids can be lysed by the addition of an equal volume of lysis buffer containing, for example, about 50 mM Tris (pH 8), about 100 mM EDTA, about 50 mM NaCl, about 0.5% (w/v) SDS, about 0.7% (w/v) N-lauroyl-sarcosine, about 200 ug/mL proteinase K, and about 100 ug/mL RNAse. The solution can be mixed by inversion and incubated for about 12 hours at about 25° C. Lysis of the plastids can be confirmed by, for example, microscopic examination.

The lysate from the plastids can then be separated using a density gradient. In some embodiments, the lysate is separated using a CsCl density gradient. For example, the solution containing plastid DNA can be transferred to a tube and ultrapure CsCl added to a concentration of about 1 g/mL. The solution can be centrifuged at about 27,000×g at about 20° C. for about 30 min in, for example, a SW41 swing-out rotor using Beckman #331372 ultracentrifuge tubes. For example, the cleared lysate can be collected and transferred to a tube, diluted with water to about 0.7-0.8 g/mL CsCl and transferred to, for example, polyallomer ultracentrifuge tubes. Dye, such as, for example, Hoechst 33258 DNA-binding fluorescent dye, can be added to fill the centrifuge tube to the desired concentration. The tube can filled to maximum with additional 0.8 g/mL CsCl in TE buffer or deionized distilled water, (mass 1.60 to 1.69 g/mL). The sample is centrifuged at, for example, about 190,000×g (about 44,300 rpm) at about 20° C. for about 48 hours in, for example, a VTi50 fixed-angle rotor. Chloroplast DNA can be visualized in the resulting gradient using, for example, a long-wave UV lamp, and the DNA can be removed from the gradient with an 18-gauge needle and syringe. The dye (e.g., Hoechst 33258) can be removed by, for example, repeated extractions with, for example, 2-propanol saturated with 3 M NaCl. A UV lamp may be used to verify complete removal of the dye. The CsCl concentration can be reduced by, for example, overnight dialysis (e.g., Pierce Slide-A-Lyzer 10,000 mwco) against three changes of TE buffer.

The isolated plastid DNA can then be precipitated. A variety of methods for DNA precipitation are well-known in the art. For example, DNA can be precipitated with about 2.5 volumes of 2-propanol plus about 0.1 volume of about 3 M sodium acetate (pH 5.2) followed by incubation at −20° C. for about 1 hour. The solution can be transferred to centrifuge tubes and spun, for example, at about 18,000×g, 4° C. for about 2 hours. The chloroplast DNA pellet can be dried at room temperature and resuspended in, for example, about 1 mL TE. In some embodiments, the solution can be further purified by extracting three times with, for example, phenol-chloroform-isoamyl alcohol (24:24:1) and twice with chloroform-isoamyl alcohol (24:1), mixing by inversion and centrifuging at about 1000×g for about 10 minutes after each extraction. A second 2-propanol precipitation can be performed. The DNA pellet can be washed with, for example, 70% ethanol, dried, and resuspended in TE buffer. The resulting DNA solution can be quantified by, for example, optical density at 260 nm.

By the above method DNA can be recovered as purified fractions of nuclear, chloroplast and mitochondrial origin. While the procedure enriches for chloroplasts, nuclear and mitochondrial nucleic acids are present as well and are removed during the ultracentrifugation and fraction isolation from CsCl gradient. From top to bottom on the cesium chloride gradient, distinct bands of DNA migrate based upon mass, with mitochondrial DNA at top, chloroplast DNA in the middle and nuclear DNA at the bottom of the gradient. The yield of DNA may vary. In some embodiments, yield of DNA per liter of culture at, for example, about 2×10⁶ cells/m¹ can be about 0.9 μg chloroplast DNA and about 2.0 μg nuclear DNA.

### Sequencing of Plastid DNA

Plastid DNA can be sequenced by any of a variety of methods known in the art. In some embodiments, plastid DNA can be sequenced using, for example without limitation, shotgun sequencing or chromosome walking techniques. In various embodiments, shotgun genome sequencing can be performed by cloning the chloroplast DNA into, for example, pCR4 TOPO® blunt shotgun cloning kit according to the manufacturer's instructions (Invitrogen). In various embodiments, shotgun clones can be sequenced from both ends using, for example, T7 and T3 oligonucleotide primers and a KB basecaller integrated with an ABI 3730XL® sequencer (Applied Biosystems, Foster City, Calif.). Sequences can be trimmed to remove the vector sequences and low quality sequences, then assembled into contigs using, for example, the SeqMan II® software (DNAStar). Plastid DNA can be sequenced by a number of different methods known in the art for sequencing DNA.

Sequence information obtained from sequencing the plastid DNA can be analyzed using a variety of methods, including, for example, a variety of different software programs. For example, contigs can be processed to identify coding regions using, for example, the Glimmer® software program. ORFs (open reading frames) can be saved, for example, in both nucleotide and amino acid sequence Fasta formats. Any putative ORFs can be searched against the latest Non-redundant (NR) database from NCBI using the BLASTP program to determine similarity to known protein sequences in the database.

### Vectors

Nucleic acid vectors are used for targeted integration into the chloroplast genome or cyanobacteria genome. In various embodiments, one or more genes of interest can be introduced and expressed in a host cell via a chloroplast or orthologous gene group. The vectors typically comprise a vector backbone, one or more chloroplast or orthologous gene group genomic sequences and an expression cassette comprising the gene or genes of interest.

In various embodiments, plastid nucleic acid vectors comprising chloroplast nucleic acid sequences are used to target integration into the chloroplast genome. The plastid nucleic acid vectors comprise one or more genes of interest to be integrated into the chloroplast genome and expressed by the marine algae. In some embodiments, integration is targeted such that the gene of interest does not interfere with expression of gene products in the host.

In other embodiments nucleic acid vectors comprise one or more cyanobacteria genomic sequences and one or more genes of interest to be expressed in the cyanobacteria. The vectors thus target integration of the gene or genes of interest into the cyanobacteria genome. Preferably, such integration does not interfere with expression of gene products in the host.

In some embodiments, the vectors comprise a gene expression cassette. The gene expression cassette may comprise one or more genes of interest, as discussed in greater detail below, that are to be integrated into the chloroplast genome or the cyanobacteria genome and expressed. The expression cassettes may also comprise one or more regulatory elements, such as a promoter operably linked to the gene of interest. In some embodiments the gene of interest is operably linked to a transcriptional promoter from an operon of the targeted integration site.

Standard molecular biology techniques known to those skilled in the art of recombinant nucleic acid and cloning can be used to prepare the vectors and expression cassettes unless otherwise specified. For example, the various fragments comprising the various constructs, expression cassettes, markers, and the like may be introduced consecutively by restriction enzyme cleavage of an appropriate replication system, and insertion of the particular construct or fragment into the available site. After ligation and cloning the vector may be isolated for further manipulation. All of these techniques are amply exemplified in the literature and find particular exemplification in Maniatis et al., Molecular cloning: a laboratory manual, 3^{rd} ed. (2001) Cold Spring Harbor Laboratory Press, Cold Spring Harbor, N.Y.

In developing the constructs the various fragments comprising the regulatory regions and open reading frame may be subjected to different processing conditions, such as ligation, restriction enzyme digestion, PCR, in vitro mutagenesis, linkers and adapters addition, and the like. Thus, nucleotide transitions, transversions, insertions, deletions, or the like, may be performed on the nucleic acid which is employed in the regulatory regions or the nucleic acid sequences of interest for expression in the plastids. Methods for restriction digests, Klenow blunt end treatments, ligations, and the like are well known to those in the art and are described, for example, by Maniatis et al.

During the preparation of the constructs, the various fragments of nucleic acid can be cloned in an appropriate cloning vector, which allows for amplification of the nucleic acid, modification of the nucleic acid or manipulation of the nucleic acid by joining or removing sequences, linkers, or the like. In some embodiments, the vectors will be capable of replication to at least a relatively high copy number in E. coli. A number of vectors are readily available for cloning, including such vectors as pBR322, vectors of the pUC series, the M13 series vectors, and pBluescript vectors (Stratagene; La Jolla, Calif.).

Chloroplast genomic sequences can be analyzed to identify chloroplast genomic sequence segments useful for targeted integration into the chloroplast genome (Maliga P., Annu. Rev. Plant Biol. 55:289-313; 2004). Generally, plastic vectors comprise segments of chloroplast genomic DNA sequence flanking both sides of a nucleic acid of interest that is to be integrated into the plastid genome. Similarly, vectors for integration into the cyanobacteria genome comprise segments of genomic cyanobacteria DNA flanking the nucleic acid of interest. The genomic DNA flanking sequences are preferably selected such that integration of the gene of interest does not interfere significantly with production of gene products encoded by the genomic sequences.

For example, a construct can comprise a first flanking genomic DNA segment, a second genomic DNA segment, and a nucleic acid of interest between the first and second genomic DNA segments. In some embodiments, the first and second genomic sequences are derived from a single, contiguous genomic sequence. A double recombination event will integrate the nucleic acid of interest. In some embodiments, the flanking pieces can be from about 1 kb to about 2 kb in length. In other embodiments each of the first and second genomic nucleic acid segments are preferably at least about 300 bases in length. In some embodiments the first and second flanking pieces each comprise at least about 300 bases of SEQ ID NO:4 (described below). The two flanking pieces may be a continuous sequence that is separated by the gene of interest.

A non-flanking piece of chloroplast DNA can direct integration by only a single recombination event. Thus, in other embodiments, the vector comprises a single genomic sequence. The single genomic sequence may be contiguous with the gene of interest. Preferably the single genomic sequence is at least about 300 bp in length.

A genomic DNA segment for targeted integration can be from about ten nucleotides to about 20,000 nucleotides long. In some embodiments, a genomic DNA segment for targeted integration can be about can be from about 300 to about 10,000 nucleotides long. In other embodiments, a genomic DNA segment for targeted integration is between about 1 kb to about 2 kb long. In some embodiments, a “contiguous” piece of genomic DNA is split into two flanking pieces on either side of a gene of interest. In some embodiments, the gene of interest is cloned into a non-coding region of a contiguous genomic sequence. In other embodiments, two genomic nucleic acid segments flanking a gene of interest comprise segments of genomic sequence which are not contiguous with one another in the wild type genome. In some embodiments, a first flanking genomic DNA segment is located between about 0 to about 10,000 base pairs away from a second flanking genomic DNA segment in the chloroplast genome.

The expression vector can comprise one or more genes that are desired to be expressed in the marine algae or cyanobacteria. In some embodiments a selectable marker gene and at least one other gene of interest are used. Genes of interest are described in more detail below.

The genomic nucleic acid segments and the nucleic acid encoding the gene of interest are introduced into a vector to generate a backbone expression vector for targeted integration of the gene of interest into a chloroplast or cyanobacteria genome. Any of a variety of methods known in the art for introducing nucleic acid sequences can be used. For example, nucleic acid segments can be amplified from isolated chloroplast or cyanobacteria genomic DNA using appropriate primers and PCR. The amplified products can then be introduced into any of a variety of suitable cloning vectors by, for example, ligation. Some useful vectors include, for example without limitation, pGEM13z, pGEMT and pGEMTEasy (Promega, Madison, Wis.); pSTBlue1 (EMD Chemicals Inc. San Diego, Calif.); and pcDNA3.1, pCR4-TOPO, pCR-TOPO-II, pCRBlunt-II-TOPO (Invitrogen, Carlsbad, Calif.). In some embodiments, at least one nucleic acid segment from a chloroplast is introduced into a vector. In other embodiments, two or more nucleic acid segments from a chloroplast or cyanobacteria genome are introduced into a vector. In some embodiments, the two nucleic acid segments can be adjacent to one another in the vector. In some embodiments, the two nucleic acid segments introduced into a vector can be separated by, for example, between about one and thirty base pairs. In some embodiments, the sequences separating the two nucleic acid segments can contain at least one restriction endonuclease recognition site.

In various embodiments, regulatory sequences can be included in the vectors of the present invention. In some embodiments, the regulatory sequences comprise nucleic acid sequences for regulating expression of genes (e.g., a nucleic acid of interest) introduced into the chloroplast genome. In various embodiments, the regulatory sequences can be introduced into a backbone expression vector, such as in. For example, various regulatory sequences can be identified from the marine algal chloroplast genome. One or more of these regulator sequences can be utilized to control expression of a gene of interest integrated into the chloroplast genome. The regulatory sequences can comprise, for example, a promoter, an enhancer, an intron, an exon, a 5′ UTR, a 3′ UTR, or any portions thereof of any of the foregoing, of a chloroplast gene. In other embodiments regulatory elements from cyanobacteria are used to control expression of a gene integrated into a cyanobacteria genome. In other embodiments, regulatory elements from other organisms are utilized. Using standard molecular biology techniques, the regulatory sequences can be introduced the desired vector. In some embodiments, the vectors comprise a cloning vector or a vector comprising nucleic acid segments for targeted integration. Recognition sequences for restriction enzymes can be engineered to be present adjacent to the ends of the regulatory sequences. The recognition sequences for restriction enzymes can be used to facilitate introduction of the regulatory sequence into the vector.

In some embodiments, nucleic acid sequences for regulating expression of genes introduced into the chloroplast genome can be introduced into a vector by PCR amplification of a 5′ UTR, 3′ UTR, a promoter and/or an enhancer, or portion thereof. Using suitable PCR cycling conditions, primers flanking the sequences to be amplified are used to amplify the regulatory sequences. In some embodiments, the primers can include recognition sequences for any of a variety of restriction enzymes, thereby introducing those recognition sequences into the PCR amplification products. The PCR product can be digested with the appropriate restriction enzymes and introduced into the corresponding sites of a vector.

In some embodiments, selection of transplastomic algae or transfected cyanobacteria can be facilitated by a selectable marker, such as resistance to antibiotics. Thus, in some embodiments, the vectors can comprise at least one antibiotic resistance gene. The antibiotic resistance gene can be any gene encoding resistance to any antibiotic, including without limitation, phleomycin, spectinomycin, kanamycin, chloramphenicol, hygromycin and any analogues. Other selectable markers are know in the art and can readily be employed.

Plastid nucleic acid vectors and/or cyanobacteria vectors may comprise a gene expression cassette comprising a gene of interest operably linked to a one or more regulatory elements. In some embodiments a gene expression cassette comprises one or more genes of interest operably linked to a promoter. Promoters that can be used include, for example without limitation, a psbA promoter, a psbD promoter, an atpB promoter, and atpA promoter, a Prrn promoter, a clpP protease promoter, and other promoter sequences known in the art, such as those described in, for example, U.S. Pat. No. 6,472,586, which is incorporated herein by reference in its entirety. In some embodiments, the gene expression cassette is present in the plastid nucleic acid vector adjacent to one or more chloroplast DNA sequence segments useful for targeted integration into the chloroplast genome. In some embodiments, the gene expression cassette is present in the plastid nucleic acid vector between two chloroplast DNA sequence segments. Similarly, in some embodiments the gene expression cassette is present in the cyanobacteria nucleic acid vector adjacent to one or more cyanobacteria genomic sequence segments useful for targeted integration into the cyanobacteria genome. In some embodiments, the gene expression cassette is present in the cyanobacteria nucleic acid vector between two cyanobacteria genomic sequence segments.

As referred to above, some of the presently disclosed embodiments are directed to the discovery of targeted integration into a cyanobacterial cluster of orthologous groups. In some embodiments, cyanobacteria vectors contain sequences that allow replication of the plasmid in Escherichia coli, nucleic acid sequences that are derived from the genome of the cyanobacteria, and additional nucleic acid sequences of interest such as those described in more detail below. It is known in the art that transformation frequencies of approximately 5×10⁻³ per colony forming units can be obtained in cyanobacteria if the transforming plasmid excludes nucleic acid sequences that allow replication in the cyanobacteria host cell, thereby promoting homologous recombination into the genome of the host cell (Tsinoremas et al., J. Bacteriol. 176(21): 6764-8; 1994). Thus, in some embodiments, nucleic acids that allow replication in cyanobacteria are omitted. This method is preferred over the method in which the plasmid is able to replicate in the cyanobacteria host cell, where transformation frequencies are reduced to approximately 10⁻⁵ per colony forming units (Golden S S and L A Sherman, J. Bacteriol. 155(3): 966-72; 1983).

Prokaryotic genomes arrange genes of related function adjacent to one another in operons, such that all members of the operon are co-expressed transcriptionally. This allows for efficient co-regulation of genes that comprise multisubunit protein complexes or act upon substrates that are intermediates of a common metabolic pathway. This operon organization of genes may be conserved between phylogenetically distant species at a low frequency because an entire operon tends to be selected over individual genes during a horizontal transfer event (Lawrence J G and J R Roth, Genetics, 143:1843-1860; 1996). Additionally, the ‘superoperon’ concept (Lathe et al., Trends Biochem. Sci. 25:474-479; 2000) has been proposed to describe the phenomenon whereby operons for genes with related functions are inherited as ‘neighborhoods’. The archetypical and largest superoperon is that for genes participating in translation and transcription (Rogozin et al., Nucleic Acids Res. 30(10):2212-2223; 2002). A second-ranked example is that for genes participating in lipid metabolism and amino acid metabolism.

Sequencing of complete bacterial genomes has demonstrated that operons are subject to multiple rearrangements over evolutionary time (Watanabe et al., J. Mol. Evol. 44:S57-S64; 1997). Genome comparisons by diagonal plots of distantly-related species reveal orthologous genes, but by one survey, as few as 5 to 25% of genes are identified in probable operons with an identical gene order in two or more genomes (Wolf et al., Genome Res. 11:356-372; 2001). Therefore, due to the low degree of gene order conservation, there is no single genomic locus suitable for design of a homologous recombination-based transformation vector applicable to all prokaryotes.

Analysis of cyanobacterial orthologous groups (CyOGs) was performed by Mulkidjanian et al. (2006) for 15 cyanobacterial genomes for which complete sequence data are available. The authors identified a core set of 892 genes present in all cyanobacterial genomes, and a subset of 84 of these that are shared exclusively with plants, including red algae and diatoms.

An additional set of CyOGs were identified as being uniquely shared with plastid-bearing eukaryotes but missing in other eukaryotes. This set includes genes for the deoxyxylulose pathway of terpenoid biosynthesis and fatty acid biosynthesis. This number two ranked cyanobacterial cluster of orthologous groups, which contains mostly genes for lipid and amino acid metabolism, comprise an ideal target locus for the development of cyanobacteria-specific transformation vectors. Thus, in some embodiments, one or more genomic sequences from this set of CyOGs are used to direct integration of one or more genes of interest into this orthologous cluster. In some embodiments, genomic DNA sequences from Synechocystis sp PCC6803 are used. For example, a first genomic sequence comprising at least 300 bases of SEQ ID NO: 70 and a second genomic sequence comprising at least about 300 bases of SEQ ID NO: 70 may be used. A gene of interest is preferably inserted between the two sequences.

### Transformation and Expression

In various embodiments, the plastid nucleic acid vectors can be introduced, or transformed, into marine algae chloroplasts or into cyanobacteria. Genetic engineering techniques known to those skilled in the art of transformation can be applied to carry out the methods using baseline principles and protocols unless otherwise specified.

A variety of different kinds of marine algae can be used as hosts for transformation with the vectors disclosed herein. In some embodiments, the marine algae can be Dunaliella or Tetraselnis. In other embodiments other algae and blue-green algae that can be used may include, for example, one or more algae selected from Acaryochloris, Amphora, Anabaena, Anacystis, Anikstrodesmis, Botryococcus, Chaetoceros, Chlorella, Chlorococcum, Crocosphaera, Cyanotheca, Cyclotella, Cylindrotheca, Euglena, Hematococcus, Isochrysis, Lyngbya, Microcystis, Monochrysis, Monoraphidium, Nannochloris, Nannochloropsis, Navicula, Nephrochloris, Nephroselmis, Nitzschia, Nodularia, Nostoc, Oochromonas, Oocystis, Oscillartoria, Pavlova, Phaeodactylum, Platymonas, Pleurochrysis, Porhyra, Prochlorococcus, Pseudoanabaena, Pyramimonas, Selenastrum, Stichococcus, Synechococcus, Synchocystis, Thalassiosira, Thermosynechocystis, and Trichodesmium.

Cyanobacteria can also be used as hosts for transformation with vectors described herein. Cyanobacteria suitable for use in the present invention include, for example without limitation, wild type Synechocystis sp. PCC 6803 and a mutant Synechocystis created by Howitt et al. (1999) that lacks a functional NDH type 2 dehydrogenase (NDH-2(−)).

While the utility of the invention may have broadest applicability to marine species, one or more of above organisms are also suited to growth in non-saline conditions, either naturally or through adaptation or mutagenesis, and thus this invention is not restricted to natural marine organisms. Further, one or more of the above organisms can be grown with supplemental organic carbon, including under darkness. Therefore, in various embodiments, the vectors can be introduced into algae and cyanobacteria organisms grown in, for example without limitation, fresh water, salt water, or brine water, with additional organic carbon for proliferation under darkness or alternating darkness and illumination. In another embodiment, the hydrocarbon composition and yields of one or more of the above organisms can be modulated by their culture conditions interacting with their genotype. In one embodiment, higher levels of fatty acids and lipids can be obtained under darkness with supplemental organic carbon. In some such embodiments Chlorella protothecoides is utilized. In yet another embodiment, the hydrocarbon yields of one or more of the organisms can be modulated by culture under nitrogen deplete rather than replete conditions. In yet another embodiment, the hydrocarbon composition and yields can be altered by pH or carbon dioxide levels, as is known in the art for Dunaliella.

A variety of different methods are known for the introduction of nucleic acid into host cell chloroplasts and cyanobacteria and any method know in the art may be utilized. Several specific transformation procedures that may be used are detailed in various examples below. In various embodiments, vectors can be introduced into marine algae chloroplasts by, for example without limitation, electroporation, particle inflow gun bombardment, or magnetophoresis.

Magnetophoresis is a nucleic acid introduction technology that also employs nanotechnology fabrication of micro-sized linear magnets (Kuehnle et al., U.S. Pat. No. 6,706,394; 2004; Kuehnle et al., U.S. Pat. No. 5,516,670; 1996, incorporated by reference herein). This technology as described in the prior art and in the new form described herein can be applied to saltwater microalgae and other organisms and thus can be used in the disclosed methods.

In some embodiments a converging magnetic field is used for moving pole magnetophoresis. By using moving magnetic poles to create non-stationary magnetic field lines, as described, plastid transformation efficiency can be increased, in some embodiments, by two orders of magnitude over the state-of the-art of biolistics. Briefly, a magnetophoresis reaction mixture is prepared comprising linear magnetizable particles. The linear magnetizable particles may be comprised of 100 nm tips. They may be, for example, tapered or serpentine in configuration. The particles may be of any combination of lengths such as, but not limited to 10, 25, 50, 100, or 500 um. In some embodiments they comprise a nickel-cobalt core. They may also comprise an optional glass-coated surface.

The magnetizable particles are suspended in growth medium, for example in microcentrifuge tubes. Cells to be transformed are added and may be concentrated by centrifugation to reach a desirable cell density. In some embodiments a cell density of about of 2-4×10̂8 cells/mL is used. Carrier DNA, such as salmon sperm DNA is added, along with linearized transforming vector. In some embodiments about 8 to 20 ug of transforming vector are used, but the amounts of carrier DNA and transforming vector can be determined by the skilled artisan based on the particular circumstances. Finally polyethylene glycol (PEG) is added immediately before treatment and mixed by inversion. In some embodiments filter-sterilized PEG is utilized. For a total reaction volume of 690 uL, approximately 75 uL of a 42% solution of 8000 mw PEG is utilized.

The magnetizable particles are then caused to move such that they penetrate the cells and deliver the transforming vector. In some embodiments the reaction mixture is positioned centrally and in direct contact on a magnetic stirrer, such as a Corning Stirrer/Hot Plate set at full stir speed (setting 10). The stirrer may be heated to between about 39° to 42° C.), preferably to about 42° C. A magnet, such as a neodymium cylindrical magnet (2-inch×¼-inch), is suspended above the reaction mixture, for example by a clamp stand, to maintain dispersal of the nanomagnets. The reaction mixture is stirred for a period of time from about 1 to about 60 minutes or longer, more preferably about 1 to about 10 minutes, more preferably about 2.5 minutes. The optimum stir time can be determined by routine optimization depending on the particular circumstances, such as reaction volume. After treatment the mixture may be transferred to a sterile container, such as a 15 mL centrifuge tube. Cells may be plated and transformants selected using standard procedures.

Polyethylene glycol treatment of protoplasts is another technique that can be used for transformation (Maliga, P. Annu. Rev. Plant Biol. 55:294; 2004).

In various embodiments, vectors can be introduced into Cyanobacteria by conjugation with another prokaryote or by direct uptake of DNA, as described herein and as known in the art.

In various embodiments, the transformation methods can be coupled with one or more methods for visualization or quantification of nucleic acid introduction to one or more algae. Quantification of introduced and endogenous nucleic acid copy number and expression of nucleic acids in transformed cell lines can be performed by Real Time PCR. Further, it is taught that this can be coupled with identification of any line showing a statistical difference in, for example, growth, fluorescence, carbon metabolism, isoprenoid flux, or fatty acid content from the unaltered phenotype. The transformation methods can also be coupled with visualization or quantification of a product resulting from expression of the introduced nucleic acid.

### Genes for Expression

A wide variety of genes can be introduced into the vectors described above for transformation and/or targeted integration into and expression by the chloroplast genome of marine algae or the orthologous gene group of cyanobacteria.

In some embodiments, more than one gene can be introduced into a single vector for coexpression since polycistronic operons are functional in the host cells. For example, two or more genes can be inserted utilizing a multi-cloning site, such as described in Example 22 for a cyanobacteria vector. Two or more genes may also be inserted into an expression vector using unique restriction sites present between coding sequences, for example between the psbB gene and CAT genes in the Dunaliella vectors described below. In other embodiments, two or more genes are introduced into an organism using separate vectors.

In some embodiments, genes that encode a selectable marker are utilized. Selection based on expression of the selectable marker can be used to identify positive transformants. Genes encoding electable markers are well known in the art and include, for example, genes that participate in antibiotic resistance. One such example is the aph(3″)-Ia gene (GI: 159885342) from Salmonella enterica.

Other illustrative genes include genes that participate in carbon metabolism, such as in isoprenoid and fatty acid biosynthesis. In some embodiments, the genes include, without limitation: beta ketoacyl ACP synthase (KAS); isopentenyl pyrophosphate isomerase (IPPI); acetyl-coA carboxylase, specifically one or more of its heteromeric subunits: biotin carboxylase (BC), biotin carboxyl carrier protein (BCCP), α-carboxyltransferase (α-CT), β-carboxyltransferase (β-CT), acyl-ACP thioesterase; FatB genes such as, for example, Arabidopsis thaliana FATB NM_{—}100724; California Bay Tree thioesterase M94159; Cuphea hookeriana 8:0- and 10:0-ACP specific thioesterase (FatB2) U39834; Cinnamomum camphora acyl-ACP thioesterase U31813; Diploknema butyracea chloroplast palmitoyl/oleoyl specific acyl-acyl carrier protein thioesterase (FatB) AY835984; Madhuca longifolia chloroplast stearoyl/oleoyl specific acyl-acyl carrier protein thioesterase precursor (FatB) AY835985; Populus tomentosa FATB DQ321500; and Umbellularia californica Uc FatB2 UCU17097; acetyl-coA synthetase (ACS) such as, for example, Arabidopsis ACS9 gene GI:20805879; Brassica napus ACS gene GI: 12049721; Oryza sativa ACS gene GI:115487538; or Trifolium pratense ACS gene GI:84468274; genes that participate in fatty acid biosynthesis via the pyruvate dehydrogenase complex, including without limitation one or more of the following subunits that comprise the complex: Pyruvate dehydrogenase E1α, Pyruvate dehydrogenase E1β, dihydrolipoamide acetyltransferase, and dihydrolipoamide dehydrogenase; and pyruvate decarboxylase.

Thus, in some embodiments carbon metabolism in a unicellular marine algae or cyanobacteria is modified by integration of one or more of these genes in the host cell plastid genome or orthologous gene group, respectively. In this way, production of a desired hydrocarbon can be obtained, or such production can be increased.

In various embodiments, transformed algae or cyanobacteria may be grown in culture to express the genes of interest. After culturing, the gene products can be collected. For increased biomass production, the algal culture amounts can be scaled up to, for example, between about 1 L to about 10,000 L of culture. Some specific methods for growing transformed algae for expressing genes of interest are described in Example 19 below.

Some embodiments include cultivation of transformed algae and cyanobacteria under heterotrophic or mixotrophic conditions. Use of the novel vectors and transformed algae and cyanobacteria with one or more of the nucleic acids sequences of interest is unique to this invention such that expression of the sequences of interest and their associated phenotypes cannot occur under extended darkness unlike higher plants such as oilseed crops. In addition, such transformed algae can be grown in other culture conditions wherein inorganic nitrogen, salinity levels, or carbon dioxide levels are purposefully varied to alter lipid accumulation and composition.

Thus, in some embodiments an expression vector is prepared comprising a first and second genomic sequence from an organism in which genomic integration and expression of a gene of interest is desired, preferably a unicellular marine algae or a cyanobacteria. The gene or genes of interest are cloned into the vector between the first and second genomic sequences and the organism is transformed with the expression vector. Transformants are selected and grown in culture. The gene product may be collected. However, in some cases a product is collected that is naturally produced by the organism and that is modified, or whose production is modified, by the gene of interest.

The following examples are provided to describe the invention in further detail. These examples serve as illustrations and are not intended to limit the invention. While Dunaliella and Tetraselmis are exemplified, the nucleic acids, nucleic acid vectors and methods described herein can be applied or adapted to other types of Chlorophyte algae, as well as other algae and cyanobacteria, as described in greater detail in the sections and subsequent examples below. While many embodiments and many of the examples refer to DNA, it is understood that particular embodiments are not limited to DNA, and that any suitable nucleic acid can be used where DNA is specified.

### EXAMPLE 1

This example illustrates one possible method for cloning and sequencing of the Dunaliella chloroplast genome.

In this example, Dunaliella is grown in inorganic rich growth medium containing 1 M NaCl at room temperature (20-25° C.). Four liters of culture is grown under illumination with white fluorescent light (80 umol/m²sec) with a 12 hour light: 12 hour dark photoperiod. Algal cells are collected in the late logarithmic phase of growth by centrifugation at 1000×g for 5 min in 500 mL conical Corning centrifuge bottles. The cell pellet is washed twice with fresh growth medium to remove cell surface materials that cause clumping of cells.

The cell pellet is resuspended in ice-cold isolation medium (330 mM sorbitol, 50 mM HEPES, 3 mM NaCl, 4 mM MgCl₂, 1 mM MnCl₂, 2 mM EDTA, 2 mM DTT, 1 mL/L proteinase inhibitor cocktail) to a concentration equivalent to 1 mg chlorophyll per mL of isolation medium. The chlorophyll concentration is estimated by adding 10 uL of the chloroplast suspension to 1 mL of an 80% acetone solution and mixing well. The solution is centrifuged for 2 min at 3000×g. The absorbance of the supernatant is measured at 652 μm using the 80% acetone solution as the reference blank. The absorbance is multiplied by the dilution factor (100) and divided by the extinction coefficient of 36 to determine the mg of chlorophyll per mL of the chloroplast suspension. The solution is adjusted to a concentration of 1 mg chlorophyll per mL with additional cold isolation medium.

The resultant cell suspension in the isolation medium is placed for 2 min in an ice-cold French press at approximately 700 pounds per square inch (psi). The outlet valve is then opened to a flow rate of about 2 mLs/min, and the pressate is collected in a chilled tube containing an equal volume of ice-cold isolation medium. The intact chloroplasts from the pressate are collected as a loose pellet by centrifugation at 1000×g for 5 minutes. The pellet is gently resuspended in 5 mL of cold isolation medium.

For other species, the pressure of the cold French press is set at a pressure determined to be ideal for that species, ranging from 300 psi to 5000 psi. For example, Tetraselmis may be used with a pressure of 3000 to 5000 psi.

After a subsequent washing step, centrifuging as above, the chloroplasts are resuspended in 3 mL of isolation medium per liter of starter culture and loaded on the top of a 30 mL discontinuous gradient of 20, 45, and 65% Percoll in 330 mM sorbitol and 25 mM HEPES-KOH (pH 7.5). Density centrifugation is carried out in a swinging bucket rotor with slow acceleration at 1000×g for 10 mins, then at 4000×g for another 10 min, and then slow deceleration. The intact chloroplasts in the 20-45% Percoll interphase are collected with a plastic pipette. To remove the Percoll, the chloroplast suspension is diluted 10-fold with isolation medium and the chloroplasts are pelleted by centrifugation 1000×g for 2 min. This washing step is repeated once. Washed chloroplasts are then resuspended in a small volume of isolation medium to a chlorophyll concentration of approximately 1 mg/mL.

Plastids are lysed by the addition of an equal volume of lysis buffer containing 50 mM Tris (pH 8), 100 mM EDTA, 50 mM NaCl, 0.5% (w/v) SDS, 0.7% (w/v) N-lauroyl-sarcosine, 200 ug/mL proteinase K, 100 ug/mL RNAse. The solution is mixed by inversion and incubated for 12 hours at 25° C. Lysis of the plastids is confirmed by microscopic examination.

The solution containing plastid DNA is transferred to a polypropylene test tube and ultrapure CsCl is added to a concentration of 1 g/mL. The solution centrifuged at 27,000×g at 20° C. for 30 min in a SW41 swing-out rotor using Beckman #331372 ultracentrifuge tubes. The cleared lysate is collected and transferred to a polypropylene test tube, diluted with sterile deionized distilled water to 0.7-0.8 g/mL CsCl and transferred to 50 mL polyallomer ultracentrifuge tubes (Beckman #3362183). Hoechst 33258 DNA-binding fluorescent dye (0.2 mL of 10 mg/mL) is added to obtain a final concentration of 40 ug/mL in the filled 50 mL ultracentrifuge tube. The tube is filled to maximum with additional 0.8 g/mL CsCl in TE buffer or deionized distilled water, (mass 1.60 to 1.69 g/mL). The sample is centrifuged at 190,000×g (44,300 rpm) at 20° C. for 48 hours in a VTi50 fixed-angle rotor.

Chloroplast DNA is visualized in the resulting gradient using a long-wave UV lamp and the DNA is removed from the gradient with an 18-gauge needle and syringe. The Hoechst 33258 is removed by repeated extractions with 2-propanol saturated with 3 M NaCl and the UV lamp is used to verify complete removal of the dye. The CsCl concentration is reduced by overnight dialysis (Pierce Slide-A-Lyzer 10,000 mwco) against three changes of TE buffer.

DNA is precipitated with 2.5 volumes of 2-propanol plus 0.1 volume of 3 M sodium acetate (pH 5.2) followed by incubation at −20° C. for 1 hour. The solution is transferred to 36 mL centrifuge tubes and spun at 18,000×g, 4° C. for 2 hours. The chloroplast DNA pellet is dried at room temperature and resuspended in 1 mL TE. The solution is extracted three times with phenol-chloroform-isoamyl alcohol (24:24:1) and twice with chloroform-isoamyl alcohol (24:1), mixing by inversion and centrifuging at 1000×g for 10 minutes after each extraction. A second 2-propanol precipitation is performed. The DNA pellet is washed with 70% ethanol, dried, and resuspended in TE buffer. The resulting DNA solution is quantified by optical density at 260 nm.

By this method DNA can be recovered as purified fractions of nuclear, chloroplast and mitochondrial origin. From top to bottom on the cesium chloride gradient, distinct bands of DNA migrate based upon mass, with mitochondrial DNA at top, chloroplast DNA in the middle and nuclear DNA at the bottom of the gradient. Yield of DNA per liter of culture at 2×106 cells/ml are typically 0.9 μg chloroplast DNA and 2.0 μg nuclear DNA.

Shotgun genome sequencing is performed by cloning the chloroplast DNA into pCR4 TOPO blunt shotgun cloning kit according to the manufacturer's instructions (Invitrogen). Shotgun clones are sequenced from both ends using T7 and T3 oligonucleotide primers and a KB basecaller integrated with an ABI 3730XL sequencer (Applied Biosystems, Foster City, Calif.). Sequences are trimmed to remove the vector sequences and low quality sequences, then assembled into contigs using SeqMan II (DNAStar).

Contigs are processed to identify coding regions using the Glimmer program. ORFs (open reading frames) are saved in both nucleotide and amino acid sequence Fasta formats. All putative ORFs are searched against the latest Non-redundant (NR) database from NCBI using the BLASTP program to determine similarity to known protein sequences in the database. A BLAST query of an initial 111 contigs of Dunaliella yielded 273 open reading frames (ORFs), 99 of which have sequence matches that identified a plurality of known as well as chloroplast-encoded genes found in taxa of 9 bacteria, 13 algae, 1 lower plant, 2 higher plants, and 3 others. Results show that the high-molecular weight DNA isolated by this method and used in cloning is indeed the chloroplast genome, based on the matches of the identified proteins with those of other known algae chloroplast-encoded proteins.

### EXAMPLE 2

This example illustrates one possible method for cloning and sequencing of the Tetraselmis spp. chloroplast genome.

Host sequences are preferred for construction of transformation vectors for Tetraselmis spp. Cells are cultured, chloroplasts isolated and lysed, and nucleic acids purified. These consecutive steps are non-obvious for this walled unicellular algae that is recalcitrant to disruption by most organic solvents and robust to high pressure and for which isolated chloroplast DNA has not been reported. Thus, a novel series of steps had to be discovered. The chloroplast isolation method for Tetraselmis adapts certain early elements from a protocol used for isolation of the chloroplast envelope from the wall-less Dunaliella tertiolecta in a clade distinct from Tetraselmis (Goyal et al., Canadian Journal of Botany 76: 1146-1152; 1998, which is incorporated herein by reference in its entirety). The chloroplast lysis and purification of plastid DNA method for Tetraselmis adapts certain elements from a protocol used for the purification of plastid DNA from an enriched rhodoplast fraction of the red macroalga, Gracilaria (Hagopian et al., Plant Molecular Biology Reporter 20: 399-406; 2002, which is incorporated herein by reference in its entirety). Microscopic observations or electrophoretic analyses accompany each step and its optimized modifications for applicability to Tetraselmis.

Tetraselmis spp is grown in 1 L growth medium at room temperature (20°-25° C.) as is known in the art. A ten liter batch culture is grown in a 20 L carboy illuminated with cool and warm white fluorescent light (40-60 umol/m2/s) with a 24 hour light: 0 hour dark cycle. After 12 days cell density is 2.78×10⁶ cells/mL and cells are harvested by centrifugation at 1500×g for 5 mins in 500 mL conical Corning centrifuge bottles. After concentration by centrifugation, the cell pellet is washed once with fresh isolation medium (330 mM sorbitol, 50 mM HEPES, 3 mM NaCl, 4 mM MgCl₂, 1 mM MnCl₂, 2 mM EDTA, 2 mM DTT, 1 ug protease inhibitor cocktail/mL).

The cell pellet is resuspended in 50 mL ice-cold isolation medium (330 mM sorbitol, 50 mM HEPES, 3 mM NaCl, 4 mM MgCl₂, 1 mM MnCl₂, 2 mM EDTA, 2 mM DTT, 1 ug leupeptin/mL). The chlorophyll concentration is estimated by adding 10 ul of the chloroplast suspension to 1 mL of an 80% acetone solution and mixing well. The absorbance of the solution is measured at 652 nm using the 80% acetone solution as the reference blank. The absorbance is multiplied by the dilution factor (100) and divided by the extinction coefficient of 36 to obtain the mg of chlorophyll per mL of the chloroplast suspension. (0.793×100/36=2.2 mg chl/mL). To achieve a concentration equivalent to 1 mg Chl/mL, the 50 mL sample is diluted to 100 mL with additional cold isolation medium.

The resultant 100 mL cell suspension in the isolation medium (final volume is 10 mL per liter of culture before harvest) is placed in an ice-cold French press at 3000 p.s.i. (gauge reading of 1000) in 40 mL aliquots. The outlet valve is then opened to a flow rate of about 2 mL/second, and the pressate is collected in a polypropylene test tube containing an equal volume ice-cold isolation medium. Resulting volume is now 200 mL. The crude chloroplasts from the pressate are collected by centrifugation (1000×g, 3000 rpm in SS34 rotor for 5 minutes) as a three-layer pellet. Approximately 220 mL of dark green translucent supernatant is discarded. The pellet is examined microscopically and determined to contain (from bottom upward) intact cells, phosphate crystals from L1 medium, free chloroplasts. The upper layer is gently resuspended in 30 mL of cold isolation medium. The cell pellet from this suspension is collected in 3 mL of isolation medium and stored overnight at 4° C.

After a subsequent washing step with isolation medium, centrifuging as above, the chloroplast layer is resuspended in 3 mL of isolation medium per liter culture before harvest (33 mL TV). 3 mL of the resulting suspension is loaded on the top of each of 10 discontinuous gradients of 20%, 45%, and 65% Percoll in 330 mM sorbitol, 25 mM HEPES-KOH (pH 7.5). Density centrifugation is carried out at 4° C. in a swinging bucket rotor with slow acceleration to 1000×g and holding for 10 mins, then accelerating to 4000×g for another 10 min, and then slow deceleration (accel and decel setting #5 for the Beckman Allegra centrifuge). The intact chloroplasts in the 45-20% Percoll interface are removed with a polypropylene transfer pipette. To remove the Percoll, the chloroplast suspension is diluted equally with isolation medium and the chloroplasts are pelleted by centrifugation (1000×g; 2 min.). This washing step is repeated once. Washed chloroplasts are then stored overnight at 4° C. The residual Percoll gradients are retained similarly.

On the following day, the chloroplast layer and Percoll gradient cell pellet layers are examined microscopically. The upper layer of the Percoll gradients is also examined and determined to contain mostly free chloroplasts; this material is collected with a polypropylene transfer pipette and washed with an equal volume of isolation medium. Chlorophyll concentration is determined for all three samples and adjusted as necessary to approximately 1 mg/mL. Examples of concentrations and adjustments are as follows: a) 20-45% interface 0.354×100/36=0.98 mg Chl/mL; no adjustment needed; b) Upper Percoll layer=0.273×100/36=0.78 mg Chl/mL; no adjustment needed; and c) Cell pellet=2.2×200/35=12.2 mg Chl/mL; dilute 1:12 with isolation medium. Examples of sample volumes before addition of lysis buffer are as follows: a) 20-45% interface, 4.4 mL; b) Upper Percoll layer, 3.3 mL; and c) cell layer, 12.2 mL.

Plastids are lysed with the addition of an equal volume of lysis buffer: 50 mM Tris (pH 8), 100 mM EDTA, 50 mM NaCl, 0.5% (w/v) SDS, 0.7% (w/v) N-lauroyl-sarcosine (Sigma), 200 ug/mL proteinase K, 100 ug/mL Rnase. Rnase and proteinase K are freshly added from stocks. The solution is mixed by inversion and incubated for 12 hours at 25° C. Lysis of the plastids is determined by microscopic examination of the sample. Both the 20-45% sample and the cell pellet sample contain a translucent supernatant and a dark green, viscous sediment. Microscopy determines that the former is likely to be fully lysed chloroplast material and the latter contains mostly intact algae cells with degraded contents; the cell walls of the algae do not lyse in the presence of detergent and proteinase K.

The samples are allowed to sediment at 4° C. for 3 hours and then the translucent supernatant is carefully aspirated from the viscous dark green material and transferred to a clean polypropylene tube. Supernatant volumes can be as follows: upper Percoll layer 4.3 mL; 20-45% interface 7.6 mL; cell fraction 20 mL. To the supernatant, ultrapure cesium chloride (CsCl, Fluka #20966) is added to a final concentration of 1 g/mL (4.3 g; 7.6 g; 20 g). The solution can then be stored at 4° C. for 48 hours before ultracentrifugation. The solution is then transferred to Beckman #331372 polyallomer 14 mL ultracentrifuge tubes and spun at 27,000×g (12,500 rpm) at 20° C. for 30 min in a SW41 swing-out rotor.

The cleared lysate is collected by attaching an 18 gauge needle to a 10 mL syringe and aspirating the lysate from the base of the centrifuge tube, thus avoiding contamination with the oily fraction at the surface. This lysate is transferred to a clean polypropylene test tube, diluted with sterile ddH₂0 water to 0.7-0.8 g/mL CsCl and transferred to Beckman Optiseal #362183 polyallomer 36 mL ultracentrifuge tubes. Hoechst 33258 (0.2 mL of 10 mg/mL) is added to a final concentration of 50 ug/mL and the tubes are filled to maximum with additional 0.7 g/mL CsCl. The samples are centrifuged at 190,000×g (44,300 rpm) at 20° C. for 48 hours in a VTi50 fixed-angle rotor.

A long-wave UV lamp (365 nm) is used to visualize the chloroplast DNA band above the nuclear DNA band and the DNA is removed from the gradient with a 20-gauge needle and 10 cc syringe. Samples are dispensed from the syringe into a 15 mL polypropylene tube after removal of the needle to avoid unnecessary shearing of the DNA. The samples are stored overnight at 4° C. Hoechst 33258 is removed from the aqueous DNA-containing samples by two extractions with an equal volume of isopropanol saturated with 3 M NaCl (80 mL isopropanol plus 20 mL 3M NaCl) and the UV lamp is used to verify complete removal of the dye. The CsCl concentration is reduced by overnight dialysis (Pierce Slide-A-Lyzer 10,000 molecular weight cutoff) against three changes of TE (10 mM Tris 7.5, 1 mM EDTA 8.0).

DNA is precipitated with 0.1 volumes of 3 M sodium acetate (pH 5.2) plus 2.5 volumes of 2-propanol, mixing, and then incubating at −20° C. overnight. The DNA is pelleted in Oakridge #3119-0050 50 mL centrifuge tubes and spun at 18,000×g, 4° C. for 1 hour (12,300 rpm on RC6 centrifuge with SS-34 rotor). The chloroplast DNA pellets are dried at room temperature and resuspended in 1 mL TE. The solution is then extracted three times with phenol-chloroform-isoamyl alcohol (24:24:1) and twice with chloroform-isoamyl:alcohol (24:1), mixing by inversion. A second 2-propanol precipitation is performed, pellets are washed with 70% ethanol, dried, and resuspended in TE.

By this method DNA can be recovered as purified fractions of nuclear, chloroplast and mitochondrial origin. From top to bottom on the cesium chloride gradient, distinct bands of DNA migrate based upon mass, with mitochondrial DNA at top, chloroplast DNA in the middle and nuclear DNA at the bottom of the gradient. Yield of DNA per liter of culture at 2×10⁶ cells/ml are typically 0.8 μg chloroplast DNA and 2.5 μg nuclear DNA.

The nucleic acid samples are then used for shotgun genome sequencing and analyses as described in Example 1.

### EXAMPLE 3

This example illustrates one possible method for preparation of backbone vectors for targeted integration of DNA segments in the chloroplast genome.

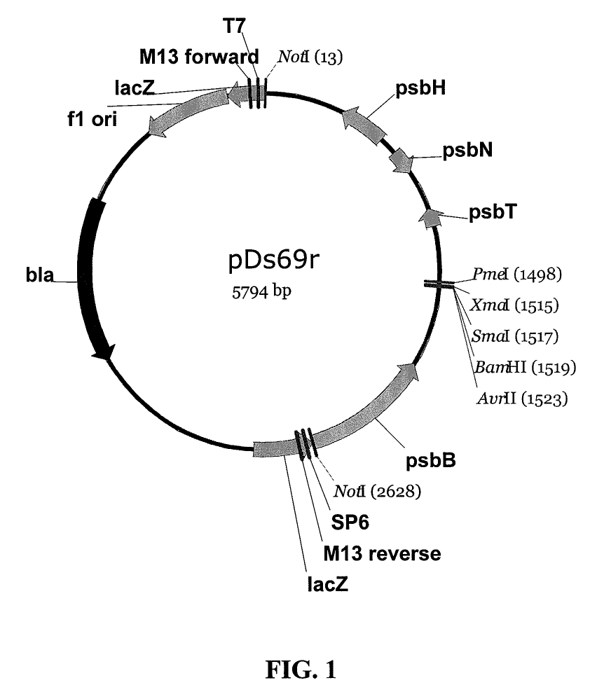

Backbone vectors are desired for targeted integration of DNA segments in the chloroplast genome. In one embodiment of this example, chloroplast DNA sequences derived from sequencing the genome of Dunaliella spp are used to produce chloroplast transformation vector pDs69r (FIG. 1). PCR primer 5′caggtttgcggccgcaagaaattcaaaaacgagtagc3′ (SEQ ID NO: 83) and 5′aagacccgggatcctaggtcgtatattttcttccgtatttat3′ (SEQ ID NO: 84) are used to amplify a fragment of Dunaliella salina chloroplast DNA including the psbH, psbN, and psbT genes and adding a NotI restriction site (5′CCATGG3′) to one end of the DNA molecule and restriction sites for AvrII (CCTAGG), BamHI (GGATCC), SmaI (CCCGGG) to the other end. Amplification is performed with a Pfx proof reading enzyme (Accuprime Pfx, Invitrogen, Carlsbad, Calif.) from a chloroplast DNA preparation of Dunaliella salina using the following conditions; 95° C. 5 min, (94° C. 45 sec, 55° C. 60 sec, 68° C. 90 sec) for 25 cycles, 68° C. 7 min. A second DNA product is amplified with primers 5′aatttttttttataaatacggaagaaaatatacgagctaaattttatgttcttccgtt3′ (SEQ ID NO: 1) and 5′tatggggcggccgcctttattataacataatgaatg3′ (SEQ ID NO: 2) using the same parameters to produce a molecule containing the psbB gene and placing a NotI restriction site on one end of the molecule. The two PCR products are digested with BamHI and ligated together, followed by digestion with NotI. The resulting product is cloned into the NotI site of the multipurpose cloning vector pGEM13Z (Promega). This vector is named “pDs69r”. Using this general strategy, additional Dunaliella and Tetraselmis vectors may be generated based on the sequence database obtained from Examples 1 or 2.

Following is the sequence of the pGEM13Z vector backbone into which chloroplast vector sequences are cloned. NotI (position 2628) through NotI (position 13) of pDS69r:

(SEQ ID NO: 3)

5′ggccgctccctggccgacttggcccaagcttgagtattctatagtgtc
acctaaatagcttggcgtaatcatggtcatagctgtttcctgtgtgaaat
tgttatccgctcacaattccacacaacatacgagccggaagcataaagtg
taaagcctggggtgcctaatgagtgagctaactcacattaattgcgttgc
gctcactgcccgctttccagtcgggaaacctgtcgtgccagctgcattaa
tgaatcggccaacgcgcggggagaggcggtttgcgtattgggcgctcttc
cgcttcctcgctcactgactcgctgcgctcggtcgttcggctgcggcgag
cggtatcagctcactcaaaggcggtaatacggttatccacagaatcaggg
gataacgcaggaaagaacatgtgagcaaaaggccagcaaaaggccaggaa
ccgtaaaaaggccgcgttgctggcgtttttccataggctccgcccccctg
acgagcatcacaaaaatcgacgctcaagtcagaggtggcgaaacccgaca
ggactataaagataccaggcgtttccccctggaagctccctcgtgcgctc
tcctgttccgaccctgccgcttaccggatacctgtccgcctttctccctt
cgggaagcgtggcgctttctcatagctcacgctgtaggtatctcagttcg
gtgtaggtcgttcgctccaagctgggctgtgtgcacgaaccccccgttca
gcccgaccgctgcgccttatccggtaactatcgtcttgagtccaacccgg
taagacacgacttatcgccactggcagcagccactggtaacaggattagc
agagcgaggtatgtaggcggtgctacagagttcttgaagtggtggcctaa
ctacggctacactagaagaacagtatttggtatctgcgctctgctgaagc
cagttaccttcggaaaaagagttggtagctcttgatccggcaaacaaacc
accgctggtagcggtggtttttttgtttgcaagcagcagattacgcgcag
aaaaaaaggatctcaagaagatcctttgatcttttctacggggtctgacg
ctcagtggaacgaaaactcacgttaagggattttggtcatgagattatca
aaaaggatcttcacctagatccttttaaattaaaaatgaagttttaaatc
aatctaaagtatatatgagtaaacttggtctgacagttaccaatgcttaa
tcagtgaggcacctatctcagcgatctgtctatttcgttcatccatagtt
gcctgactccccgtcgtgtagataactacgatacgggagggcttaccatc
tggccccagtgctgcaatgataccgcgagacccacgctcaccggctccag
atttatcagcaataaaccagccagccggaagggccgagcgcagaagtggt
cctgcaactttatccgcctccatccagtctattaattgttgccgggaagc
tagagtaagtagttcgccagttaatagtttgcgcaacgttgttgccattg
ctacaggcatcgtggtgtcacgctcgtcgtttggtatggcttcattcagc
tccggttcccaacgatcaaggcgagttacatgatcccccatgttgtgcaa
aaaagcggttagctccttcggtcctccgatcgttgtcagaagtaagttgg
ccgcagtgttatcactcatggttatggcagcactgcataattctcttact
gtcatgccatccgtaagatgcttttctgtgactggtgagtactcaaccaa
gtcattctgagaatagtgtatgcggcgaccgagttgctcttgcccggcgt
caatacgggataataccgcgccacatagcagaactttaaaagtgctcatc
attggaaaacgttcttcggggcgaaaactctcaaggatcttaccgctgtt
gagatccagttcgatgtaacccactcgtgcacccaactgatcttcagcat
cttttactttcaccagcgtttctgggtgagcaaaaacaggaaggcaaaat
gccgcaaaaaagggaataagggcgacacggaaatgttgaatactcatact
cttcctttttcaatattattgaagcatttatcagggttattgtctcatga
gcggatacatatttgaatgtatttagaaaaataaacaaataggggttccg
cgcacatttccccgaaaagtgccacctgacgtctaagaaaccattattat
catgacattaacctataaaaataggcgtatcacgaggccctttcgtctcg
cgcgtttcggtgatgacggtgaaaacctctgacacatgcagctcccggag
acggtcacagcttgtctgtaagcggatgccgggagcagacaagcccgtca
gggcgcgtcagcgggtgttggcgggtgtcggggctggcttaactatgcgg
catcagagcagattgtactgagagtgcaccatatgcggtgtgaaataccg
cacagatgcgtaaggagaaaataccgcatcaggaaattgtaagcgttaat
attttgttaaaattcgcgttaaatttttgttaaatcagctcattttttaa
ccaataggccgaaatcggcaaaatcccttataaatcaaaagaatagaccg
agatagggttgagtgttgttccagtttggaacaagagtccactattaaag
aacgtggactccaacgtcaaagggcgaaaaaccgtctatcagggcgatgg
cccactacgtgaaccatcaccctaatcaagttttttggggtcgaggtgcc
gtaaagcactaaatcggaaccctaaagggagcccccgatttagagcttga
cggggaaagccggcgaacgtggcgagaaaggaagggaagaaagcgaaagg
agcgggcgctagggcgctggcaagtgtagcggtcacgctgcgcgtaacca
ccacacccgccgcgcttaatgcgccgctacagggcgcgtccattcgccat
tcaggctgcgcaactgttgggaagggcgatcggtgcgggcctcttcgcta
ttacgccagctggcgaaagggggatgtgctgcaaggcgattaagttgggt
aacgccagggttttcccagtcacgacgttgtaaaacgacggccagtgaat
tgtaatacgactcactatagggcgaattggc3′

Following is the sequence of the pDS69r Dunaliella salina chloroplast DNA fragment from NotI (position 13) through NotI (position 2628). This segment was cloned as two fragments and ligated together:

(SEQ ID NO: 4)

5′ggccgcctttattataacataatgaatgactaatgtcaattgtttatt
tgaaaattaacttcaataaaaatttacaaagagaaaaaaattaaccggat
ttttctttgataaaaatacgtaggaaacaatattttattttgtttataac
aaaaaaaagtttaaaatgaaaaaatcacgtttataccgaatttaaacgtt
tactattaatactaatgaatttaatgtactaataagaagagttatataac
tattcaaattaacaaaaagttaaaaggaaacctcctgtgttttaattaaa
acacaggaggtttatctcatttacttgataacaaaatattaaagaagtga
tatttctatctgggtttcaaacgcaagggcctcttagagaggaacacttt
aaattatataaatttatttagcggctaaactttcccagctattagtaaca
ccatctaaaattaatgaactattataaatttctagaataataagtaaaaa
aaccgcaaataaaagaattgctacagccataagaactgtagtaccccatc
caggtaaaactttacctgcttcagagtttagaggacgtaataaagttcct
aatggtgtaacaattcctggttcttgtgatgttgaagtttgtgtactatt
ttttcctgtagccataattgatagttaataaaatctttttgtttttttcc
tttctgtaatattgtataatatatatggagaataattttgtcttgtcaaa
aattttaaatttatggaaagtccggcttttttctttaccttctttttatg
gtttcttttattaagtgctacaggttattcagtttatgttagttttggac
ctccttcaagaaaattgagagatccttttgaagaacatgaagattaaatt
aataatcttagttaagtaaaaattttaagtattctaagggttggacttca
ctaattaatgttaatgaaatccaacccttataatacttcatttgaaacgt
atttacgataaatatagaatttctcgtagattttcgtatcggaaaaaaca
actttattgtttggtccgacaagtaattttaataaaaaattattctatta
ctattttgcaatacgtggaggctctctaaaaaagatagagaaaaagataa
tacctaacgttccaattaataagaaagtgtaaactaaagcttccatgaaa
ggtgtttaataaatttattgaaaagactagtcttttcaaataggaacata
ataccaaattttacattagtgtaaaacaaaaagaattttcttccgaatta
cgaaaagaaaataaacgaagcggtcagaagataaatttaaaatatctaac
gacttacctaaagttataaaagataaaatttaattccaataaggagttaa
aaaaaatattatcttagatttttttaacaaaaataaaatattaacatttt
ataaaaataaaacggaagaacataaaatttagcgtttaaacgaattcgcc
cttcccgggatcctaggtcgtatattttcttccgtatttataaaaaaaaa
ttctttttatgaaataaactttgatcaaatttgtttacactaactcaaat
tcttttgctcagagaaaatctaagcccatctaaaaaaaaaaaaacaatta
taccgtattaaaatctacggtaagatagaaaatctaataaagataagaaa
aatcacattacaaaaaaatcacattacaaaatatgtgaactttgttaaat
gaatcttctattttctagtcggaaaacaaaaaaacaaagaaaagtgttta
gtccgccaaaaagagaaaaaatctattagaatttctcgacggaaattcta
atagattttttctatatgaatttaaaaacaagaatttctaaatattcttg
gtagaatattggaataaaacttaatatagtgattagaaagcttcacgaac
agatgaagtatcaccaagtttcttatatttaccgaattctaattgatcat
taatgtcttcatcaataccagcgaaaacgtcacggaaaatagttcttgaa
ccatgccaaatatgaccaaagaagaataataaggcaaaagataagtgtcc
aaaagtgaaccaaccacgtgggctactacggaatacaccgtcagattgta
aagtcgaacggtcaaattcaaagatttcacctaattgagctttacgtgca
tattttttaacagttgaagggtcagtaaatgttaaaccatttaattcacc
accatagaatgtaactgaaacaccaacttgttcaattgagtattttgatt
cagctttacggaatggtacgtcagcacgaacaacaccgtctttatcaatt
aaaacaacagggaaagtttcaaagaaagtaggcatacgacgaacaaaaag
ttcacgaccttcttgatctttaaaactagcgtgtcctaaccaacctacag
cgataccatcaccactgttcatagcacctgtacggaataatccaccttta
gctgggttattaccaatgtaatcatagaaagctaatttttcaggaatttt
tgcccaagcttctgaaacagataaaccttcagatgtactttgtgctactc
gtttttgaatttcttgc3′

### EXAMPLE 4

This example illustrates one possible method for introduction of regulatory sequences into vectors for targeted integration of DNA segments in the chloroplast genome.

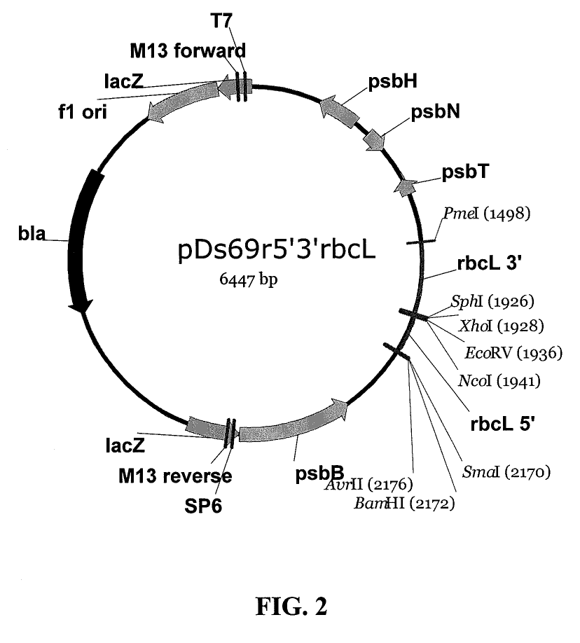

Regulatory sequences are desired in some cases for inclusion in chloroplast vectors. Additional regulatory sequences commonly used in higher plant plastids, but not discussed in detail here include, for example, the psbA promoter, the psbD promoter, the atpB promoter, the atpA promoter, the Prrn promoter, and additional promoter sequences as described in U.S. Pat. No. 6,472,586, which is incorporated herein by reference in its entirety. One possible 3′ UTR sequence which can be used is, for example without limitation, the rbcL 3′ UTR (Barnes et al., (2005) Mol. Gen. Genomics 274:625-636). In a specific exemplified embodiment, nucleic acid sequences for regulating expression of genes introduced into the chloroplast genome by vector pDs69r are introduced by PCR cloning of the Dunaliella rbcL 5′ and 3′ UTR to produce pDs69r5′3′rbcL (FIG. 2). Using the PCR cycling conditions listed in Example 3, primers

(SEQ ID NO: 5)

5′TATTAATCCTAGGATCCCGGGTTATATATAGTTAATTTTTATAAAA
G3′
and

(SEQ ID NO: 6)

5′TAAACCCGTTTAAACTTGCATGCCTCGAGGATATCACCATGGTATTAT
CTAAAAATGAAACAT3′

are used to amplify Dunaliella salina rbcL5′ UTR, placing recognition sequence for the restriction enzymes AvrII (CCTAGG), BamHI (GGATCC) and SmaI (CCCGGG) on the 5′ end, and recognition sequence for the restriction enzymes NcoI (CCATGG), EcoRV (GATATC), XhoI (CTCGAG), SphI (GCATGC), and PmeI (GTTTAAAC) on the 3′ end of the molecule. The PCR product is digested with AvrII and XhoI. A second PCR product amplifying the rbcL 3′ UTR is produced using primers ′TGATATCCTCGAGGCATGCTTTTTTCTTTTAGGCGGGTCCGAAG3′ (SEQ ID NO: 7) and 5′TTCGTCTAGTTTAAACTTAGCGCAGCGGACAGACAAC3′ (SEQ ID NO: 8), and recognition sequence for the restriction enzymes XhoI (CTCGAG), SphI (GCATGC) are added to the 5′ end of the molecule and PmeI (GTTTAAAC) is added to the 3′ end of the molecule. The PCR product is digested with XhoI and PmeI. The 248 bp rbcL5′ UTR and 430 bp rbcL3′ UTR restriction-digested PCR products are then simultaneously cloned into the AvrII and PmeI sites of pDs69r. The resulting molecule is “pDs69r5′3′rbcL”. This general strategy can be employed to produce additional Dunaliella and Tetraselmis vectors based on the sequence database obtained from Examples 1 and 2.

Following is the sequence of the pDs69r5′3′rbcL Dunaliella salina chloroplast rbcL 5′ UTR PCR product. The sequence includes from the AvrII restriction site (position 2176) through the XhoI site (position 1928), in the sense orientation of the promoter/5′ UTR:

(SEQ ID NO: 9)

AvrII-gatcccgggttatatatagttaatttttataaaagaaaattaaa
caaataaagcataataagttattataaatacaggaacgaaattatataga
attataatttataaattggaaattagaaaaaaattatatgttctttaatt
accaaaatttaaatttggtaaaagattattatatcatcggatagattatt
ttaggatcgacaaaaatgtttcatttttagataataccatggtgatatcc
tcga-XhoI

Following is the sequence of the pDs69r5′3′rbcL Dunaliella salina chloroplast rbcL 3′ UTR PCR product. The sequence includes from the XhoI site (position 1928) through PmeI site (position 1498) in the sense orientation of the 3′ UTR:

(SEQ ID NO: 10)

XhoI-ggcatgcttttttcttttaggcgggtccgaagtccttaggcttat
tcgaaggaaaaacgagaaaaatttacgtagtaaattttctttgctggccc
tgccaaaaacaacaccattaacctataagtagtaataattctttagtatt
acttttaggttatttataaatttgagaagtatagaagaatctatagattt
tgcttatgtgtttatctatagattcttctatacttctcatttttaacaaa
tttttattaagatttttttaaacaaaaaaaaagttttcaacttatataat
taaacctaaacaacgttgtatattttttattttaagttttggtaaagtat
gtataccagtaaacctttagtaaatttttttaccgcttaggctaggacct
ataaaatttagcgcggcgcaagggcgaattcgttt-PmeI

### EXAMPLE 5

This example illustrates another possible method for introduction of regulatory sequences into vectors for targeted integration of DNA segments in the chloroplast genome.

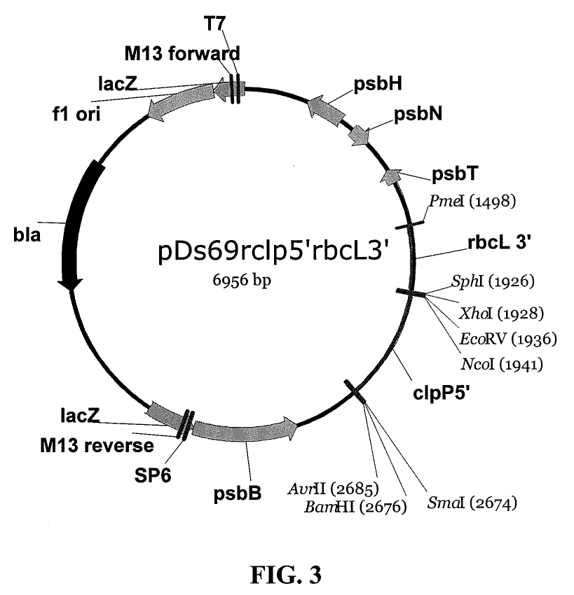

Another specific exemplified embodiment of chloroplast regulatory sequences included in a chloroplast vector is pDS69r5′clpP. The clpP protease promoter can be used to drive expression of transgenes in higher multicellular plants (U.S. Pat. No. 6,624,296). The gene clpP is a natural chloroplast gene in Chlamydomonas algae that can provide a benefit to algae cells grown under conditions of high light and/or high CO₂ (Majeran et al., The Plant Cell 12:137-149; 2000, which is incorporated herein by reference in its entirety). These conditions are now known to be suited to culture of algae in outdoor bioreactors or raceways and using flue gas emissions including carbon dioxide for sequestration by algae (Huntley M E and D G Redalje. Mitigation and Adaptation Strategies for Global Change 12: 573-608; 2007). In turn, these conditions are conducive to biomass and fatty acid production in target algae using the embodied chloroplast-based expression of genes for production of biofuels in algae. Primers 5′ACGTTATTAATCCTAGGATCCCGGGCACTCAAAAGATAGGACGACGA3′ (SEQ ID NO: 11) and 5′GTTTAAACTTGCATGCCTCGAGGATATCACCATGGCCTTTAAGTAGAGGATGC (SEQ ID NO: 12) AT3′ are used with the above cycling conditions to PCR amplify a 785 base pair product containing 683 base pairs of the Dunaliella salina clpP promoter and 5′ UTR sequence. It also includes recognition sequence for the restriction enzymes AvrII (CCTAGG), BamHI (GGATCC) and SmaI (CCCGGG) on the 5′ end, and recognition sequence for the restriction enzymes NcoI (CCATGG), EcoRV (GATATC), XhoI (CTCGAG), SphI (GCATGC), and PmeI (GTTTAAAC) on the 3′ end of the molecule. The PCR product is digested with BamHI and EcoRV and cloned into the BamHI and EcoRV sites of pDs69r5′3′rbcL. The resulting molecule is “pDS69r5′clpP3′rbcL” (FIG. 3). Using this general strategy, additional Dunaliella and Tetraselmis vectors may be generated based on the sequence database obtained from Examples 1 and 2.

Following is the sequence of the clpP protease promoter and 5′UTR sequences for D. salina from genome sequencing project contig #409:

(SEQ ID NO: 13)

CACTCAAAAGATAGGACGACGATTAAGAAAAAACAATATATATATGCCAA
TTGGTGTTCCACGTATTATTTATAGTTGGGGTGAAGAACTTCCAGCTCAA
TGGACTGATATTTATAATTTTATTTTCCGTCGAAGAATGGTTTTTTTAAT
GCAATATTTAGATGACGAACTTTGTAACCAAATTTGTGGTTTATTAATTA
ATATCCATATGGAAGATCGATCTAAAGAACTTGAAAAAAACGAAGTCGAA
GGAGATTCAAAACCTCGTTCAACTAGTAGTGAAAAGAGAACTGATGGTCC
ATCTTCTGTGAAGAAAAATAGATCTCCTGAAGATTTATTAAATGCTGATG
AAGATTTAGGTATTGATGATATTGATACATTAGAACAATTAACATTACAA
AAAATTACAAAAGAATGGCTAAATTGGAATTCACAGTTTTTTGATTATTC
AGATGAACCTTATTTATATTATTTAGCACAAACTTTATCAAAAGATTTTG
GTAATAGCWMTTcTMGtYSGCCttRCGAtWTTMRYSCWcACAAttTTTTa
AtAGtTTAAAAAGTAATTCCttAAACTTACAAAATAGAAAAAGTGCACCT
TCtGGTAAAGGaCTAgATATTTAtTCAGCATTTAGAACAAGTTTAAATTT
TGAAAATGAAGGTGCGGGTGCATATAGCTTAAA

Following is the sequence of the primers for clpP protease promoter with added restriction sites (AvrII, BamHI and SmaI) on 5′ end and PmeI, SphI, XhoI, EcorV, and NcoI on 3′ end: 5′ end 5′acgttattaatcctaggatcccgggcactcaaaagataggacgacga3′ (SEQ ID NO: 14) 3′ end 5′aaacttgcatgcctcgaggatatcaccatggcctttaagtagaggatgcat3′ (SEQ ID NO: 15) Following is the sequence of the PCR product after cleavage with BamHI and EcoRV:

(SEQ ID NO: 16)

gatcccgggcactcaaaagataggacgacgaCACTCAAAAGATAGGACGA
CGATTAAGAAAAAACAATATATATATGCCAATTGGTGTTCCACGTATTAT
TTATAGTTGGGGTGAAGAACTTCCAGCTCAATGGACTGATATTTATAATT
TTATTTTCCGTCGAAGAATGGTTTTTTTAATGCAATATTTAGATGACGAA
CTTTGTAACCAAATTTGTGGTTTATTAATTAATATCCATATGGAAGATCG
ATCTAAAGAACTTGAAAAAAACGAAGTCGAAGGAGATTCAAAACCTCGTT
CAACTAGTAGTGAAAAGAGAACTGATGGTCCATCTTCTGTGAAGAAAAAT
AGATCTCCTGAAGATTTATTAAATGCTGATGAAGATTTAGGTATTGATGA
TATTGATACATTAGAACAATTAACATTACAAAAAATTACAAAAGAATGGC
TAAATTGGAATTCACAGTTTTTTGATTATTCAGATGAACCTTATTTATAT
TATTTAGCACAAACTTTATCAAAAGATTTTGGTAATAGCWMTTcTMGtYS
GCCttRCGAtWTTMRYSCWcACAAttTTTTaAtAGtTTAAAAAGTAATTC
CttAAACTTACAAAATAGAAAAAGTGCACCTTCtGGTAAAGGaCTAgATA
TTTAtTCAGCATTTAGAACAAGTTTAAATTTTGAAAATGAAGGTGCGGGT
GCATATAGCTTAAAatgcatcctctacttaaaggccatggtgat

### EXAMPLE 6

This example illustrates another possible method for introduction of regulatory sequences into vectors for targeted integration of DNA segments in the chloroplast genome.

In another specific example, the chloroplast endogenous regulatory sequences are the promoter and the 5′ untranslated sequences of the psbD gene to produce chloroplast vector pDspsbDCAT.

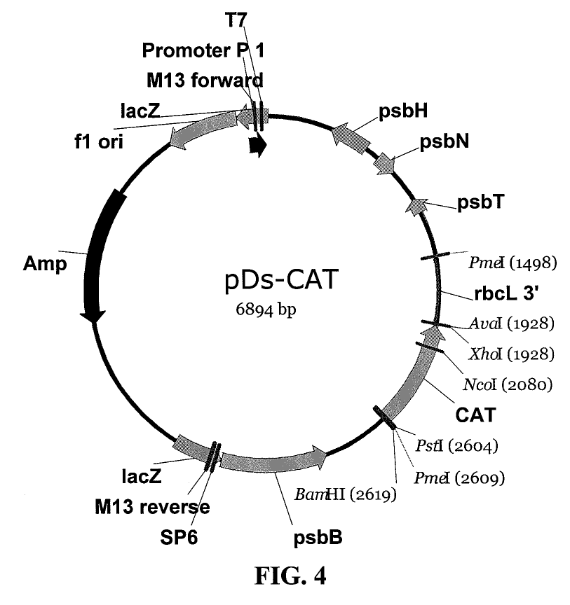

The plasmid pDs69rCAT, as described in the subsequent Example 7, is cleaved by BamHI and XhoI enzymes to release the CAT gene which is subsequently replaced with a BamHI-PstI-CAT-XhoI fragment. The resulting clone is named “pDsCAT” (FIG. 4). To produce “pDsCAT”, primer “psbDCAT-L” 5′atactaggatccgtttaaacctgcagATGgagaaaaaaatcactgg 3′ (SEQ ID NO: 59) and primer “psbDCAT-R” 5′cacgtgggtaccctcgagaagcttTTAcgcc 3′ (SEQ ID NO: 60) are used to amplify the 710 bp BamHI-PstI-CAT-XhoI DNA molecule using pDs69rCAT as a template and using the following conditions; 95° C. 5 min, (94° C. 45 sec, 60° C. 60 sec, 68° C. 90 sec) for 25 cycles, 68° C. 7 min. The resulting DNA fragment is cloned into pCR4TopoBlunt general purpose cloning vector, digested with BamHI and XhoI, gel purified and ligated into the BamHI and XhoI sites of pDs69rCAT.

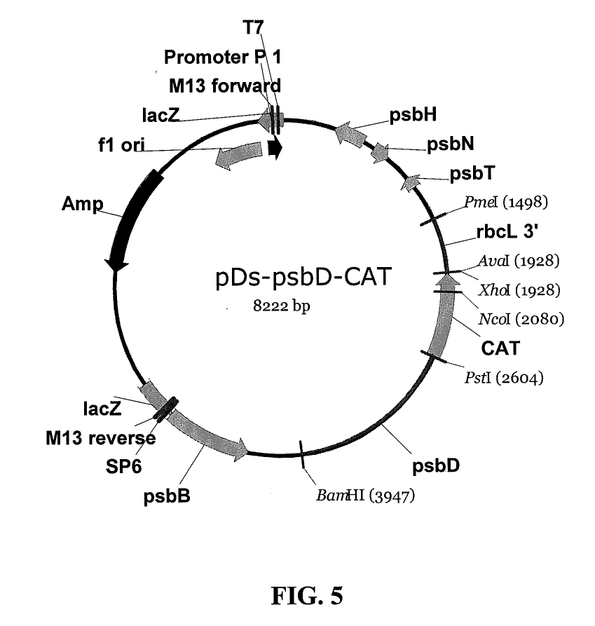

To PCR amplify the Dunaliella salina psbD promoter, primer “psbD-L” 5′CCGCCGGGCGGATCCCTGTAAGTTTCTTTCAAAAATACATG 3′ (SEQ ID NO: 17) and primer “psbD-R” 5′GTCCCGAAGTCCTGCAGTGCGTGCATCTCCATAATAATT 3′ (SEQ ID NO: 18) are used to amplify the 1373 bp product using genomic DNA as a template and the following conditions; 95° C. 5 min, (94° C. 45 sec, 62° C. 60 sec, 68° C. 90 sec) for 25 cycles, 68° C. 7 min. The resulting DNA fragment is cloned into pCR4TopoBlunt general purpose cloning vector. Then, the psbD promoter in pCRTopoBlunt is digested with BamHI and PstI, the 1351 base pair product is gel purified and ligated into the gel-purified linear fragment of pDsCAT digested with BamHI and PstI. The resulting chloroplast vector molecule is “pDspsbDCAT” (FIG. 5). Using this general strategy, additional Dunaliella and Tetraselmis vectors may be generated based on the sequence database obtained from Examples 1 and 2.

Following is the sequence of the pDSCAT PCR product (product size: 710 bp) for cloning into pCR4TopoBlunt vector:

(SEQ ID NO: 19)

5′atactaggatccgtttaaacctgcagATGgagaaaaaaatcactggat
ataccaccgttgatatatcccaatggcatcgtaaagaacattttgaggca
tttcagtcagttgctcaatgtacctataaccagaccgttcagctggatat
tacggcctttttaaagaccgtaaagaaaaataagcacaagttttatccgg
cctttattcacattcttgcccgcctgatgaatgctcatccggaattccgt
atggcaatgaaagacggtgagctggtgatatgggatagtgttcacccttg
ttacaccgttttccatgagcaaactgaaacgttttcatcgctctggagtg
aataccacgacgatttccggcagtttctacacatatattcgcaagatgtg
gcgtgttacggtgaaaacctggcctatttccctaaagggtttattgagaa
tatgtttttcgtctcagccaatccctgggtgagtttcaccagttttgatt
taaacgtggccaatatggacaacttcttcgcccccgttttcaccatgggc
aaatattatacgcaaggcgacaaggtgctgatgccgctggcgattcaggt
tcatcatgccgtttgtgatggcttccatgtcggcagaatgcttaatgaat
tacaacagtactgcgatgagtggcagggcggggcgTAAaagcttctcgag
ggtacccacgtg3′

Following is the sequence of the Dunaliella salina psbD promoter 1373 bp PCR product for cloning into pCR4TpopBlunt vector:

(SEQ ID NO: 20)

5′CCGCCGGGCGGATCCCTGTAAGTTTCTTTCAAAAATACATGTCCATTT
TTTTATAAACAAACGGGAGGGGTCGTCTCATAAAAAGGAAATTTTTCTTA
AACAATTTTAGCGAAGCGGTCAGAGAAAATTATATTAGAATTTCTCGAAG
ATTTTCAATATCTCAAAGAGCAGGACCGATTGAAAACTTCGATATTTTCT
AAAACTCTTTTGACTTTTCGTGAGATAAAATAAAAGAGATACAGTCAATA
ATAAATTTAACTTGATTAAATTTATTCTTTTCCGTTCTTGTTTTTTTCTA
ATTTACAGTATTAAAACAGAAAAAAAGTAAGGCTAAATATCTTAAGGAAA
TATAAAACACAATTGTTTTTTTCAAATTTTTGGTTTTTTGAAAAATTAAA
CAAATAAAAGCAGTAAAACGTAGAAAATATAGAAGTTCTAAATACCAGGA
GATAAACCCTTTGGGTTTATCTTTTTGCTGCACTAATTAAAAAACGATTT
TATAATCATATAGAATCCGATTAAGATAGTTTGATTTGTTATTGTTTCAT
TAATTTTTAATTGATAACTTGCATTAGTTTATAACTATCGGATTTTTCCT
TAAGAAAAATCCGTAGGAAAAAATCTTTTAAAATATTTTTTGTAAGAAAA
ATCAATCTATCAGATTACAATTTTATTTCAAGCCTATCTTTTTATTAATT
CAATTCAAACGAGGATGTTCTCTATTGAGAATTAGGATTCTTTTCAAGAC
TTAATACATATACTTTTACTTATTGTATTATTAATAATAATGGTTTTATT
AAAAAAAATTATAATATCTACTAAACATTTAACATTAGGCGGGTTCGTTA
ACCTTTAAGGTTAAAGAGATATATGTTAAATTAAACATAAACGAAAAGAC
TTTAAATTTTTCAAATAAAAAAAAAGATACAGAGGGTACTAATATTTAAT
ATTATGACCTTCTGTATCCTATACTTAATAAGTATAAATTATAATATAGA
TTAATAAATCTATTCAAGTTAATAAACTGTGTTTTTATTTTATTTAATGA
TTTTCTCTACTAAATATTAAATATGTTATTATTTATACATAGTGTTTTTT
CTTTTTTTTTTTTAAGCCTGTTTAACTCAATCGGTAGAGTATTGGTTTTG
TAAACCAAAGGTTGCGGGTTCGATTCCTGTAGCAGGCTACTAATTTTTTA
AGATATTTTATATTTTAAAAATATCTTTTTAAAATAAAAAAAAAATTTTT
TAAATCGATTTTAAAAATAAAAAAAGCTATACTTATAAATGCAATAAAGG
TTAAAAAAAAAATTAAACGATATGATGAATTATAAAAATTATTATGGAGA
TGCACGCACTGCAGGACTTCGGGAC 3′

### EXAMPLE 7

This example illustrates one possible method for introduction of selectable marker sequences into vectors for targeted integration of DNA segments in the chloroplast genome.

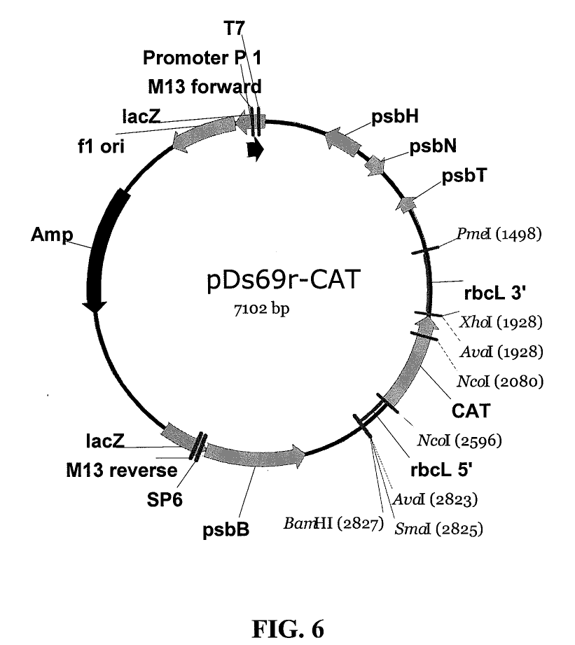

Targeted integration segments can be used, for example, to facilitate selection of transplastomic algae by resistance to antibiotics, such as chloroplast vectors pDs69r-aadA, pDs69r-aphA6, and pDs69r-CAT (FIG. 6) for resistance to spectinomycin, kanamycin, and chloramphenicol along with any relevant analogues.

The aadA gene of Escherichia coli transposon Tn7, encoding the aminoglycoside 3′ adenylyltransferase enzyme ANT(3″)-Ia, is isolated from plasmid p657 (Fargo et al., Mol. Gen. Genet. 257:271-282; 1998, which is incorporated herein by reference in its entirety) by NcoI and SphI digestion. The resulting 807 base pair product is ligated into the NcoI and SphI sites of pDs69r, producing vector pDs69r-aadA.

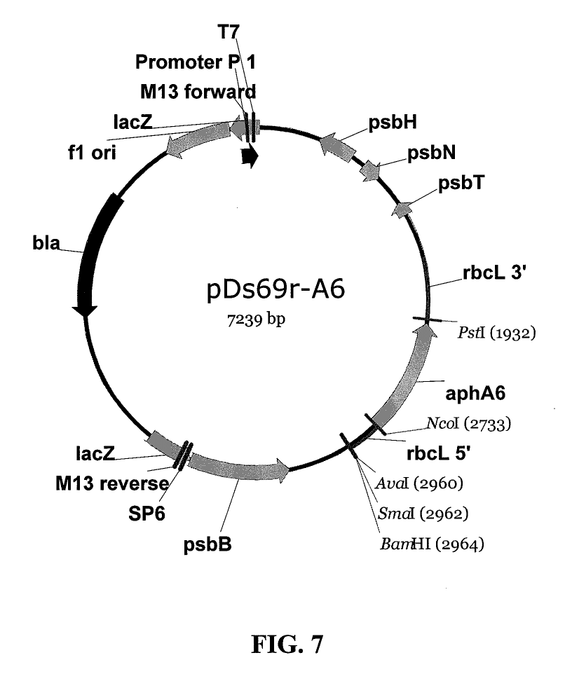

Forward primer 5′CATTTTTAGATAATACCATGGAATTACCAAATATTA3′ (SEQ ID NO: 21) and reverse primer 5′ GCATGCCTGCAGAGTATTTTAGATAATGCTTGGAATCAATTCAATTCATCAAGT TTTAAA3′ (SEQ ID NO: 22) are used to amplify the Acinetobacter baumannii aminoglycoside phosphotransferase enzyme APH(3′)-VI from plasmid DNA p72-psbA-aphA6 (Bateman et al., Mol. Gen. Genet. 263:404-410; 2000). Amplification is performed with a Pfx proof reading enzyme (Accuprime Pfx, Invitrogen, Carlsbad, Calif.) using the following conditions: 95° C. 5 min, (94° C. 45 sec, 55° C. 60 sec, 68° C. 90 sec) for 25 cycles, 68° C. 7 min. The PCR product is digested with NcoI and PstI and the resulting 801 base pair fragment is ligated into the NcoI and PstI sites of pDs69r, producing vector “pDs69r-A6” (FIG. 7).

The chloramphenicol acetyltransferase gene, CAT, of Escherichia coli transposon Tn9 is PCR amplified with forward primer 5′ cgttacgtatcggatcc3′ (SEQ ID NO: 89) and reverse primer 5′ctaggctcgagaagcttttacgccccgccctgc3′ (SEQ ID NO: 90) from plasmid pACYC184 (New England Biolabs, Beverly, Mass.) digested with BamHI and HindIII, and ligated into the BamHI and HindIII sites of the multipurpose cloning vector pSTBlue1 (EMD Chemicals, Inc. San Diego, Calif.). The CAT gene is subjected to XhoI, partial NcoI digestion, and the 668 base pair product is cloned into the NcoI and XhoI sites of pDS69r, producing vector “pDs69r-CAT”. Using this general strategy, additional Dunaliella and Tetraselmis vectors may be generated based on the sequence database obtained from Examples 1 and 2.

Following is the aadA gene sequence plus 5′ NcoI and 3′ PstI and SphI restriction sites added in PCR cloning:

(SEQ ID NO: 23)

ccatggctcgtgaagcggtgatcgccgaagtatcgactcaactatcagag
gtagttggcgtcatcgagcgccatctcgaaccgacgttgctggccgtaca
tttgtacggctccgcagtggatggcggcctgaagccacacagtgatattg
atttgctggttacggtgaccgtaaggcttgatgaaacaacgcggcgagct
ttgatcaacgaccttttggaaacttcggcttcccctggagagagcgagat
tctccgcgctgtagaagtcaccattgttgtgcacgacgacatcattccgt
ggcgttatccagctaagcgcgaactgcaatttggagaatggcagcgcaat
gacattcttgcaggtatcttcgagccagccacgatcgacattgatctggc
tatcttgctgacaaaagcaagagaacatagcgttgccttggtaggtccag
cggcggaggaactctttgatccggttcctgaacaggatctatttgaggcg
ctaaatgaaaccttaacgctatggaactcgccgcccgactgggctggcga
tgagcgaaatgtagtgcttacgttgtcccgcatttggtacagcgcagtaa
ccggcaaaatcgcgccgaaggatgtcgctgccgactgggcaatggagcgc
ctgccggcccagtatcagcccgtcatacttgaagctagacaggcttatct
tggacaagaagaagatcgcttggcctcgcgcgcagatcagttggaagaat
ttgtccactacgtgaaaggcgagatcaccaaggtagtcggcaaataactg
caggcatgc

Following is the aphA6 gene sequence plus 5′ NcoI and 3′ PstI restriction sites added in PCR cloning:

(SEQ ID NO: 24)

ccatggaattaccaaatattattcaacaatttatcggaaacagcgtttta
gagccaaataaaattggtcagtcgccatcggatgtttattcttttaatcg
aaataatgaaactttttttcttaagcgatctagcactttatatacagaga
ccacatacagtgtctctcgtgaagcgaaaatgttgagttggctctctgag
aaattaaaggtgcctgaactcatcatgacttttcaggatgagcagtttga
attcatgatcactaaagcgatcaatgcaaaaccaatttcagcgctttttt
taacagaccaagaattgcttgctatctataaggaggcactcaatctgtta
aattcaattgctattattgattgtccatttatttcaaacattgatcatcg
gttaaaagagtcaaaattttttattgataaccaactccttgacgatatag
atcaagatgattttgacactgaattatggggagaccataaaacttaccta
agtctatggaatgagttaaccgagactcgtgttgaagaaagattggtttt
ttctcatggcgatatcacggatagtaatatttttatagataaattcaatg
aaatttattttttagatcttggtcgtgctgggttagcagatgaatttgta
gatatatcctttgttgaacgttgcctaagagaggatgcatcggaggaaac
tgcgaaaatatttttaaagcatttaaaaaatgatagacctgacaaaagga
attattttttaaaacttgatgaattgaattgattccaagcattatctaaa
atactctgcag

Following is the cat gene sequence plus 5′ NcoI and 3′ XhoI restriction sites added in PCR cloning:

(SEQ ID NO: 25)

ccatggagaaaaaaatcactggatataccaccgttgatatatcccaatgg
catcgtaaagaacattttgaggcatttcagtcagttgctcaatgtaccta
taaccagaccgttcagctggatattacggcctttttaaagaccgtaaaga
aaaataagcacaagttttatccggcctttattcacattcttgcccgcctg
atgaatgctcatccggaattccgtatggcaatgaaagacggtgagctggt
gatatgggatagtgttcacccttgttacaccgttttccatgagcaaactg
aaacgttttcatcgctctggagtgaataccacgacgatttccggcagttt
ctacacatatattcgcaagatgtggcgtgttacggtgaaaacctggccta
tttccctaaagggtttattgagaatatgtttttcgtctcagccaatccct
gggtgagtttcaccagttttgatttaaacgtggccaatatggacaacttc
ttcgcccccgttttcaccatgggcaaatattatacgcaaggcgacaaggt
gctgatgccgctggcgattcaggttcatcatgccgtttgtgatggcttcc
atgtcggcagaatgcttaatgaattacaacagtactgcgatgagtggcag
ggcggggcgtaaaagcttctcgag

### EXAMPLE 8

This example illustrates one possible method for introduction of gene sequences into vectors for targeted integration of DNA segments in the chloroplast genome.

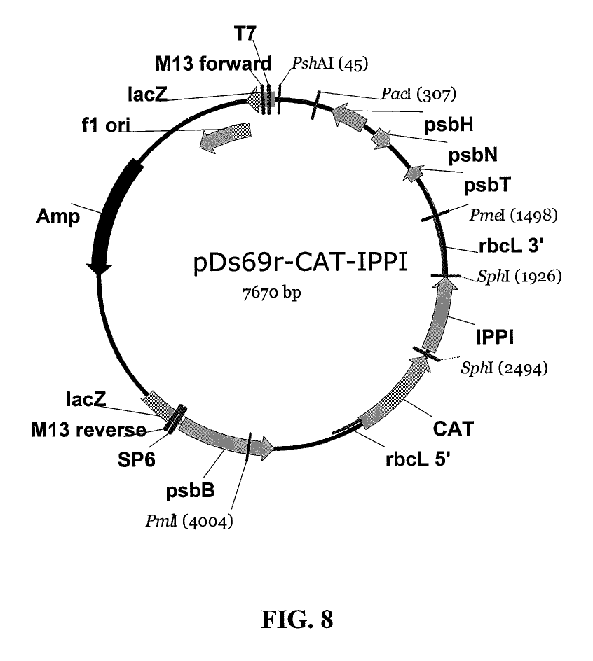

Targeted integration segments can be used, for example, to facilitate nucleic acid variation that manifests introduction of genes into the chloroplast that participate in isoprenoid biosynthesis, such as IPPI. One specific embodiment exemplifies a chloroplast cassette, pDs69r-CAT-IPPI (FIG. 8), in which the nucleic acid encodes the gene Isopentenyl Pyrophosphate Isomerase, IPPI (F. Hahn, et al., U.S. Pat. No. 7,129,392; 2006, which is incorporated herein by reference in its entirety). The IPPI gene of Rhodobacter capsulatus is PCR amplified from Rhodobacter genomic DNA with the addition of terminal restriction sites for the enzyme SphI (GCATGC) by use of primers forward ′CTTTATAGAGCATGCGATTCCCATTAGGAGGTAGTACCAAATGGCCGAGGAGA TGATCCCCGC3′ (SEQ ID NO: 26) and reverse 5′GCGCGCCGCATGCGAGCTCTCAGGCCGTCACCGGCGGAAAGATC3′ (SEQ ID NO: 27). Amplification is performed with a Pfx proof reading enzyme (Accuprime Pfx, Invitrogen, Carlsbad, Calif.) using the following conditions; 95° C. 3 min, (94° C. 30 sec, 55° C. 60 sec, 72° C. 40 sec) for 25 cycles, 72° C. 7 min. The resulting 590 base pair product is digested with SphI and ligated into the SphI site of pDs69r-CAT, producing vector pDs69r-CAT-IPPI. Using this general strategy, additional Dunaliella and Tetraselmis vectors may be generated based on the sequence database obtained from Examples 1 and 2.

Following is the Rhodobacter IPPI gene sequence plus 5′ and 3′ SphM restriction sites added in PCR cloning:

(SEQ ID NO: 28)

gcatgcgattcccattaggaggtagtaccaaatggccgaggagatgatcc
ccgcctgggtcgagggcgtgctgcaacccgtcgagaagctggaggcccac
cgcaagggcctgcggcatctggcgatttcggtcttcgtgacgcgcggcaa
caaggtgcttttgcagcaacgcgcgctgtcgaaatatcacacgccggggc
tttgggcgaatacctgctgcacccatccctattggggcgaggatgcgccg
acctgcgccgcccgccgtctggggcaggagctgggcatcgtcgggctgaa
gctgcgccacatggggcagctggaataccgcgccgatgtgaacaacggca
tgatcgagcatgaggtggtggaggtcttcaccgccgaagcgcccgagggg
atcgagccgcaacccgaccccgaggaagtggccgataccgaatgggtgcg
catcgacgcgctgcgctcggagatccacgccaatccggaacgcttcacgc
cctggctcaagatctatatcgagcagcaccgcgacatgatctttccgccg
gtgacggcctgagagctcgcatgc

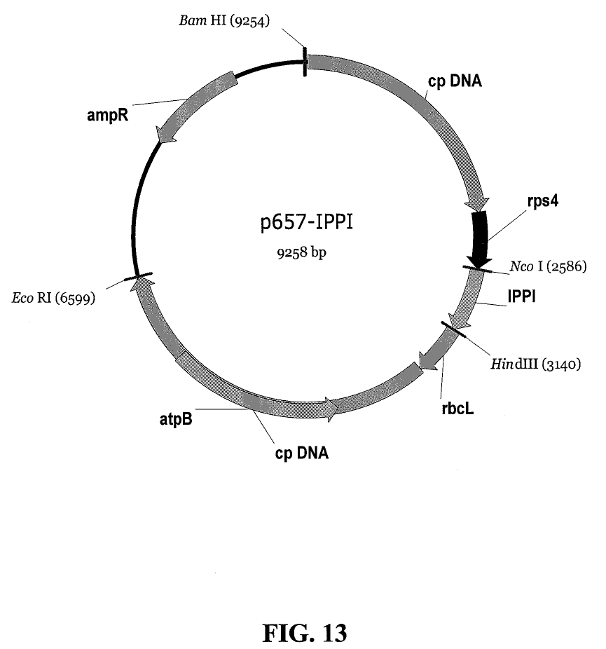

Another specific embodiment exemplifies a chloroplast cassette, p657-IPPI (FIG. 13), in which the nucleic acid encodes the gene Isopentenyl Pyrophosphate Isomerase, IPPI. The IPPI gene of Rhodobacter capsulatus is PCR amplified from Rhodobacter genomic DNA with the addition of terminal restriction sites for NcoI by the use of primers forward

(SEQ ID NO: 61)

5′ ctttatagaccatggaggcaaaccttatggccgaggagatg 3′
and HindIII by the use of primers reverse

(SEQ ID NO: 62)

5′ ccttgagaagcttgcatgctcaggccgtcaccggcgg 3′

Amplification is performed with a Pfx proof reading enzyme (Accuprime Pfx, Invitrogen, Carlsbad, Calif.) using the following conditions; 95° C. 3 min, (94° C. 30 sec, 55° C. 60 sec, 72° C. 40 sec) for 25 cycles, 72° C. 7 min. The resulting 576 base pair product is digested with NcoI and HindIII and ligated into the NcoI and HindIII sites of p657, producing vector p657-IPPI. Using this general strategy, additional Chlamydomonas-type vectors may be generated.

Following is the PCR amplified product including the Rhodobacter IPPI gene sequence after restriction digestion with NcoI and HindIII:

(SEQ ID NO: 63)

catggaggcaaaccttatggccgaggagatgatccccgcctgggtcgagg
gcgtgctgcaacccgtcgagaagctggaggcccaccgcaagggcctgcgg
catctggcgatttcggtcttcgtgacgcgcggcaacaaggtgcttttgca
gcaacgcgcgctgtcgaaatatcacacgccggggctttgggcgaatacct
gctgcacccatccctattggggcgaggatgcgccgacctgcgccgcccgc
cgtctggggcaggagctgggcatcgtcgggctgaagctgcgccacatggg
gcagctggaataccgcgccgatgtgaacaacggcatgatcgagcatgagg
tggtggaggtcttcaccgccgaagcgcccgaggggatcgagccgcaaccc
gaccccgaggaagtggccgataccgaatgggtgcgcatcgacgcgctgcg
ctcggagatccacgccaatccggaacgcttcacgccctggctcaagatct
atatcgagcagcaccgcgacatgatctttccgccggtgacggcctgagca
tgca

Yet another specific embodiment exemplifies a chloroplast cassette, pDs69r-CAT-SyIPPI. The IPPI gene of Synechocystis sp. PCC6803 PCR is amplified from Synechocystis genomic DNA with the addition of terminal restriction sites for the enzyme BspHI (TCATGA) by use of primers forward 5′ TAC CTC ATG ACC TAG CAG CAC CAC CAC AAT ATG C 3′ (SEQ ID NO: 64) and the enzyme SphI (GCATGC) by use of primers reverse: 5′ AAT CGC ATG CGG TTA AAC CGA GGG GAT GAT GTA C 3′ (SEQ ID NO: 91) The resulting 1345 base pair product includes 118 base pairs of adjacent 5′ UTR:

(SEQ ID NO: 65)

5′cctagcagcaccaccacaatatgcccccaccttaatcctgggttattt
ttaagttattgctccactccctccagttgatggcaaaattgcttgccggt
atttgtaatgtaattcactg3′

and 167 bp of adjacent 3′ UTR:

(SEQ ID NO: 66)

5′gggacattttgctctggttgacgatacagtgaagcttggactggttga
ccccgatagctgcggagtagggcatcaagccacagttttcctttaataat
ccccccatgaaatggcataaagagagcaaagtattactacaaggagtaca
tcatcccctcggtttaacc3′

The PCR product is digested with BspHI and SphI and ligated into the SphI site of pDs69r-CAT, producing vector pDs69r-CAT-SyIPPI.

Following is the Synechocystis sp. PCC6803 IPPI gene PCR fragment including 5′ UTR and 3′ UTR sequences after digestion with BspHI and SphI:

(SEQ ID NO: 67)

5′catgacctagcagcaccaccacaatatgcccccaccttaatcctgggt
tatttttaagttattgctccactccctccagttgatggcaaaattgcttg
ccggtatttgtaatgtaattcactgatggatagcaccccccaccgtaagt
ccgatcatatccgcattgtcctagaagaagatgtggtgggcaaaggcatt
tccaccggctttgaaagattgatgctggaacactgcgctcttcctgcggt
ggatctggatgcagtggatttgggactgaccctctggggtaaatccttga
cttacccttggttgatcagcagtatgaccggcggcacgccagaggccaag
caaattaatctatttttagccgaggtggcccaggctttgggcatcgccat
gggtttgggttcccaacgggccgccattgaaaatcctgatttagccttca
cctatcaagtccgctccgtcgccccagatattttactttttgccaacctg
ggattagtgcaattaaattacggttacggtttggagcaagcccagcgggc
ggtggatatgattgaagccgatgcgctgattttgcatctcaatcccctcc
aggaagcggtgcaacccgatggcgatcgcctgtggtcgggactctggtct
aagttagaagctttagtagaggctttggaagtgccggtaattgtcaaaga
agtgggcaatggcattagcggtccggtggccaaaagattgcaggaatgtg
gggtcggggcgatcgatgtggctggagctgggggcaccagttggagtgaa
gtggaagcccatcgacaaaccgatcgccaagcgaaggaagtggcccataa
ctttgccgattggggattacccacagcctggagtttgcaacaggtagtgc
aaaatactgagcagatcctggttttcgccagcggcggcattcgttccggc
attgacggggccaaggcgatcgccctgggggccaccctggtgggtagtgc
ggcaccggtattagcagaagcgaaaatcaacgcccaaagggtttatgacc
attaccaggcacggctaagggaactgcaaatcgccgccttttgttgtgat
gccgccaatctgacccaactggcccaagtccccctttgggacagacaatc
gggacaaaggttaactaaaccttaagggacattttgctctggttgacgat
acagtgaagcttggactggttgaccccgatagctgcggagtagggcatca
agccacagttttcctttaataatccccccatgaaatggcataaagagagc
aaagtattactacaaggagtacatcatcccctcggtttaaccgcatg3′

Using this general strategy, additional Dunaliella, Tetraselmis or other host vectors may be generated.

### EXAMPLE 9

This example pertains to a protein that participates in fatty acid biosynthesis, acetyl-coA carboxylase, specifically one or more of its heteromeric subunits: biotin carboxylase (BC), biotin carboxyl carrier protein (BCCP), α-carboxyltransferase (α-CT), β-carboxyltransferase (β-CT). This example embodies a targeted integration segment in which the nucleic acid encodes the gene, AccD. Chloroplast genome sequencing has shown that some green algae have the accD gene of the heteromeric acetyl-CoA carboxylase enzyme (ACCase) located in the chloroplast, similar to that found in dicots. The other ACCase genes, designated accA, accB, and accC, are encoded in the nuclear genome. AccD encodes the beta subunit of the carboxyltransferase component of the E. coli acetyl-CoA carboxylase for catalyzing the first committed step in fatty acid biosynthesis (S J Li and J E Cronan, J. Biol. Chem. 267: 16841-16847; 1992); in Dunaliella it appears to be encoded in the nucleus (GenBank #EF363909; Unpublished direct submission to GenBank: Liang, X Z, Li, G. and Yang, Z R. (2007) The cloning of acetyl-coenzyme A carboxylase carboxyl transferase subunit beta from Dunaliella salina). The Chlorella accD gene (Genbank accession #NC_{—}001865) is used as a first example for construction of pDs69r-CAT-accD. The freshwater Chlorella chloroplast has been completely sequenced (Wakasugi T, et al., Proc Natl Acad Sci USA 94: 5967-5972; 1997).

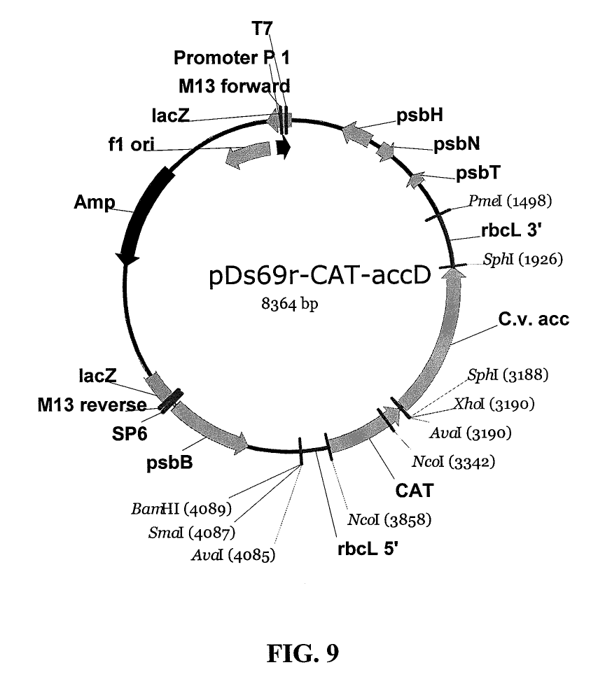

Primers Cv-accD1 5′-CAAATTGCATGCGGAGGACTACTTATTATGTCAATTCTTTCTTGGATCGA-3′ (SEQ ID NO: 29) and Cv-accD2 5′-TAGGTAGCATGCATTAGCTAAAATTTTGGTCTAATTCGAAATTCTG-3′ (SEQ ID NO: 30) are used. Amplification is performed with a Pfx proof reading enzyme from a genomic DNA preparation of Chlorella vulgaris using the following conditions: 95° C. 4 min, (94° C. 30 sec, 53° C. 30 sec, 68° C. 90 sec) for 25 cycles, 68° C. 7 min. After amplification, the resulting gene product (1280 bp) is digested and cloned into the SphI restriction site of pDs69r-CAT. The resulting vector, “pDs69r-CAT-accD” (FIG. 9), contains a cassette consisting of the D. salina rbcL promoter, chloramphenicol transacetylase (CAT) gene, a ribosome binding site, the accD gene and the rbcL terminator, all surrounded by D. salina chloroplast sequence for homologous integration. The methodology is directly applicable to use of the D. salina accD for expression in the chloroplast. Using this general strategy, additional Dunaliella and Tetraselmis vectors may be generated.

Following is the sequence of the Chlorella accD gene plus SphI restriction sites added in PCR cloning:

(SEQ ID NO: 31)

CAAATTGCATGCGGAGGACTACTTATTatgtcaattc tttcttggat
cgaaaatcaa cgaaaattga aattattaaa tgcacctaaa
tacaatcatc cagagtcaga cgtaagtcaa ggtctttgga
cacgctgcga ccattgtggt gtaatattat atattaaaca
tttaaaagaa aaccaacgtg tatgttttgg ttgcggatat
catctacaaa tgagtagtac agaacgaatt gagtcactag
ttgatgcaaa tacgtggcgt ccctttgatg aaatggtgtc
accatgtgat ccattagaat ttcgagatca aaaagcctat
acagaaagat taaaagacgc acaagaacga acaggtctgc
aagatgctgt tcaaacagga acaggacttc ttgacggtat
tccgatagcc ttaggagtta tggattttca ttttatgggg
ggaagtatgg gctctgtagt tggtgaaaaa atcacgcgtt
taatagaata cgcaactcaa gaaggtttac ccgtaatttt
agtttgtgct tctggcggag ctcgaatgca agaaggtatt
ttaagcttaa tgcaaatggc aaaaatttct gccgctcttc
atattcacca aaattgcgcc aaattacttt atatttcagt
cttaacttca ccaacaacag gtggtgtaac tgctagcttt
gctatgttag gggatcttct ttttgcagaa ccaaaagctt
taattgggtt tgctggtcgt cgggtgattg aacaaacctt
acaagagcaa ttacctgatg attttcaaac tgctgagtat
ttgttacatc atggtcttct tgatttaatc gtaccacgat
cttttttaaa acaagcttta tctgaaaccc taacacttta
taaagaagct ccgttaaaag aacagggtcg gattccttat
ggtgaacgtg ggcctcttac aaaaactcgt gaagaacaac
ttcgtcggtt tcttaaatcg tcaaaaactc ctgaatattt
acatattgta aatgatttaa aagaattact tggtttttta
ggtcaaactc agaccactct ttaccctgaa aaactggaat
ttttaaataa cctaaaaacc caagaacagt ttctacaaaa
aaatgataat ttttttgaag agcttttaac ttcaacaaca
gtaaaaaaag ctttgaattt agcttgtgga acacaaaccc
gtctgaattg gcttaattat aagttaacag aatttcgaat
tagaccaaaa ttt tagCTAATGCATGCTACCTA

### EXAMPLE 10

This example embodies a targeted integration segment in which the nucleic acid encodes a gene that participates in fatty acid biosynthesis, acyl-ACP thioesterase.

Fatty acid carbon chain elongation occurs in the chloroplast, with a covalently-bound acyl carrier protein attached to the carbon chain. Export of the growing carbon chain from the chloroplast to the cytosol is prevented until removal of the acyl carrier protein is accomplished by the activity of acyl carrier protein thioesterase (ACPTE). At least two types of ACPTE have been identified and classified based upon preference for long- or medium-chain carbon chain substrates (Jones A, et al., Plant Cell 7:359-371; 1995). Medium-chain specific thioesterases (FatB) are less stringent than long-chain thioesterases (FatA), with activity ranging from 8:0/10:0 fatty acids (Dehesh K, et al., Plant J. 9(2):167-172; 1996) to 12:0/14:0 fatty acids (Voelker T and Davies H. J. Bacteriol. 176:7320-7327; 1994). The heterologous expression of a medium-chain ACPTE in E. coli or Brassica effectively alters the resulting fatty acid profile of the transgenic organism, shifting the predominant free fatty acid toward the shorter chain length preferred by the thioesterase as a substrate.

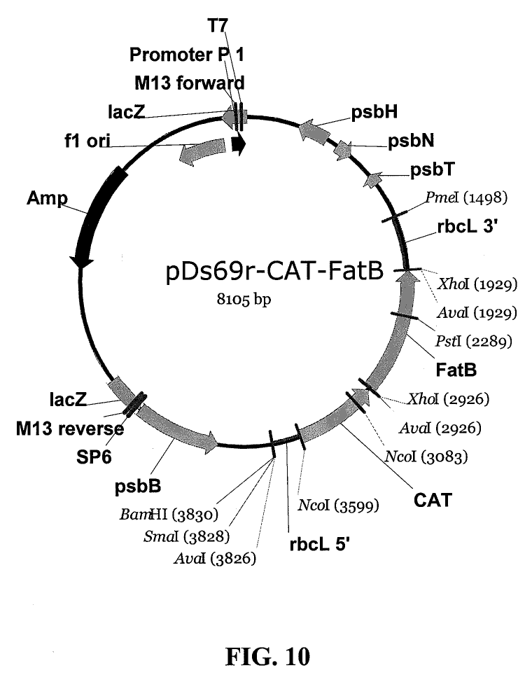

Primers 5′ctttatagactcgagaggaggaaaaaagtacatgttgcctgactggagcatgctctttgcagtg3′ (SEQ ID NO: 32) and 5′gcgcgccctcgagttacaccctcggttctgcgggtatcacactaat3′ (SEQ ID NO: 33) are used to amplify a cDNA encoding the mature peptide form of Umbellularia californica 12:0 acyl-ACP thioesterase from total cDNA. This coding sequence lacks the signal peptide that is no longer needed to target the protein to the chloroplast. The nucleotide product includes a ribosome-binding site to facilitate translation of the protein. Amplification is performed with a Pfx proofreading enzyme using the following conditions: 95° C. 3 min, (94° C. 30 sec, 58° C. 60 sec, 72° C. 40 sec) for 25 cycles, 72° C. 7 min. The 953 base pair product is digested with XhoI and ligated into the XhoI site of pDs69r-CAT, producing vector “pDs69r-CAT-FatB” (FIG. 10).

Degenerate PCR amplification of the Dunaliella or Tetraselmis ACPTE can be used to clone and express the homologous gene in host cells to achieve a desired phenotype.

A list of known FatB genes is compiled for identification of conserved motifs for primer design: Arabidopsis thaliana FATB NM-100724; California Bay Tree thioesterase M94159; Cuphea hookeriana 8:0- and 10:0-ACP specific thioesterase (FatB2) U39834; Cinnamomum camphora acyl-ACP thioesterase U31813; Diploknema butyracea chloroplast palmitoyl/oleoyl specific acyl-acyl carrier protein thioesterase (FatB) AY835984; Madhuca longifolia chloroplast stearoyl/oleoyl specific acyl-acyl carrier protein thioesterase precursor (FatB) AY835985; Populus tomentosa FATB DQ321500; and Umbellularia californica Uc FatB2 UCU17097.

To clone FatB genes from microalgae, isolation of total and poly (A)⁺ RNA is performed. Algal cultures are harvested by centrifugation at 3000×g for 10 minutes. The cell pellet is transferred to a mortar and pestle and ground to a fine powder under liquid nitrogen. The frozen ground material is transferred to a polypropylene tube and suspended in 5 mL of TriPure Isolation Reagent (Roche). Total RNA is isolated using the manufacturer's protocol. Poly (A)⁺ RNA is then prepared with an mRNA isolation kit (Amersham Pharmacia Biotech). Next, cDNA library construction and screening is performed. cDNA synthesis is accomplished with the cDNA Synthesis Kit (Stratagene). cDNA is purified on a Sephacryl S-400 Spin Column (Amersham Pharmacia Biotech) and extracted with phenol:chloroform:isoamyl alcohol. The aqueous cDNA-containing supernate is ethanol precipitated and resuspended in TE buffer. The cDNA is cloned into the Topo Shotgun Cloning Vector (Invitrogen) and the resulting library is amplified and stored at −20° C. until screening. The E. coli library is plated at about 500 clones per 150 mm Petri dish, blotted to nylon membranes and screened FatB genes using DNA probes synthesized by degenerate PCR.

Probes for FatB are designed using degenerate PCR primers based on three conserved motifs of FatB: Motif “W”: YPT/AWGDT/VV (SEQ ID NO: 34); motif “Q”: “WNDLDVNQHV” (SEQ ID NO: 35); and motif “C”: EYRREC (SEQ ID NO: 36). They are used in a combinatorial manner with total mRNA template prepared as outlined above to produce three cDNA probes of varying approximate lengths: Wₛₑₙₛₑ (5′TAYCCIRCITGGGGIGAYRYIGTI3′) (SEQ ID NO: 37) and Qₐₙₜᵢₛₑₙₛₑ (5′ACRTGYTGRTTIACRTCIARRTCRTTCCAI3′) (SEQ ID NO: 38), product 330 base pairs; Qₛₑₙₛₑ (5′TGGAAYGAYYTIGAYGTIAAYCARCAYGTI3′) (SEQ ID NO: 39) and Cₐₙₜᵢₛₑₙₛₑ (5′CAYTCICKICKRTAYTCI3′) (SEQ ID NO: 40), product 129 base pairs; Wₛₑₙₛₑ (5′TAYCCIRCITGGGGIGAYRYIGTI3′) (SEQ ID NO: 41) and Cₐₙₜᵢₛₑₙₛₑ (5′CAYTCICKICKRTAYTCI3′) (SEQ ID NO: 42), product 432 base pairs. For the cDNA probe sequences, I=inosine, R=A or G, Y=C or T, M=A or C, K=G or T, S=C or G, W=A or T, H=A, C or T, B=C, G or T, V=A, C or G, D=A, G or T, and N=A, C, G or T. PCR conditions for probe synthesis using Accuprime Pfx DNA Polymerase (Invitrogen) are: initial denaturation at 94° C. for 3 min; four cycles of 94° C. for 15 sec, 52° C. for 30 sec and 72° C. for 45 sec; 10 cycles of 94° C. for 15 sec, 52° C. (decreasing by 1° C. per cycle) for 30 sec, 72° C. for 45 sec; 25 cycles of 94° C. for 15 sec, 42° C. for 30 sec, and 72° C. for 45 sec (increasing by 3 sec per cycle); final extension step of 72° C. for 6 min. Probes are labeled and library membranes are hybridized using the North2South Kit (Pierce). Positive clones are identified by hybridization, amplified, and sequenced for identification of the hybridizing DNA insert containing the FatB homologue. Library screening and sequencing continues until the 5′ and 3′ ends of the mRNA have been identified and a full-length clone is obtained. Using this general strategy, additional Dunaliella and Tetraselmis vectors may be generated based on the sequence database obtained from Examples 1 and 2.

Following is the nucleic acid sequence encoding the Umbellularia californica acyl-ACP thioesterase mature protein (no signal peptide), plus XhoI restriction sites added in PCR cloning:

(SEQ ID NO: 43)

ctttataga c tcgagaggaggaaaaaagtacatg ttgcct gac
tggagcatgc tctttgcagt gatcacaacc atcttttcgg
ctgctgagaa gcagtggacc aa tctagagt ggaagccgaa
gccgaagcta ccccagttgc ttgatgacca ttttggactg
catgggttag ttttcaggcg cacctttgcc atcagatctt
atgaggtggg acctgaccgc tccacatcta tactggctgt
tatgaatcac atgcaggagg ctacacttaa tcatgcgaag
agtgtgggaa ttctaggaga tggattcggg acgacgctag
agatgagtaa gagagatctg atgtgggttg tgagacgcac
gcatgttgct gtggaacggt accctacttg gggtgatact
gtagaagtag agtgctggat tggtgcatct ggaaataatg
gcatgcgacg tgatttcctt gtccgggact gcaaaacagg
cgaaattctt acaagatgta ccagcctttc ggtgctgatg
aatacaagga caaggaggtt gtccacaatc cctgacgaag
ttagagggga gatagggcct gcattcattg ataatgtggc
tgtcaaggac gatgaaatta agaaactaca gaagctcaat
gacagcactg cagattacat ccaaggaggt ttgactcctc
gatggaatga tttggatgtc aatcagcatg tgaacaacct
caaatacgtt gcctgggttt ttgagaccgt cccagactcc
atctttgaga gtcatcatat ttccagcttc actcttgaat
acaggagaga gtgcacgagg gatagcgtgc tgcggtccct
gaccactgtc tctggtggct cgtcggaggc tgggttagtg
tgcgatcact tgctccagct tgaaggtggg tctgaggtat
tgagggcaag aacagagtgg aggcctaagc ttaccgatag
tttcagaggg attagtgt ga tacccgcaga accgagggtg taa c
tcgag ggcgcgc

### EXAMPLE 11

This example embodies a targeted integration segment for the chloroplast genome in which the nucleic acid encodes a gene that participates in fatty acid biosynthesis, acetyl-coA synthetase (ACS).

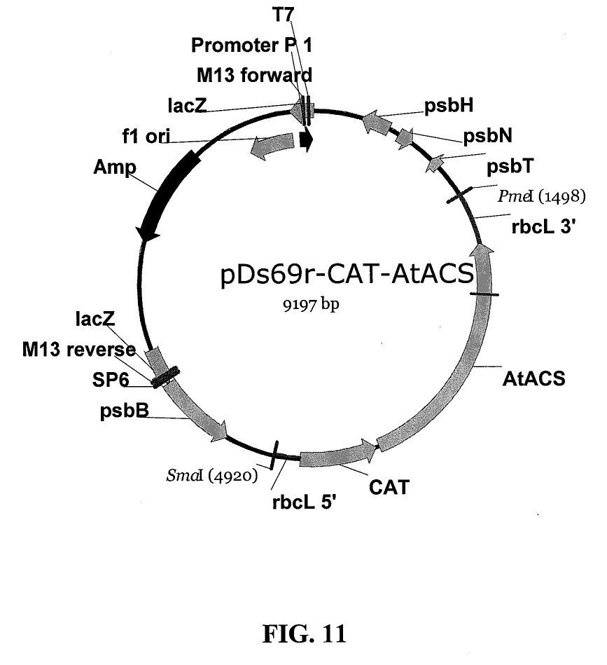

Primers 5′ctttatagagtcgacctagaagtgaaagatgattccttatgctgctggtgttattgtg 3′ and 5′gcgcgccgtcgacftaggcatataacttggtgagatcttcagagaattc 3′ are used to amplify a cDNA encoding Acetyl Coenzyme A Synthetase from Arabidopsis thaliana cDNA. Amplification is performed with a Pfx proofreading enzyme using the following conditions; 95° C. 3 min, (94° C. 30 sec, 58° C. 60 sec, 72° C. 40 sec) for 25 cycles, 72° C. 7 min. The 953 base pair product is digested with SalI and ligated into the XhoI site of pDs69r-CAT, producing vector “pDs69r-CAT-AtACS” (FIG. 11).

ACS genes can also be cloned from microalgae. Degenerate PCR amplification of the Dunaliella or Tetraselmis ACS is desired for homologous gene expression in the chloroplast, which is as or more effective than heterologous expression of Arabidopsis or like genes. This commences with cDNA library construction and screening as described in Example 10.

Primer design can be based on any number of closely related ACS genes by those skilled in the art using for example Arabidopsis ACS9 gene GI:20805879; Brassica napus ACS gene GI: 12049721; Oryza sativa ACS gene GI: 115487538; or Trifolium pratense ACS gene GI:84468274. Probes for ACS use degenerate PCR primers designed based on three conserved motifs of ACS: Motif G: “GDTQRFINIC” (SEQ ID NO: 44); motif K: “KKDIVKLQHGEYV” (SEQ ID NO: 45); and motif P: EKFEIPAKIK (SEQ ID NO: 46). They are used in a combinatorial manner with total mRNA template prepared as outlined in example 10 to produce three cDNA probes of varying lengths: Gₛₑₙₛₑ (5′GGIGAYACICARMGITTYATIAAYATITGYI3′) (SEQ ID NO: 47) and Kₐₙₜᵢₛₑₙₛₑ (5′ACRTAYTCRTGYTGIARIACDATRTCYTTYTTI3′) (SEQ ID NO: 48), product approximately 405 base pairs; Kₛₑₙₛₑ (5′AARAARGAYATHGTIYTICARCAYGARTAYGTI3′) (SEQ ID NO: 49) and Pₐₙₜᵢₛₑₙₛₑ (5′TTDATYTTIGGDATYTCRAAYTTYTCI3′) (SEQ ID NO: 50), product approximately 306 base pairs; Gₛₑₙₛₑ (5′GGIGAYACICARMGITTYATIAAYATITGYI3′) (SEQ ID NO: 51) and Pₐₙₜᵢₛₑₙₛₑ (5′TTDATYTTIGGDATYTCRAAYTTYTCI3′) (SEQ ID NO: 52), product approximately 675 base pairs. For the cDNA probe sequences, I=inosine, R=A or G, Y=C or T, M=A or C, K=G or T, S=C or G, W=A or T, H=A, C or T, B=C, G or T, V=A, C or G, D=A, G or T, and N=A, C, G or T. PCR conditions for probe synthesis using Accuprime Pfx DNA Polymerase (Invitrogen) are: initial denaturation at 94° C. for 3 min; four cycles of 94° C. for 15 sec, 52° C. for 30 sec and 72° C. for 45 sec; 10 cycles of 94° C. for 15 sec, 52° C. (decreasing by 1° C. per cycle) for 30 sec, 72° C. for 45 sec; 25 cycles of 94° C. for 15 sec, 42° C. for 30 sec, and 72° C. for 45 sec (increasing by 3 sec per cycle); final extension step of 72° C. for 6 min. The PCR products are labeled and algae cDNA library membranes are hybridized using the North2South Kit (Pierce). Positive clones are identified by hybridization, amplified, and sequenced for identification of the hybridizing DNA insert. Library screening and sequencing continues until the 5′ and 3′ ends of the mRNA have been identified and a full-length clone is obtained. Using this general strategy, additional Dunaliella and Tetraselmis vectors may be generated based on the sequence database obtained from Examples 1 and 2.

Following is the sequence of Arabidopsis thaliana long chain acyl-CoA synthetase 9 (LACS9) mRNA (AF503759 2076 bp mRNA):

(SEQ ID NO: 53)

atgattcctt atgctgctgg tgttattgtg ccattggctt
tgacgtttct ggttcagaaa tctaagaaag aaaagaaaag
aggtgttgtt gttgatgttg gtggtgaacc aggttatgct
attaggaatc acaggtttac tgagcctgtt agttcccatt
gggaacatat ctcaacgctt ccagagctct ttgagatatc
gtgtaatgct cacagtgata gggttttcct tggcacccga
aagctgatct ctagagagat tgagactagt gaggatggaa
aaacgttcga gaaactgcat ttaggtgact acgagtggct
cacttttggg aagactctcg aagcagtgtg tgattttgcc
tctgggttag ttcagattgg gcacaagacg gaagagcgtg
tcgccatttt tgcagatact agagaagaat ggttcatctc
cctacagggt tgcttcaggc gcaacgtcac tgtggtaact
atctattcat ctttgggaga ggaagctctt tgtcactcgc
tgaatgagac agaggtcaca accgtaatat gtggtagcaa
agaactcaaa aagctcatgg acataagcca acagcttgaa
actgtgaaac gtgtgatatg catggatgat gaattcccat
ctgatgtgaa cagtaattgg atggcgactt catttactga
tgttcagaaa cttggccgcg aaaatcctgt ggatcctaat
ttccctctct cagcagatgt tgctgttata atgtacacca
gtggaagcac tggacttccc aagggtgtta tgatgacgca
tggtaatgtc ctagctacag tttcggcagt gatgacaatt
gttcctgacc ttggaaagag ggatatatac atggcatatt
tacctttggc tcacatcctt gagttagcag ctgagagcgt
aatggctact attgggagtg ctattggata tgggtctccc
ttgacgctaa cggatacttc aaacaagata aaaaagggta
caaaaggaga tgtcacagca ctaaagccca ctataatgac
agctgttcca gccattcttg atcgtgtcag ggatggtgtc
cgcaaaaagg ttgatgcaaa gggcggattg tcaaagaaat
tgtttgactt tgcatatgct cggcgattat ctgcaatcaa
tggaagttgg tttggagcct ggggattgga aaagcttttg
tgggatgtgc ttgtgttcag gaaaatccgt gcagttttgg
gaggtcaaat ccgctatttg ctctctggtg gtgcccctct
ttctggtgac actcagagat tcattaacat ctgcgttggg
gctccaatcg gtcagggata tgggctcaca gagacttgtg
ctggtggaac cttctcggag tttgaggaca catccgttgg
ccgtgttggt gctccacttc cttgctcctt tgtaaagcta
gtagactggg cggaaggtgg gtatctaact agtgataagc
cgatgccccg tggtgaaatt gtaattggtg gctcaaatat
cacgcttggg tatttcaaaa atgaggagaa aactaaagaa
gtgtacaagg ttgatgaaaa gggaatgagg tggttctaca
caggagacat aggacgattt caccctgatg gctgcctcga
gataatagac cgaaaaaagg atatcgttaa acttcagcat
ggagaatatg tctccttggg caaagttgaa gctgctctaa
gtataagtcc ctatgttgaa aacataatgg ttcatgctga
ttcgttctac agttactgtg tggctcttgt ggtcgcgtcc
caacatacag ttgaaggttg ggcttcaaag caaggaatag
actttgccaa cttcgaagaa ctgtgcacga aagagcaagc
cgtgaaagaa gtgtatgcgt cccttgtgaa ggcggctaaa
caatcacgat tggagaagtt tgagatacca gcaaagatca
aattattggc atctccatgg acgccagagt caggattagt
cacagcagct ctaaagctga aaagagatgt aattaggagg
gaattctctg aagatctcac caagttatat gcctaa

In some embodiments ACC synthetase and ACC carboxylase are co-expressed to preferentially form acetyl co-A. In some embodiments the transformed host cells are grown under non-carbon limiting conditions or carbon-enriched conditions.

### EXAMPLE 12

This example embodies targeted integration segments for the chloroplast in which the nucleic acid encodes a gene that participates in fatty acid biosynthesis via the pyruvate dehydrogenase complex, including one or more of the following subunits that comprise the complex: Pyruvate dehydrogenase E1α; Pyruvate dehydrogenase E1β; dihydrolipoamide acetyltransferase; dihydrolipoamide dehydrogenase. The pyruvate dehydrogenase complex plays a key role in chloroplast carbon metabolism and de novo synthesis of fatty acids due to its enzymatic function catalyzing the production of acetyl-CoA and NADH via oxidative decarboxylation of pyruvate (reviewed in Mooney, B P, et al., Annu Rev. Plant Biol. 53:357-375; 2002).

This example is further embodied in cloning of pyruvate dehydrogenase E1α (PDH E1α) genes from microalgae. Degenerate PCR amplification of the Dunaliella or Tetraselmis PDH E1α is desired for homologous gene expression in the chloroplast, which is as or more effective than heterologous expression of Arabidopsis or like genes. This commences with cDNA library construction and screening as described in Example 10.

Primer design can be based on any number of closely related PDH E1α genes by those skilled in the art using for example Arabidopsis GI:2454181; Oryza sativa GI:125547024; or Lyngbya sp. PCC 8106 GI:119492641; Trichodesmium erythraeum GI:113478382; Nodularia spumigena GI:119511804; Synechococcus elongatus PCC 6301 GI:56752159; Porphyra yezoensis GI:90994458; Nostoc sp. PCC 7120 GI:17230200. Degenerate PCR primers are designed based on two conserved motifs of PDH E1α: Motif H: “GKMFGFVH” (SEQ ID NO: 54) and motif P: “EGIPVATGAAF” (SEQ ID NO: 55). Primer Hₛₑₙₛₑ (5′ggiaaratgttyggittygticayi3′) (SEQ ID NO: 56) and Pₐₙₜᵢₛₑₙₛₑ (5′aaigcigciccigtigciaciggiati3′) (SEQ ID NO: 57) are used together with total mRNA template prepared as outlined in example 10 to PCR amplify a product of approximately 291 base pairs. PCR conditions for probe synthesis using Accuprime Pfx DNA Polymerase (Invitrogen) are: initial denaturation at 94° C. for 3 min; four cycles of 94° C. for 15 sec, 52° C. for 30 sec and 72° C. for 45 sec; 10 cycles of 94° C. for 15 sec, 52° C. (decreasing by 1° C. per cycle) for 30 sec, 72° C. for 45 sec; 25 cycles of 94° C. for 15 sec, 42° C. for 30 sec, and 72° C. for 45 sec (increasing by 3 sec per cycle); final extension step of 72° C. for 6 min. The PCR products are labeled and algae cDNA library membranes are hybridized using the North2South Kit (Pierce). Positive clones are identified by hybridization, amplified, and sequenced for identification of the hybridizing DNA insert. Library screening and sequencing continues until the 5′ and 3′ ends of the mRNA have been identified and a full-length clone is obtained. Using this general strategy, additional Dunaliella and Tetraselmis vectors may be generated based on the sequence database obtained from Examples 1 and 2.

### EXAMPLE 13

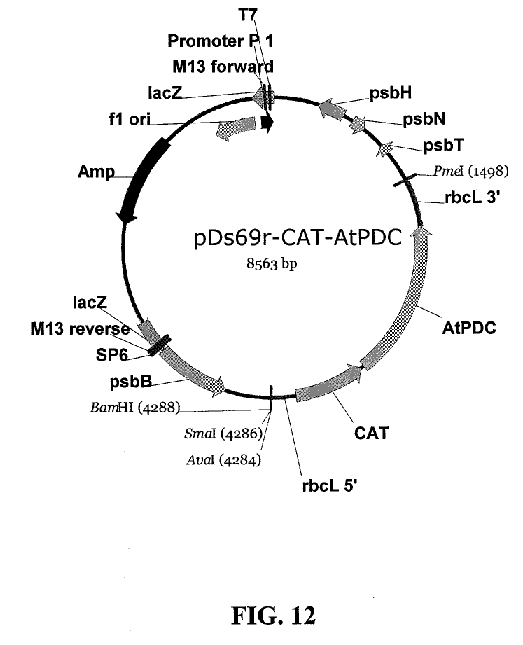

This example embodies targeted integration segments for the chloroplast in which the nucleic acid encodes a gene that participates in fatty acid biosynthesis via conversion of pyruvate into acetyl-coA using pyruvate decarboxylase. Primers 5′ctttatagagtcgactgtgattcaacaatggcggtttc 3′ (SEQ ID NO: 81) and 5′gaaagtcgacttataaggtcaaactatctggattc 3′ (SEQ ID NO: 82) are used to amplify a cDNA encoding Pyruvate Decarboxylase from Arabidopsis thaliana cDNA. Amplification is performed with a Pfx proofreading enzyme using the following conditions; 95° C. 3 min, (94° C. 30 sec, 58° C. 60 sec, 72° C. 40 sec) for 25 cycles, 72° C. 7 min. The 1480 base pair product is digested with SalI and ligated into the XhoI site of pDs69r-CAT, producing vector “pDs69r-CAT-AtPDC” (FIG. 12). Using this general strategy, additional Dunaliella and Tetraselmis vectors may be generated based on the sequence database obtained from Examples 1 and 2.

Following is the sequence of Arabidopsis thaliana LTA2 (plastid E2 subunit of Pyruvate decarboxylase); dihydrolipoyllysine-residue acetyltransferase (LTA2) mRNA (accession NM_{—}113489):

(SEQ ID NO: 58)

aacctcgtct tctccgtcca cttcactctc tctaaactct
ctctcagatc tctctctctc tgtgattcaa caatggcggt
ttcttcttct tcgtttctat cgacagcttc actaaccaat
tccaaatcca acatttcatt cgcttcctca gtatccccat
ccctccgcag cgtcgttttc cgctccacga ctccggcgac
ttctcaccgt cgttcaatga cggtccgatc taagattcgt
gaaattttca tgccggcgtt atcatcaacc atgacggaag
gcaaaatcgt gtcatggatc aaaacagaag gcgagaaact
cgccaaggga gagagtgttg tggttgttga atctgataaa
gccgatatgg atgtagaaac gttttacgat ggttatcttg
ctgcgattgt cgtcggagaa ggtgaaacag ctccggttgg
tgctgcgatt ggattgttag ctgagactga agctgagatc
gaagaagcta agagtaaagc cgcttcgaaa tcttcttctt
ctgtggctga ggctgtcgtt ccatctcctc ctccggttac
ttcttctcct gctccggcga ttgctcaacc ggctccggtg
acggcagtat cagatggtcc gaggaagact gttgcgacgc
cgtatgctaa gaagcttgct aaacaacaca aggttgatat
tgaatccgtt gctggaactg gaccattcgg taggattacg
gcttctgatg tggagacggc ggctggaatt gctccgtcca
aatcctccat cgcaccaccg cctcctcctc cacctccggt
gacggctaaa gcaaccacca ctaatttgcc tcctctgtta
cctgattcaa gcattgttcc tttcacagca atgcaatctg
cagtatctaa gaacatgatt gagagtctct ctgttcctac
attccgtgtt ggttatcctg tgaacactga cgctcttgat
gcactttacg agaaggtgaa gccaaagggt gtaacaatga
cagctttatt agctaaagct gcagggatgg ccttggctca
gcatcctgtg gtgaacgcta gctgcaaaga cgggaagagt
tttagttaca atagtagcat taacattgca gtggcggttg
ctatcaatgg tggcctgatt acgcctgttc tacaagatgc
agataagttg gatttgtact tgttatctca aaaatggaaa
gagctggtgg ggaaagctag aagcaagcaa cttcaacccc
atgaatacaa ctctggaact tttactttat cgaatctcgg
tatgtttgga gtggatagat ttgacgctat tcttccgcca
ggacagggtg ctattatggc tgttggagcg tcaaagccaa
ctgtagttgc tgataaggat ggattcttca gtgtaaaaaa
cacaatgctg gtgaatgtga ctgcagatca tcgcattgtg
tatggagctg acttggctgc ttttctccaa acctttgcaa
agatcattga gaatccagat agtttgacct tataagacgc
caagcgaaga cgagaagtca aaaacagttt ccaaaattcc
tgagccaaat ttttcccaag taaatttttt aatcttcatt
gttcttggtc ttgctctact tcttttgcat ctttttcttc
acttgtgttg tatctgtatt tttgttttca agaatcatca
ttttgggttt taaacaaata atttcctatc cagaatc

### EXAMPLE 14

Use of vectors containing antibiotic-resistance genes as described in the Examples allow growth of algae on various antibiotics of varying concentrations as one means for monitoring nucleic acid introduction into host species of interest. This may also be used for gene-function analysis, for monitoring other payload introduction in trans or unlinked to the antibiotic-resistance genes, but is not limited to these applications. Cells are grown in moderate light (80 E/m²/sec) to a log-phase density of 1×10⁶ cells/mL in appropriate seawater medium for plating. Transgenic antibiotic- or herbicide-resistant colonies appear dark green; the negative control is colorless and growth-inhibited after 21 days, preferably after 12 days, and more preferably after 10 days on liquid or solidified medium. Resistant colonies are re-cultured on selective medium for one or more months to obtain homoplasmy and are maintained under the same or other conditions. Cell growth monitored in liquid culture employs culture tubes, horizontal culture flasks or multi-well culture plates.

A screening process for transgenic Dunaliella is described using plating methods as in the below Examples. For chloramphenicol selection of D. salina using liquid medium, cells at plating densities of 0.5 to 1×10⁶ cells/mL are inhibited by Day 10 in 200 ug/mL chloramphenicol and greater, based on counts of viable cells. Plating densities of 1.9×10⁶ cells/mL are inhibited by Day 10 in 600 ug/mL chloramphenicol and greater, and by 500 ug/mL chloramphenicol and greater by Day 14. Recommended levels for selection when plated on solidified medium at 2×10⁵ cells per 6-cm dish with 0.1% top agar is 700 ug/mL chloramphenicol for both D. salina and D. tertiolecta. For cells that have been subject to electroporation, 600 ug/mL chloramphenicol is the kill point for D. salina plated at 8×10⁵ cells per 6-cm dish.

Dunaliella is very sensitive to the herbicide gluphosinate as selection agent in liquid medium based on replicated platings at 1×10⁶ cells/mL. Concentrations of 5 ug/mL gluphosinate and greater inhibit cell growth of D. salina almost immediately. D. tertiolecta shows inhibition of cell growth by Day 14 from 2 ug/mL gluphosinate and greater. Recommended levels for selection when plated on solidified medium at 2×10⁵ cells per 6-cm dish with 0.1% top agar is 14 ug/mL and 16 ug/mL gluphosinate for D. salina and D. tertiolecta, respectively.

A screening process for transgenic Tetraselmis is described based on replicated platings. Log phase cultures are concentrated by centrifugation of 700 mL at 2844×g to achieve 8×10⁶ cells/mL when resuspended in 35 mL or similar of culture medium. Media are either 100% ASW modified by using F/2 vitamins (see website at http://cmmed.hawaii.edu/research/HICC/pages/golden/Media/ASW_Media.htm, modified from Brown L. Phycologia 21: 408-410; 1982), or F/2 35 psu-Si media (Guillard, R. R. L. and Ryther, J. H. Can. J. Microbiol. 8: 229-239; 1962). Both media are at 35 psu for 3.5% NaCl. For preparation of medium solidified with 0.75% agar, 4.5 g of Difco Bacto Agar is autoclaved in 1 L bottles. To this is added 600 mL of sterile media, which is heated until the agar goes into solution. 10 mL of agar with calculated amounts of antibiotics are used in 6 cm culture dishes. A 0.2% top agar for plating of algae cells is prepared by adding 0.5 g of Difco Bacto Agar to 250 mL of either 100% ASW and F/2 35 psu-Si media. The agar is used at 38° C. for plating of cells in a 1:1 top-agar: concentrated cells mix, with generally 1 mL per plate. Cultures are incubated at room temperature (20° C.-30° C. avg. 25° C.), 22 uM/m²sec light intensity with a photoperiod of 14 hr days/10 hr nights. Liquid cultures are further exemplified by use of 5 mL of concentrated culture mixed with calculated amounts of antibiotic in test tubes, with incubation in vertical racks at room temperature (20° C.-30° C. avg. 25° C.), 22 uM/m²sec light intensity with a photoperiod of 14 hours. Growth is assessed visually at Day 10.

Results on solidified medium show that less than 100 mg/L chloramphenicol is required to inhibit Tetraselmis at this plating density in either 100% ASW or F/2 35 psu-Si media. Further, greater than 1000 mg/L kanamycin is required and thus this antibiotic is undesirable for Tetraselmis at typical plating densities. The herbicide gluphosinate is toxic to Tetraselmis at 15 mg/L by Day 7, but re-growth is observed by Day 15 and thus is not preferred as selection agent in solidified medium. For liquid medium, results from hemocytometer counts of viable cells show that Tetraselmis cells undergo three divisions in 7 days in both media at these culture conditions. In contrast, during Day 0 to Day 7, cells in 2.5 mg/L up to 20 mg/L gluphosinate show a decrease in viability from 31% up to 60% in F/2, and 52% up to 84% in 100% ASW medium, respectively. During Day 7 to Day 15, cells in 100% ASW undergo a first doubling in 2.5, 5.0 and 10.0 mg/L gluphosinate, but remain inhibited in 15 and 20 mg/L gluphosinate. By Day 21, cell density has almost doubled in 15 mg/L gluphosinate, but not at 20 mg/L gluphosinate, suggesting that both 15 and 20 mg/L gluphosinate can be used for two-week selection, and that 20 mg/L gluphosinate should be used for three-week selection in 100% ASW. During Day 7 to Day 15 in F/2 liquid medium, cell death is at 87% and 91% at 15 and 20 mg/L gluphosinate, respectively. Some re-growth to initial inoculum levels is seen by Day 21 in 15 mg/L gluphosinate in F/2 liquid, but complete death results by Day 21 in 20 mg/L gluphosinate, suggesting that both 15 and 20 mg/L gluphosinate can be used for two-week selection in F/2 liquid, and that 20 mg/L gluphosinate should be used for three-week selection in F/2 medium. Using this general strategy, additional Dunaliella and Tetraselmis vectors may be generated based on the sequence database obtained from Examples 1 and 2.

### EXAMPLE 15

This example illustrates one possible method for plastid transformation.

Nucleic acid uptake by eukaryotic microalgae is by using one of any such methods as electroporation, magnetophoresis, and particle inflow gun. This specific example describes a preferred method of transformation by electroporation for Dunaliella and Tetraselmis using chloroplast expression vector pDs69r-CAT-IPPI, and can be adapted for other algae, vectors, and selection agents by those skilled in the art. The protocol is not limited to uptake of nucleic acids, as other payload such as quantum dots are also shown to be internalized by the cells following treatment.

Cells of Dunaliella are grown in 0.1 M NaCl or 1.0 M NaCl Melis medium, with 0.025 M NaHCO₃, 0.2 M Tris/Hcl pH 7.4, 0.1 M KNO₃, 0.1 M MgCl₂.6H₂O, 0.1 M MgSO₄.7H₂O, 6 mM CaCl2.6H₂O, 2 mM K₂HPO₄, and 0.04 mM FeCl₃.6H₂O in 0.4 mM EDTA, to a cell density of 1-4×10⁶ cells/mL and adjusted preferably to a density of 1-3×10⁶ cells/mL. Cells of Tetraselmis spp. Are grown in 100% ASW. Approximately 388 uL of the cells per 0.4 cm parallel-plate cuvette are used for each electroporation treatment. Cells, spun down in a 1.5 ml microcentrifuge tube for 4 min at 14,000 rpm or until a pellet forms to enable removal of the supernatant, are resuspended immediately in electroporation buffer consisting of algae culture medium amended with 40 mM sucrose. Transforming plasmid DNA (4-10 ug, preferably the latter), previously linearized by an appropriate enzyme such as pml1 or nde1 for vector pDs69r-CAT-IPPI, are added along with denatured salmon sperm carrier DNA, (80 ug from 11 mg/mL stock, Sigma-Aldrich), per cuvette. A typical reaction mixture includes 388 uL cells, 4.4 uL DNA, 7.3 uL carrier DNA for a 400 uL total reaction volume. The mixture is transferred to a cuvette for placement on ice for 5 min prior to electroporation. Treatment settings using a BioRad Genepulser Xcell electroporator range from 72, 297, 196 and 396 V at 50 microFaraday, 100 Ohm and 6.9 msec. Negative controls consist of cells in buffer with nucleic acids that receive no electroporation or cells that are electroporated in the absence of payload.

Following electroporation, the contents of each cuvette are plated, with 200 ul of cell suspension plated onto 1.5% agar-solidified medium comprised of 0.1 Melis or 1.0 M Melis medium, as above, in 6-cm plastic Petri dishes, and the remaining 200 uL spread over a selection plate of algae medium amended with 600 ug/mL chloramphenicol. Alternatively, a warmed (38° C.) 0.2% top-agar in algae medium can be used for ease of plating using a 1:1 dilution with cells for 400 uL total per plate. This ensures uniform spreading of the cells on the plate. Plates are dried under low light (<10 umol/m²sec) before wrapping with Parafilm and moved under higher light (50-100 umol/m²sec, preferably 50-60 umol/m²sec). Dunaliella may be left in electroporation buffer for 60 hr at room temperature prior to plating with no noticeable affect on cell appearance or motility. In another manifestation, the contents of each cuvette are cultured in liquid medium rather than on solidified medium. Samples treated under the same parameters are collected in well of a 24-well plate, diluted 1:1 with algae growth medium for total volume of 800 uL. These are placed under 50 umol/m²sec for 2 days. Then enough chloramphenicol added for a concentration of 500-800 ug/mL per selection well, and more preferably of 600 ug/mL chloramphenicol for the initial cell density employed.

Quantum dots (Q-dots) are used for visualization of intracellular payload in target cells following electroporation. Such algal cells are detected by flow cytometry (FCM) based on their unique fluorescent emission spectra. Use of Quantum dots (Q-dots) to monitor cellular uptake and trafficking of plasmid DNA is accomplished by binding the Q-dots (525 nm) to plasmid DNA. The pGeneGrip™ Biotin/Blank vector, purchased from Genlantis (San Diego, Calif.), arrives irreversibly-labeled with a peptide nucleic acid (PNA) linker that is attached to an AGAGAGAG binding site on the plasmid. The free end of the PNA linker is covalently labeled with biotin. The biotin-labeled plasmid DNA readily binds molecules linked to streptavidin. Q-dots are purchased as a strepavidin conjugate (Molecular Probes/Invitrogen). Plasmid DNA-biotin (10 ug, ˜30 picomoles) is conjugated overnight at room temperature with 16.67 ul of Q-dots:streptavidin (˜167 picomoles of streptavidin, giving a 1:10 molar ratio of plasmid DNA to Q-dots). After the incubation, the mixture is passed over a sephacryl-500-HR column to remove the free Q-dots:streptavidin. Removal of free Q-dots is confirmed by gel electrophoresis. 3 ug of DNA/quantum dots is subjected to electrophoresis in a 0.8% agarose TAE gel. The fluorescently-labeled molecules are visualized using a UV transilluminator. A predominant band (Band 1) with slower mobility than the Q-dots alone (Band 2) corresponds to the bulk of the DNA-conjugated Q-dots.

Electroporation of cells at a density of 3-4×10⁶ cells/mL is carried out using 396 V at 50 microFaraday, 100 Ohm and 6.9 msec. Five replicates of each treatment are performed and then pooled together in one tube. Cells of all treatments were incubated for 3 hr prior to analysis by flow cytometry. Up to six different controls are included: 1) Cells with Q-dots plus DNA but not electroporated; 2) Cells plus electroporation buffer that are electroporated (no Q-dots+DNA); 3) Cells plus electroporation buffer, untreated); 4) Electroporation buffer alone, electroporated; 5) Electroporation buffer alone, untreated; and 6) Q-dots plus DNA in electroporation buffer, untreated.

Enrichment of Dunaliella cells containing DNA-conjugated quantum dots is performed using a laser flow cytometer. Samples are sorted on a Beckman-Coulter Altra flow cytometer equipped with multiple lasers, including a water-cooled 488 nm argon ion laser. The instrument has several detectors, including those optimized for chlorophyll (680 nm bandpass filter) and GFP (525 nm bandpass filter). Populations can be sorted will be distinguished based on their light scatter (forward and 90 degree), chlorophyll and GFP or similar fluorescence, as appropriate; enrichment of Q-dot-treated Dunaliella cells follows sorting using a 525 nm bandpass filter. Those cells containing the DNA-conjugated Q-dots sort into window “B” compared to all other cells sorted into window “A”. The flow cytometer is capable of sorting two populations into separate receptacles simultaneously, with a typical sort purity of >98%. Further, this technique is used for selecting Dunaliella cells with altered isoprenoid flux affecting total chlorophyll, with the 680 nm filter, resulting from transgene expression of IPPI.

Results show that 2.1% of total cells electroporated with conjugated Q-dots contain the fluorescent marker; such results are confirmed in a separate experiment which show 5.3% of total cells sorted with 525 nm fluorescence expected for cells containing Q-dots. All the negative controls give the expected results of either zero, minimal or possible artifactual passive uptake. Cells incubated with conjugated Q-dots in the absence of electroporation show 0% or 0.2% cells sorted into the fluorescent cell window, similar to the 0% cells in buffer alone. Tetraselmis algae cells can also be sorted at 525 nm, with no background interfering fluorescence.

Algae cells containing inserted nucleic acid payload can be enriched and cultured following flow cytometry. Cells cultured after treatment and sorting by flow cytometry are free of contamination, proliferate, and can be increased in volume as with any other cell culture as is known in the art. Cells can be preserved with paraformaldehyde, to stop motion of flagellated cells, and observed under the light microscope. No significant differences in cell appearance are observed between the electroporated samples and the controls, confirming that electroporation of cells followed by flow cytometry will yield live, non-compromised cells for subsequent plating experiments.

Cells treated by electroporation are examined fluorimetrically two days after treatment for transient expression of reporter gene fluorescence compared to controls receiving no transgenesis treatment. Expression of beta-glucuronidase enzyme in Dunaliella follows four different electroporation treatments, using a BioRad GenePulser Xcell electroporator range from 72, 297, 196 and 396 V at 50 microFaraday, 100 Ohm and 6.9 msec, using linearized nuclear expression vector pBI426 with the Cauliflower Mosaic Virus 35S promoter. Expression is measured as absolute fluorescence per microgram protein per microliter sample over time using the 4-MUG assay (R A Jefferson, Assaying chimeric genes in plants: The GUS gene fusion system, Plant Molecular Biology Reporter 5: 387-405; 1987) using the MGT GUS Reporter Activity Detection Kit (Marker Gene Technologies, Eugene Oreg., #M0877) with a Titertek Fluoroskan fluorimeter in 96-well flat-bottomed microtitre plates. There is a detection level of 1 pmol 4-methylumbelliferone up to 6000 pmol per well, with a performance range of excitation wavelength 330-380 nm and emission wavelength 430-530 nm. Fluorescence increases over 90 min for all four electroporation conditions but remains zero for the negative control among four replicate wells for each treatment.

Further, Dunaliella and Tetraselmis cells are conferred stable resistance to chloramphenicol by electroporation treatment with pmlI-linearized chloroplast vector pDs69r-CAT-IPPI. Electroporation of cells, at a density of 2×10⁶ cells/mL in 1 M NaCl Melis medium and pre-chilled for 5 min, is carried out using 396 V at 50 microFaraday, 100 Ohm and 6.9 msec, and cells from each cuvette are plated in a well of a 24-well plate diluted with 400 ul of fresh growth medium. Selection commences on Day 3 using 5 different concentrations of selection agent, namely 0, 500, 600, 700, 800 ug/mL chloramphenicol for a total of 0.8 mL in each well, with two to four replicates of each plating concentration. Cells are cultured under 50-60 umol/m²sec, in a 14 hr day/10 hr night at a temperature range preferably of 23° C. to 28° C. Sensitivity to the antibiotic is seen as a yellowing-bleaching of the cells and change in motility for both Dunaliella and Tetraselmis when viewed under 400× using an Olympus 1X71 inverted epifluorescent microscope.

At Day 4, about 50% of the cells plated in 600 ug/mL chloramphenicol after electroporation without DNA (negative controls) are green and moving in circles rather than the more common directional swimming. About 20% of the cells plated in 600 ug/mL chloramphenicol after electroporation with DNA are green, with some moving directionally as opposed to spinning in circles. Cells in liquid medium without antibiotic (positive controls) are predominantly green and moving directionally or are settled on the bottom of the plate and immobile. On Day 12, cells not settled on the well bottom are subcultured into new plates with an addition of equal volume of fresh medium+/−antibiotic per well. Cells that have adhered to the wells are incubated in fresh medium in the existing wells. By Day 13, all negative control cells are bleached and immobile in all levels of antibiotic. Positive control cells are green and motile; those settled on well surfaces remain green but are largely immobile. Cells treated with pDs69r-CAT-IPPI and plated in chloramphenicol show some green cells that are moving both directionally or in circular motion, even in 700 and 800 ug/mL chloramphenicol. By Day 22, all negative control cells remain bleached and immobile; positive control cells remain predominantly green and motile; and a number of cells treated with DNA are identified as being transformed based on being green, motile (documented by video), and in some cases being rounded with the appearance of imminent division. Replicated experiments illustrate that about 8% of the cells plated in 600 ug/mL chloramphenicol after electroporation with DNA are green at Day 10, whereas all controls in 600 ug/mL chloramphenicol are completely bleached. The chloramphenicol-resistant cells retain motility, with slow directional or spinning motion unless settled on the well bottoms. Wells with 700 ug/mL chloramphenicol have fewer green cells, approximated at 3%, and show slow motion in place. Upon transfer to fresh medium, green cells recover directional motion whereas all negative control cells remain bleached and immobile.

Similar results are observed after two weeks when cells are treated with electroporation conditions of 297, 196 or 396 V at 50 microFaraday, 100 Ohm and 6.9 msec, and plated only in 0 or 600 ug/mL chloramphenicol; all replicates of the negative controls in antibiotic are bleached, positive controls are green, and DNA-treated cells have some green, motile algae present. Based on this vector and method, cultures are pooled and enriched for stably transformed cells at Day 12 using flow cytometry with a 680 nm bandpass filter for chlorophyll fluorescence detection, and grown out under diminishing antibiotic concentrations with weekly dilution by 100 uL growth medium lacking chloramphenicol. Alternatively, cultures are supplemented weekly with fresh medium with or without antibiotic for an additional 14-21 days prior to bulking in flask culture.

### EXAMPLE 16

This example illustrates one possible method of genetic transformation with such vectors as described in the Examples using a converging magnetic field for moving pole magnetophoresis. The magnetophoresis reaction mixture is prepared beginning with linear magnetizable particles of 100 nm tips, tapered or serpentine in configuration, with any combination of lengths such as, but not limited to 10, 25, 50, 100, or 500 um, comprised of a nickel-cobalt core and optional glass-coated surface, suspended in approximately 100 uL of growth medium in 1.5 mL microcentrifuge tubes, the volume being adjusted downward to account for any extra volume needed if using dilute vector DNA stock. To this is added 500 uL algae cells, such as Dunaliella cells, concentrated by centrifugation to reach a cell density of 2-4×10̂8 cells/mL in algae medium such as 0.1 M or 1.0 M NaCl Melis medium as determined by hemacytometer counting; the algae cell volume is adjusted as necessary to meet the total volume. Denatured salmon sperm carrier DNA (7.5 uL from 11 mg/mL stock, Sigma-Aldrich; previously boiled for 5 min), and linearized transforming vector (8 to 20 ug from a 1 mg/mL preparation) are added next. Finally 75 uL of 42% polyethylene glycol (PEG) are added immediately before treatment and mixed by inversion. The filter-sterilized PEG stock consists of 21 g of 8000 MW PEG dissolved in 50 mL water to yield a 42% solution. Total reaction volume is 690 uL.

For moving pole magnetophoresis for microalgae treatment, the microcentrifuge tube containing the reaction mixture is positioned centrally and in direct contact on a Corning Stirrer/Hot Plate set at full stir speed (setting 10) and heat at between 39° to 42° C. (setting between 2 and 3), preferably at 42° C. A 2-inch×¼-inch neodymium cylindrical magnet, suspended above the reaction mixture by a clamp stand, maintains dispersal of the nanomagnets. After 2.5 min of treatment the mixture is transferred to a sterile container that holds at least 6-10 mL, such as a 15 mL centrifuge tube. A dilution is made by adding 1.82 mL of algae culture medium to the mixture, to allow a preferred plating density. To this is added 2.5 ml of dissolved top-agar (autoclaved 0.2% agar in algae medium such as 0.1 M NaCl Melis) at 38° C. (1:1 dilution). Mix and plate 500 uL of solution per 6-cm plate containing algae medium such as 0.1 M NaCl Melis medium prepared with and without selection agent for selection of transformants under cell survival densities. Allow plates to dry for 2-3 days under low light (<10 umol/m²sec). When dry, plates are wrapped in Parafilm and cultured under higher light of 85-100 umol/m²-sec. Plates are observed for colony growth beginning at day 10 and ending no later than day 21, depending on the antibiotic, after which colonies are photographed and subcultured to fresh selection medium.

Typical data are exemplified by dark green colonies of Dunaliella salina formed on medium containing 0.5 M phleomycin in replicated plates 3 weeks after magnetophoresis treatment of 2.5 min with linearized Chlamydomonas nuclear expression vector pMFgfpble using 25-micron tapered nanomagnets. Controls treated in the absence of DNA are unable to grow on 0.5 M phleomycin but form multiple colonies on 0.1 M Melis medium lacking antibiotic. Further typical data are exemplified by small dark green colonies of Dunaliella salina formed on medium containing 100 ug/mL chloramphenicol 12 days after magnetophoresis treatment with linearized Dunaliella chloroplast expression vector pDs69r-CAT-IPPI. This level of antibiotic gives 100% kill of cells after treatment by magnetophoresis in the absence of transforming DNA, as the final plating density of remaining viable cells is lower than the initial treatment density of viable cells. At Day 12 these colonies are subcultured to a fresh plate of medium containing 100 ug/mL chloramphenicol. By Day 23 the resistant colonies continue to grow while all negative controls on replicated selection plates are already non-viable by Day 12. Using this general strategy, additional Dunaliella and Tetraselmis transformants may be generated.

### EXAMPLE 17

This example describes one possible method of introduction of nucleic acids into target algae by particle inflow gun bombardment. These conditions introduce nucleic acids representative of oligonucleotides into target algae, including but not limited to plasmid DNA sequences intended for transformation. Microparticle bombardment employs a Particle Inflow Gun (PIG) fabricated by Kiwi Scientific (Levin, New Zealand).

Cells in log phase culture are counted using a hemacytometer, centrifuged for 5-10 min at 1000 rpm, and resuspended in fresh liquid medium for a cell density of 1.7×10̂8 cells/ml. From this suspension 0.6 ml will be applied to each 10-cm plate solidified with 1.2% Bacto Agar. To allow cells a recovery period before antibiotic selection is applied, some plates use nylon filters overlaid on the agar; for direct selection no filters are used. Plates placed 10 cm from the opening of the Swinnex filter (SX0001300, Millipore, Bedford Mass.) are treated at 70 psi with a helium blast of 20 milliseconds with the chamber vacuum gauge reading −12.5 psi at the time of blast. These PIG parameters were optimized for depth penetration and lateral particle distributions using dark field microscope and automated image processing analyses courtesy of Seashell Technologies (La Jolla, Calif.). Preferred conditions result in 60-70% of the particles penetrating to a depth of between 6-20 microns. Transforming DNA is precipitated onto S550d DNAdel™ (550 nm diameter) gold carrier particles using the protocol recommended by the manufacturer (Seashell Technology, La Jolla, Calif.), with 60 ug particles and 0.24 ug DNA delivered per shot. Three shots are made per plate, targeted to different regions of cells. After shooting, plates are sealed with Parafilm and placed at ambient low light of 10 uM/m²-sec or less for two days. On Day 3, the cells on nylon filters are transferred to Petri dishes or rinsed and cultured in liquid medium in multiwell plates with any desired selection medium. Using this general strategy, additional Dunaliella and Tetraselmis transformants may be generated.

### EXAMPLE 18

This example illustrates one possible method for genetic transformation of other target algae with such vectors as described in the Examples by electroporation of Chlorella species. Chlorella may be fresh water or salt water species; some are naturally robust and can proliferate in under both fresh and saline conditions. Yet other Chlorella can be adapated or mutagenized to grow become salt-tolerant or fresh water-tolerant. Examples of species includes but is not limited to C. ellipsoidea, C. luteoviridis, C. miniata, C. protothecoides, C. pyrenoidosa, C. saccharophilia, C. sorokiniana, C. variegata, C. vulgaris, C. xanthella, and C. zopfingiensis. A Chlorella strain that can be cultivated under heterotrophic conditions, wherein an organic carbon source is supplied is preferable in some production systems as is known in the art. For example Chlorella are known to be produced at large scale for fishery feeds and nutritional supplements under a combination of dark heterotrophic and illuminated heterotrophic or mixotrophic conditions.

Any culture medium can be used wherein the desired strain of Chlorella can proliferate. In one embodiment, cells of target algae are grown in YA medium, to a cell density of 1-4×10⁶ cells/mL. In another embodiment, this medium can be supplemented with 1% by weight of sodium chloride. In yet another embodiment, the culture medium is supplemented with glucose and has the overall composition per 1 L of 3 g Difco yeast extract, g Bactopeptone, 5 g malt extract, and 10 g glucose, with 20 g agar for solidified media.

Cells are collected by centrifugation at room temperature at 500×g, washed with HS medium and adjusted preferably to a density of 1-3×10⁸ cells/mL by resuspending in sterile distilled water. 80 to 100 microliters of cells are transferred to a sterile parallel-plate cuvette with 0.2 cm spacing between electrodes. Transforming plasmid DNA, 4-10 ug, preferably 5 ug, is added to the cuvette. A typical reaction mixture includes 100 uL cells, 5 uL DNA, for a 105 uL total reaction volume. The mixture in the cuvette is placed on ice for 5 min prior to electroporation. Treatment settings using a BioRad Genepulser Xcell electroporator range from 600 to 2000 V/cm at 25 microFaraday and 200 Ohm. Negative controls consist of cells in sterile distilled water with nucleic acids that receive no electroporation, or cells that are electroporated in the absence of payload. After electroporation, the Chlorella cells are resuspended in 5 ml of fresh YA (or saline adjusted) medium and allowed to recover for 24 hours at room temperature in the dark.

Typical data are exemplified by dark green colonies of Chlorella formed on YA agar (or saline adjusted) plates containing 50 ug/ml of hygromycin B 10 to 14 days after electroporation treatment with a DNA vector as described in the Examples. Vector DNA contains the hygromycin phosphotransferase gene (hph) of Escherichia coli to provide transformed target algae with resistance to hygromycin. Controls treated in the absence of DNA, or with DNA but not electroporated, are unable to grow on 50 ug/ml of hygromycin B but form multiple colonies on YA agar lacking antibiotic. By about Day 23 the resistant colonies continue to grow while all negative controls on replicated selection plates are already non-viable by Day 14. Using this general strategy, additional Chlorella transformants may be generated.

### EXAMPLE 19

This example illustrates one possible method for conjugation to introduce a nucleic acid vector described in the Examples into target cells such as Cyanobacteria.

The appropriate cyanobacteria strain is grown for 3-5 days in BG11 NO₃+10 mM HEPES pH 8.0+5 mM sodium bicarbonate and any appropriate antibiotic at 25-30° C. under illumination of approximately 50 μmol photons/m2/s in a 12 hour photoperiod until the culture is bright green.

An E. coli strain which contains a mobilizable shuttle vector and a helper plasmid is grown. Transformants are selected on LB agar plates containing ampicillin at 50 ug/ml, chloramphenicol at 10 ug/ml and either streptomycin/spectinomycin at 25 ug/ml each or 50 ug/ml kanamycin. This transformed E. coli is grown overnight in 2 ml TB broth with the same antibiotics as those used for selecting transformants).

Using the 2 ml overnight culture, LB broth is inoculated with the same antibiotic selection to OD₆₀₀˜0.05 and grow to ˜0.7. For example, inoculate 40 ml LB broth with 500 ul of the overnight TB culture and grow for 3 hours. The E. coli are washed 2× with at least 1/10 volume BG11 NO₃ by centrifuging the cells at 5000×g for 5 min, discarding the supernatant, and resuspending the cells in 10 ml BG-11. After the second wash, the cells are centrifuged again and the supernatant is discarded. The E. coli is resuspended in a final volume of BG-11 that corresponds to 1.2 mL per 40 mL starting culture.

If performing conjugation with a replicating plasmid, 1/10 and 1/100 dilutions of the cyanobacteria culture are used. If performing conjugation using a non-replicating plasmid, the cyanobacteria culture also is used in undiluted form. 150 ul of cyanobacteria is mixed with 150 ul of the E. coli and the resulting 300 ul is pipetted directly onto a BG11 NO₃ plate containing 5% LB or onto a filter on a BG11NO₃+5% LB plate. All liquid is absorbed into the plate and then plates are transferred to an incubator and placed upside down covered both top and bottom by a paper towel. The paper towel is removed after 1 day.

After two days, filters are transferred to agar plates containing BG11NO₃ with neomycin or kanamycin 50 mg/L if using the DNA vector pScyAFT-aphA3 as described in the Examples. If a filter is not being used, the cells are resuspended by spreading 0.5 ml of BG-11 liquid onto the plate, the liquid and cells are collected with a pipette, and the cell suspension is spread on agar plates containing BG11NO₃ with appropriate antibiotic selection. Colonies of cyanobacteria appear in about 2 weeks.

After isolating recombinant colonies, if necessary, cells that retain an antibiotic resistance cassette in the chromosome are grown in liquid with selection for 3-5 days, sonicated to fragment filaments to obtain single cells, and then plated on BG11NO₃ agar plates with 5% sucrose and antibiotic selection.

### EXAMPLE 20

This example illustrates one possible method for transformation of target cells of cyanobacteria by uptake of DNA.

The appropriate cyanobacteria strain is grown for 2 days in BG11 NO₃+10 mM HEPES pH 8.0+5 mM sodium bicarbonate, 2 mM EDTA and any appropriate antibiotic at 25-30° C. under illumination of approximately 50 μmol photons/m2/s in a 12 hour photoperiod until the culture is bright green. Using this culture, fresh media of the same is inoculated to OD₇₃₀ 0.05 and grow to OD₇₃₀ 0.8. The cyanobacteria are washed 2× with fresh BG11 medium by centrifuging the cells at 5000×g for 5 min, discarding the supernatant, and resuspending the cells in 10 ml BG-11. After the second wash, the cells are centrifuged again and the supernatant is discarded. The cyanobacteria are resuspended in fresh BG-11 medium to achieve a cell density of 1×10⁹ cells/ml.

Vector DNA as described in the Examples is added to achieve a concentration of 20 μg/ml to 50 μg/ml. The solution is mixed gently and incubated under illumination of approximately 50 μmol photons/m2/s for 5 hours.

The cell suspension is pipetted directly onto a BG11 NO₃ plate or onto a filter on a BG11NO₃ plate. All liquid is absorbed into the plate and then plates are transferred to an incubator and placed upside down covered both top and bottom by a paper towel. The cultures are allowed to recover for 4 to 5 hours.

The filters are transferred to agar plates containing BG11NO₃ with kanamycin 50 mg/L if using a DNA vector such as pScyAFT-aphA3, described elsewhere herein. If a filter is not being used, the cells are resuspended by spreading 0.5 ml of BG-11 liquid onto the plate, the liquid and cells are collected with a pipette, and the cell suspension is spread on agar plates containing BG11NO₃ with appropriate antibiotic selection. Colonies appear in about 2 weeks.

After isolating recombinant colonies, if necessary, cells that retain an antibiotic resistance cassette in the chromosome are grown in liquid with selection for 3-5 days, sonicated to fragment filaments to obtain single cells, and then plated on BG11NO₃ agar plates with 5% sucrose and antibiotic selection.

### EXAMPLE 21

This example illustrates one possible method for genetic transformation of cells by targeting nucleic acid sequences to a conserved Cluster of Orthologous Groups (COG). Standard modern molecular biology techniques for manipulating nucleic acid sequences in vitro are combined with in vivo propagation of the sequences in the host cell of choice. Hybrid plasmid vectors are constructed to shuttle nucleic acid sequences between the propagation host cell, preferably an Escherichia coli cell, and the expression host cell, preferably a cyanobacteria. In this example, the host cell for integration and expression of the desired nucleic acid molecule is a prokaryote, preferably a cyanobacteria.

The hybrid vectors contain sequences that allow replication of the plasmid in Escherichia coli and nucleic acid sequences that are derived from the genome of the cyanobacteria, and additional nucleic acid sequences of interest such as those described in the Examples. A number two ranked cyanobacterial cluster of orthologous groups, which contains mostly genes for lipid and amino acid metabolism, facilitates expression of the nucleic acid sequences from the Examples at a level that is well tolerated by the host cell metabolism and appropriate to achieve the desired modifications of carbon metabolism, for example, isoprenoid and fatty acid biosynthesis.

### EXAMPLE 22

This example illustrates one possible method for genetic manipulation of cyanobacteria host cells by targeting nucleic acid sequences to a conserved Cluster of Orthologous Groups (COG). General features of nucleic acid sequences promoting homologous recombination into the target locus of the chromosome of the expression host cell are as described in the Background of the Invention—Vectors. More specific features are described here.

This example illustrates one possible method for preparation of backbone vectors for targeted integration of DNA segments into the genome of prokaryotes, preferably cyanobacteria.

Backbone vectors are desired for targeted integration of DNA segments in the cyanobacteria genome. In one embodiment of this example, genomic DNA sequences of Synechocystis sp. PCC6803 (GenBank accession number BA000022) are used to produce vector pScyAFT. PCR primers: Forward 5′ ctataccGAATTC cgaaaccttgctctcactag 3′ (SEQ ID NO: 68) and Reverse 5′ ccgtataTCTAGAgggcgattaatttacccaaac 3′ (SEQ ID NO: 69) are used to amplify a 4080 base pair fragment of the Synechocystis genomic DNA from nucleotides 819421 through 823500. This region of the genome includes coding sequences for the Acp, Fab, and Tkt genes, corresponding to CyOGs 00915, 00914 and 00913, respectively. This 4106 base pair PCR product has a unique EcoRI site added by primer Forward and a unique XbaI site added by primer Reverse to enable directional cloning of the fragment into the general purpose cloning vector pUC19 (ATCC accession number 37254) after digestion of both molecules with the restriction enzymes.

Below is the PCR product of primers Forward and Reverse with genomic DNA from Synechocystis sp PCC6803 as a template:

(SEQ ID NO: 70)

5′ctataccGAATTCcgaaaccttgctctcactaggaatgcccctgggca
acggattaccagccgcaacagtggcccaagcctatgttcatagcttagaa
ggcactatgacaggagaagtgctctatccgtagtaaccatatcttggttt
actcttcccccatcatggattggagataattttccagtccagaattactg
ataagccattgctgggactctaaccagtcaatttgttcttctgtttcttc
aagaatttccgacaacacatcccggcttacatagtcccgttgggtttcaa
agaaggcaatgctgttaactaaaccatccctaatgccttggttcatggtc
agatcattgcccaggatttccggtaccgtctcgccgatgagaagtttttc
caaattttggagattggggagtccttccaaaaataaaacccgctcgatca
ggctatcggcctgcttcattgccttgatggatactttatattcgtactga
ttaagtgcgttcagcccccaatttttgcacatgcgagcatggagaaaata
ttggttaatcgcagtaagttgtagctttaacgcttggttgagatgttgtc
tgacttccaggttgccttccatgttgttatcctctgatgtggagttttgt
ttgatgttgttgtttccatttttacccattcacggtccgacgacggagtt
atttactgggacagcaataaattgtttaaattgttttaatgttttacccc
tgggaaaattgcctttttctcaaaggaagtgtccctctctgaccttaaac
tgaaccaatatggctgatttgtttgtcggtgccccagttcgtttaattgc
ccgtcccccctatttgaaaaccgctgatcccatgcccatgctccgtcctc
cggatttattggcgatcgccgcggagggaatggtggtagaccgtcgaccg
gctggctattggggagtaaagtttgaccgaggcacttttctgttggaaag
ccagtatttggaagtgattcggcctcaggaagaaaaaacggaagtctcgg
attaagaacgccgagtaaatgaccaagtttaatctaaaaatatggcatca
actgtaaatcgcctttttttagcaattttgaccatagccagcttcagcct
tagtggaggttatggatatgttcccgttcccatggcgatcgccgctgacg
tcccagaactgacagcaaaggtgcccaattatttggataaaatccaattt
cctctaggggttatcgatgtctatggattgatgggcccagaggatggtaa
acgttcccaaggctatgaattttgtgttgtgcccgagaaaaaaagtgaag
ttttggccatcgatccctcactcacattttcgtctagccctggtcgcatc
ggttgcccccaggaacaattactgtgcctaggagatacccagcaaccaaa
ttggcaggccattctctttgccctggcccggttgagttacatagaaaaaa
tcttgccccactggggagaatagaagcccctatttgacaaatgtttctgg
ccaagggacaggggaagcatctagtgcaagggatacctttccgttaagat
ggttaacgctgaacaattgagcgcattgctaaccaggcggccctgcgaca
gccccaagctgtcccccgttttgctggcgatcggccgttgacccagcacg
aaaactcttcttttatagttaaaggtattgtaatgaatcaggaaattttt
gaaaaagtaaaaaaaatcgtcgtggaacagttggaagtggatcctgacaa
agtgacccccgatgccacctttgccgaagatttaggggctgattccctcg
atacagtggaattggtcatggccctggaagaagagtttgatattgaaatt
cccgatgaagtggcggaaaccattgataccgtgggcaaagccgttgagca
tatcgaaagtaaataaattccggccatagccccgactccccccatagatc
tttggagccgagttctcggacggtttaagccactgtttaggactgcccca
atgccggttttgggtttatcagtttgcccctcgggctaggccctggcccc
gtcgctgtatctttgcggagaactccaggggagtcccctccccgattcta
tctattaagtaccatggcaaatttggaaaagaaacgtgttgttgtaacgg
gattgggagccatcacccccatcggtaatactctccaagactattggcaa
ggcttaatggagggtcgtaacggcattggccccattacccgtttcgatgc
tagtgaccaagcctgccgttttggaggggaagtaaaggattttgatgcta
cccagtttcttgaccgcaaagaagctaaacggatggaccggttttgccat
tttgctgtttgtgccagtcaacaggcaattaacgatgctaagttggtgat
taacgaactcaatgccgatgaaatcggggtattgattggcacgggcattg
gtggtttgaaagtactggaagatcaacaaaccattctgttggataagggt
cctagccgttgcagtccttttatgatcccgatgatgatcgccaacatggc
ctctgggttaaccgccatcaacttaggggccaagggtcccaataactgta
cggtgacggcctgtgcggcgggttccaatgccattggagatgcgtttcgt
ttggtgcaaaatggctatgctaaggcaatgatttgcggtggcacggaagc
ggccattaccccgctgagctatgcaggttttgcttcggcccgggctttat
ctttccgcaatgatgatcccctccatgccagtcgtcccttcgataaggac
cgggatggttttgtgatgggggaaggatcgggcattttgatcctagaaga
attggaatccgccttggcccggggagcaaaaatttatggggaaatggtgg
gctatgccatgacctgtgatgcctatcacattaccgccccagtgccggat
ggtcggggagccaccagggcgatcgcctgggccttaaaagacagcggatt
gaaaccggaaatggtcagttacatcaatgcccatggtaccagcacccctg
ctaacgatgtgacggaaacccgtgccattaaacaggcgttgggaaatcat
gcctacaatattgcggttagttctactaagtctatgaccggtcacttgtt
gggcggctccggaggtatcgaagcggtggccaccgtaatggcgatcgccg
aagataaggtaccccccaccattaatttggagaaccccgaccctgagtgt
gatttggattatgtgccggggcagagtcgggctttaatagtggatgtagc
cctatccaactcctttggttttggtggccataacgtcaccttagctttca
aaaaatatcaatagcccaccgaaaaatttcccgaaccgtgggaagatggt
agcaatttggcctgccttggcccctaccattaccgccccccggtggatat
tgacccaattattgctagtttatttttccaaacattatggtcgttgctac
ccagtccttagacgaactttctattaatgccattcgctttttagccgttg
acgccattgaaaaggccaaatctggccaccctggtttgcccatgggagcc
gctcctatggcctttaccctgtggaacaagttcatgaagttcaatcccaa
gaaccccaagtggttcaatcgggaccgctttgtgttgtccgccggccatg
gctccatgttgcagtatgccctgctctatctgctgggttatgacagtgtg
accatcgaagacattaaacagttccgtcaatgggaatcttctacccccgg
tcacccggagaattttctcactgctggagtagaagtcaccaccggcccct
tgggtcaaggcattgccaatggtgtgggtttagccctggcggaagcccat
ttggctgccacctacaacaagcctgatgccaccattgtggaccattacac
ctatgtgattctgggggatggttgcaatatggaaggtatttccggggaag
ccgcttccattgcagggcattggggtttgggtaaattaatcgcccTCTAG
Atatacg 3′

Below is the sequence of the pUC19 vector backbone and the EcoRI (gaattc) and XbaI (tctaga) sites marked in bold:

| (SEQ ID NO: 71) | |
|---|---|
| 1 | gcgcccaata cgcaaaccgc ctctccccgc gcgttggccg attcattaat gcagctggca |
| 61 | cgacaggttt cccgactgga aagcgggcag tgagcgcaac gcaattaatg tgagttagct |
| 121 | cactcattag gcaccccagg ctttacactt tatgcttccg gctcgtatgt tgtgtggaat |
| 181 | tgtgagcgga taacaatttc acacaggaaa cagctatgac catgattacg ccaagcttgc |
| 241 | atgcctgcag gtcgac tctaga ggatcccc gggtaccgag ctcgaattca ctggccgtcg |
| 301 | ttttacaacg tcgtgactgg gaaaaccctg gcgttaccca acttaatcgc cttgcagcac |
| 361 | atcccccttt cgccagctgg cgtaatagcg aagaggcccg caccgatcgc ccttcccaac |
| 421 | agttgcgcag cctgaatggc gaatggcgcc tgatgcggta ttttctcctt acgcatctgt |
| 481 | gcggtatttc acaccgcata tggtgcactc tcagtacaat ctgctctgat gccgcatagt |
| 541 | taagccagcc ccgacacceg ccaacacccg ctgacgcgcc ctgacgggct tgtctgctcc |
| 601 | cggcatccgc ttacagacaa gctgtgaccg tctccgggag ctgcatgtgt cagaggtttt |
| 661 | caccgtcatc accgaaacgc gcgagacgaa agggcctcgt gatacgccta tttttatagg |
| 721 | ttaatgtcat gataataatg gtttcttaga cgtcaggtgg cacttttcgg ggaaatgtgc |
| 781 | gcggaacccc tatttgttta tttttctaaa tacattcaaa tatgtatccg ctcatgagac |
| 841 | aataaccctg ataaatgctt caataatatt gaaaaaggaa gagtatgagt attcaacatt |
| 901 | tccgtgtcgc ccttattccc ttttttgcgg cattttgcct tcctgttttt gctcacccag |
| 961 | aaacgctggt gaaagtaaaa gatgctgaag atcagttggg tgcacgagtg ggttacatcg |
| 1021 | aactggatct caacagcggt aagatccttg agagttttcg ccccgaagaa cgttttccaa |
| 1081 | tgatgagcac ttttaaagtt ctgctatgtg gcgcggtatt atcccgtatt gacgccgggc |
| 1141 | aagagcaact cggtcgccgc atacactatt ctcagaatga cttggttgag tactcaccag |
| 1201 | tcacagaaaa gcatcttacg gatggcatga cagtaagaga attatgcagt gctgccataa |
| 1261 | ccatgagtga taacactgcg gccaacttac ttctgacaac gatcggagga ccgaaggagc |
| 1321 | taaccgcttt tttgcacaac atgggggatc atgtaactcg ccttgatcgt tgggaaccgg |
| 1381 | agctgaatga agccatacca aacgacgagc gtgacaccac gatgcctgta gcaatggcaa |
| 1441 | caacgttgcg caaactatta actggcgaac tacttactct agcttcccgg caacaattaa |
| 1501 | tagactggat ggaggcggat aaagttgcag gaccacttct gcgctcggcc cttccggctg |
| 1561 | gctggtttat tgctgataaa tctggagccg gtgagcgtgg gtctcgcggt atcattgcag |
| 1621 | cactggggcc agatggtaag ccctcccgta tcgtagttat ctacacgacg gggagtcagg |
| 1681 | caactatgga tgaacgaaat agacagatcg ctgagatagg tgcctcactg attaagcatt |
| 1741 | ggtaactgtc agaccaagtt tactcatata tactttagat tgatttaaaa cttcattttt |
| 1801 | aatttaaaag gatctaggtg aagatccttt ttgataatct catgaccaaa atcccttaac |
| 1861 | gtgagttttc gttccactga gcgtcagacc ccgtagaaaa gatcaaagga tcttcttgag |
| 1921 | atcctttttt tctgcgcgta atctgctgct tgcaaacaaa aaaaccaccg ctaccagcgg |
| 1981 | tggtttgttt gccggatcaa gagctaccaa ctctttttcc gaaggtaact ggcttcagca |
| 2041 | gagcgcagat accaaatact gttcttctag tgtagccgta gttaggccac cacttcaaga |
| 2101 | actctgtagc accgcctaca tacctcgctc tgctaatcct gttaccagtg gctgctgcca |
| 2161 | gtggcgataa gtcgtgtctt accgggttgg actcaagacg atagttaccg gataaggcgc |
| 2221 | agcggtcggg ctgaacgggg ggttcgtgca cacagcccag cttggagcga acgacctaca |
| 2281 | ccgaactgag atacctacag cgtgagctat gagaaagcgc cacgcttccc gaagggagaa |
| 2341 | aggcggacag gtatccggta agcggcaggg tcggaacagg agagcgcacg agggagcttc |
| 2401 | cagggggaaa cgcctggtat ctttatagtc ctgtcgggtt tcgccacctc tgacttgagc |
| 2461 | gtcgattttt gtgatgctcg tcaggggggc ggagcctatg gaaaaacgcc agcaacgcgg |
| 2521 | cctttttacg gttcctggcc ttttgctggc cttttgctca catgttcttt cctgcgttat |
| 2581 | cccctgattc tgtggataac cgtattaccg cctttgagtg agctgatacc gctcgccgca |
| 2641 | gccgaacgac cgagcgcagc gagtcagtga gcgaggaagc ggaaga |

The reverse-complement is shown below for ease of representing the later cloning steps:

(SEQ ID NO: 72)

tcttccgcttcctcgctcactgactcgctgcgctcggtcgttcggctgcg
gcgagcggtatcagctcactcaaaggcggtaatacggttatccacagaat
caggggataacgcaggaaagaacatgtgagcaaaaggccagcaaaaggcc
aggaaccgtaaaaaggccgcgttgctggcgtttttccataggctccgccc
ccctgacgagcatcacaaaaatcgacgctcaagtcagaggtggcgaaacc
cgacaggactataaagataccaggcgtttccccctggaagctccctcgtg
cgctctcctgttccgaccctgccgcttaccggatacctgtccgcctttct
cccttcgggaagcgtggcgctttctcatagctcacgctgtaggtatctca
gttcggtgtaggtcgttcgctccaagctgggctgtgtgcacgaacccccc
gttcagcccgaccgctgcgccttatccggtaactatcgtcttgagtccaa
cccggtaagacacgacttatcgccactggcagcagccactggtaacagga
ttagcagagcgaggtatgtaggcggtgctacagagttcttgaagtggtgg
cctaactacggctacactagaagaacagtatttggtatctgcgctctgct
gaagccagttaccttcggaaaaagagttggtagctcttgatccggcaaac
aaaccaccgctggtagcggtggtttttttgtttgcaagcagcagattacg
cgcagaaaaaaaggatctcaagaagatcctttgatcttttctacggggtc
tgacgctcagtggaacgaaaactcacgttaagggattttggtcatgagat
tatcaaaaaggatcttcacctagatccttttaaattaaaaatgaagtttt
aaatcaatctaaagtatatatgagtaaacttggtctgacagttaccaatg
cttaatcagtgaggcacctatctcagcgatctgtctatttcgttcatcca
tagttgcctgactccccgtcgtgtagataactacgatacgggagggctta
ccatctggccccagtgctgcaatgataccgcgagacccacgctcaccggc
tccagatttatcagcaataaaccagccagccggaagggccgagcgcagaa
gtggtcctgcaactttatccgcctccatccagtctattaattgttgccgg
gaagctagagtaagtagttcgccagttaatagtttgcgcaacgttgttgc
cattgctacaggcatcgtggtgtcacgctcgtcgtttggtatggcttcat
tcagctccggttcccaacgatcaaggcgagttacatgatcccccatgttg
tgcaaaaaagcggttagctccttcggtcctccgatcgttgtcagaagtaa
gttggccgcagtgttatcactcatggttatggcagcactgcataattctc
ttactgtcatgccatccgtaagatgcttttctgtgactggtgagtactca
accaagtcattctgagaatagtgtatgcggcgaccgagttgctcttgccc
ggcgtcaatacgggataataccgcgccacatagcagaactttaaaagtgc
tcatcattggaaaacgttcttcggggcgaaaactctcaaggatcttaccg
ctgttgagatccagttcgatgtaacccactcgtgcacccaactgatcttc
agcatcttttactttcaccagcgtttctgggtgagcaaaaacaggaaggc
aaaatgccgcaaaaaagggaataagggcgacacggaaatgttgaatactc
atactcttcctttttcaatattattgaagcatttatcagggttattgtct
catgagcggatacatatttgaatgtatttagaaaaataaacaaatagggg
ttccgcgcacatttccccgaaaagtgccacctgacgtctaagaaaccatt
attatcatgacattaacctataaaaataggcgtatcacgaggccctttcg
tctcgcgcgtttcggtgatgacggtgaaaacctctgacacatgcagctcc
cggagacggtcacagcttgtctgtaagcggatgccgggagcagacaagcc
cgtcagggcgcgtcagcgggtgttggcgggtgtcggggctggcttaacta
tgcggcatcagagcagattgtactgagagtgcaccatatgcggtgtgaaa
taccgcacagatgcgtaaggagaaaataccgcatcaggcgccattcgcca
ttcaggctgcgcaactgttgggaagggcgatcggtgcgggcctcttcgct
attacgccagctggcgaaagggggatgtgctgcaaggcgattaagttggg
taacgccagggttttcccagtcacgacgttgtaaaacgacggccagtgaa
ttctctagagtcgacctgcaggcatgcaagcttggcgtaatcatggtcat
agctgtttcctgtgtgaaattgttatccgctcacaattccacacaacata
cgagccggaagcataaagtgtaaagcctggggtgcctaatgagtgagcta
actcacattaattgcgttgcgctcactgcccgctttccagtcgggaaacc
tgtcgtgccagctgcattaatgaatcggccaacgcgcggggagaggcggt
ttgcgtattgggcgc

The EcoRI and XbaI sites are digested in pUC19 and in the PCR product. Below is the resulting cyanobacteria backbone vector “pScyAFT” produced after ligation of the restriction-digested DNA molecules:

(SEQ ID NO: 73)

tcttccgcttcctcgctcactgactcgctgcgctcggtcgttcggctgcg
gcgagcggtatcagctcactcaaaggcggtaatacggttatccacagaat
caggggataacgcaggaaagaacatgtgagcaaaaggccagcaaaaggcc
aggaaccgtaaaaaggccgcgttgctggcgtttttccataggctccgccc
ccctgacgagcatcacaaaaatcgacgctcaagtcagaggtggcgaaacc
cgacaggactataaagataccaggcgtttccccctggaagctccctcgtg
cgctctcctgttccgaccctgccgcttaccggatacctgtccgcctttct
cccttcgggaagcgtggcgctttctcatagctcacgctgtaggtatctca
gttcggtgtaggtcgttcgctccaagctgggctgtgtgcacgaacccccc
gttcagcccgaccgctgcgccttatccggtaactatcgtcttgagtccaa
cccggtaagacacgacttatcgccactggcagcagccactggtaacagga
ttagcagagcgaggtatgtaggcggtgctacagagttcttgaagtggtgg
cctaactacggctacactagaagaacagtatttggtatctgcgctctgct
gaagccagttaccttcggaaaaagagttggtagctcttgatccggcaaac
aaaccaccgctggtagcggtggtttttttgtttgcaagcagcagattacg
cgcagaaaaaaaggatctcaagaagatcctttgatcttttctacggggtc
tgacgctcagtggaacgaaaactcacgttaagggattttggtcatgagat
tatcaaaaaggatcttcacctagatccttttaaattaaaaatgaagtttt
aaatcaatctaaagtatatatgagtaaacttggtctgacagttaccaatg
cttaatcagtgaggcacctatctcagcgatctgtctatttcgttcatcca
tagttgcctgactccccgtcgtgtagataactacgatacgggagggctta
ccatctggccccagtgctgcaatgataccgcgagacccacgctcaccggc
tccagatttatcagcaataaaccagccagccggaagggccgagcgcagaa
gtggtcctgcaactttatccgcctccatccagtctattaattgttgccgg
gaagctagagtaagtagttcgccagttaatagtttgcgcaacgttgttgc
cattgctacaggcatcgtggtgtcacgctcgtcgtttggtatggcttcat
tcagctccggttcccaacgatcaaggcgagttacatgatcccccatgttg
tgcaaaaaagcggttagctccttcggtcctccgatcgttgtcagaagtaa
gttggccgcagtgttatcactcatggttatggcagcactgcataattctc
ttactgtcatgccatccgtaagatgcttttctgtgactggtgagtactca
accaagtcattctgagaatagtgtatgcggcgaccgagttgctcttgccc
ggcgtcaatacgggataataccgcgccacatagcagaactttaaaagtgc
tcatcattggaaaacgttcttcggggcgaaaactctcaaggatcttaccg
ctgttgagatccagttcgatgtaacccactcgtgcacccaactgatcttc
agcatcttttactttcaccagcgtttctgggtgagcaaaaacaggaaggc
aaaatgccgcaaaaaagggaataagggcgacacggaaatgttgaatactc
atactcttcctttttcaatattattgaagcatttatcagggttattgtct
catgagcggatacatatttgaatgtatttagaaaaataaacaaatagggg
ttccgcgcacatttccccgaaaagtgccacctgacgtctaagaaaccatt
attatcatgacattaacctataaaaataggcgtatcacgaggccctttcg
tctcgcgcgtttcggtgatgacggtgaaaacctctgacacatgcagctcc
cggagacggtcacagcttgtctgtaagcggatgccgggagcagacaagcc
cgtcagggcgcgtcagcgggtgttggcgggtgtcggggctggcttaacta
tgcggcatcagagcagattgtactgagagtgcaccatatgcggtgtgaaa
taccgcacagatgcgtaaggagaaaataccgcatcaggcgccattcgcca
ttcaggctgcgcaactgttgggaagggcgatcggtgcgggcctcttcgct
attacgccagctggcgaaagggggatgtgctgcaaggcgattaagttggg
taacgccagggttttcccagtcacgacgttgtaaaacgacggccagtgaa
ttccgaaaccttgctctcactaggaatgcccctgggcaacggattaccag
ccgcaacagtggcccaagcctatgttcatagcttagaaggcactatgaca
ggagaagtgctctatccgtagtaaccatatcttggtttactcttccccca
tcatggattggagataattttccagtccagaattactgataagccattgc
tgggactctaaccagtcaatttgttcttctgtttcttcaagaatttccga
caacacatcccggcttacatagtcccgttgggtttcaaagaaggcaatgc
tgttaactaaaccatccctaatgccttggttcatggtcagatcattgccc
aggatttccggtaccgtctcgccgatgagaagtttttccaaattttggag
attggggagtccttccaaaaataaaacccgctcgatcaggctatcggcct
gcttcattgccttgatggatactttatattcgtactgattaagtgcgttc
agcccccaatttttgcacatgcgagcatggagaaaatattggttaatcgc
agtaagttgtagctttaacgcttggttgagatgttgtctgacttccaggt
tgccttccatgttgttatcctctgatgtggagttttgtttgatgttgttg
tttccatttttacccattcacggtccgacgacggagttatttactgggac
agcaataaattgtttaaattgttttaatgttttacccctgggaaaattgc
ctttttctcaaaggaagtgtccctctctgaccttaaactgaaccaatatg
gctgatttgtttgtcggtgccccagttcgtttaattgcccgtccccccta
tttgaaaaccgctgatcccatgcccatgctccgtcctccggatttattgg
cgatcgccgcggagggaatggtggtagaccgtcgaccggctggctattgg
ggagtaaagtttgaccgaggcacttttctgttggaaagccagtatttgga
agtgattcggcctcaggaagaaaaaacggaagtctcggattaagaacgcc
gagtaaatgaccaagtttaatctaaaaatatggcatcaactgtaaatcgc
ctttttttagcaattttgaccatagccagcttcagccttagtggaggtta
tggatatgttcccgttcccatggcgatcgccgctgacgtcccagaactga
cagcaaaggtgcccaattatttggataaaatccaatttcctctaggggtt
atcgatgtctatggattgatgggcccagaggatggtaaacgttcccaagg
ctatgaattttgtgttgtgcccgagaaaaaaagtgaagttttggccatcg
atccctcactcacattttcgtctagccctggtcgcatcggttgcccccag
gaacaattactgtgcctaggagatacccagcaaccaaattggcaggccat
tctctttgccctggcccggttgagttacatagaaaaaatcttgccccact
ggggagaatagaagcccctatttgacaaatgtttctggccaagggacagg
ggaagcatctagtgcaagggatacctttccgttaagatggttaacgctga
acaattgagcgcattgctaaccaggcggccctgcgacagccccaagctgt
cccccgttttgctggcgatcggccgttgacccagcacgaaaactcttctt
ttatagttaaaggtattgtaatgaatcaggaaatttttgaaaaagtaaaa
aaaatcgtcgtggaacagttggaagtggatcctgacaaagtgacccccga
tgccacctttgccgaagatttaggggctgattccctcgatacagtggaat
tggtcatggccctggaagaagagtttgatattgaaattcccgatgaagtg
gcggaaaccattgataccgtgggcaaagccgttgagcatatcgaaagtaa
ataaattccggccatagccccgactccccccatagatctttggagccgag
ttctcggacggtttaagccactgtttaggactgccccaatgccggttttg
ggtttatcagtttgcccctcgggctaggccctggccccgtcgctgtatct
ttgcggagaactccaggggagtcccctccccgattctatctattaagtac
catggcaaatttggaaaagaaacgtgttgttgtaacgggattgggagcca
tcacccccatcggtaatactctccaagactattggcaaggcttaatggag
ggtcgtaacggcattggccccattacccgtttcgatgctagtgaccaagc
ctgccgttttggaggggaagtaaaggattttgatgctacccagtttcttg
accgcaaagaagctaaacggatggaccggttttgccattttgctgtttgt
gccagtcaacaggcaattaacgatgctaagttggtgattaacgaactcaa
tgccgatgaaatcggggtattgattggcacgggcattggtggtttgaaag
tactggaagatcaacaaaccattctgttggataagggtcctagccgttgc
agtccttttatgatcccgatgatgatcgccaacatggcctctgggttaac
cgccatcaacttaggggccaagggtcccaataactgtacggtgacggcct
gtgcggcgggttccaatgccattggagatgcgtttcgtttggtgcaaaat
ggctatgctaaggcaatgatttgcggtggcacggaagcggccattacccc
gctgagctatgcaggttttgcttcggcccgggctttatctttccgcaatg
atgatcccctccatgccagtcgtcccttcgataaggaccgggatggtttt
gtgatgggggaaggatcgggcattttgatcctagaagaattggaatccgc
cttggcccggggagcaaaaatttatggggaaatggtgggctatgccatga
cctgtgatgcctatcacattaccgccccagtgccggatggtcggggagcc
accagggcgatcgcctgggccttaaaagacagcggattgaaaccggaaat
ggtcagttacatcaatgcccatggtaccagcacccctgctaacgatgtga
cggaaacccgtgccattaaacaggcgttgggaaatcatgcctacaatatt
gcggttagttctactaagtctatgaccggtcacttgttgggcggctccgg
aggtatcgaagcggtggccaccgtaatggcgatcgccgaagataaggtac
cccccaccattaatttggagaaccccgaccctgagtgtgatttggattat
gtgccggggcagagtcgggctttaatagtggatgtagccctatccaactc
ctttggttttggtggccataacgtcaccttagctttcaaaaaatatcaat
agcccaccgaaaaatttcccgaaccgtgggaagatggtagcaatttggcc
tgccttggcccctaccattaccgccccccggtggatattgacccaattat
tgctagtttatttttccaaacattatggtcgttgctacccagtccttaga
cgaactttctattaatgccattcgctttttagccgttgacgccattgaaa
aggccaaatctggccaccctggtttgcccatgggagccgctcctatggcc
tttaccctgtggaacaagttcatgaagttcaatcccaagaaccccaagtg
gttcaatcgggaccgctttgtgttgtccgccggccatggctccatgttgc
agtatgccctgctctatctgctgggttatgacagtgtgaccatcgaagac
attaaacagttccgtcaatgggaatcttctacccccggtcacccggagaa
ttttctcactgctggagtagaagtcaccaccggccccttgggtcaaggca
ttgccaatggtgtgggtttagccctggcggaagcccatttggctgccacc
tacaacaagcctgatgccaccattgtggaccattacacctatgtgattct
gggggatggttgcaatatggaaggtatttccggggaagccgcttccattg
cagggcattggggtttgggtaaattaatcgccctctagagtcgacctgca
ggcatgcaagcttggcgtaatcatggtcatagctgtttcctgtgtgaaat
tgttatccgctcacaattccacacaacatacgagccggaagcataaagtg
taaagcctggggtgcctaatgagtgagctaactcacattaattgcgttgc
gctcactgcccgctttccagtcgggaaacctgtcgtgccagctgcattaa
tgaatcggccaacgcgcggggagaggcggtttgcgtattgggcgc

A unique BglII site is present between the Acp gene and the FabF gene and is used to insert a multiple cloning site. The list of restriction enzyme sequences as they appear in the multiple cloning site is BglII-BclI-EcoRV-MluI-PmeI-SpeI-BamHI and is represented by the following sequence:

(SEQ ID NO: 74)

5′ AGATCTtgatcaGATATCacgcgtGTTTAAACactagtGGATCC 3′

This oligomer is inserted into the BglII site, preserving the BglII site on one end of the multiple cloning site and destroying the BamHI and BglII sites on the other end. After non-directional ligation of the oligomer into pScyAFT, the recombinant molecule with the following orientation is selected, and is referred to as “pScyAFT-mcs”.

(SEQ ID NO: 75)

tcttccgcttcctcgctcactgactcgctgcgctcggtcgttcggctgcg
gcgagcggtatcagctcactcaaaggcggtaatacggttatccacagaat
caggggataacgcaggaaagaacatgtgagcaaaaggccagcaaaaggcc
aggaaccgtaaaaaggccgcgttgctggcgtttttccataggctccgccc
ccctgacgagcatcacaaaaatcgacgctcaagtcagaggtggcgaaacc
cgacaggactataaagataccaggcgtttccccctggaagctccctcgtg
cgctctcctgttccgaccctgccgcttaccggatacctgtccgcctttct
cccttcgggaagcgtggcgctttctcatagctcacgctgtaggtatctca
gttcggtgtaggtcgttcgctccaagctgggctgtgtgcacgaacccccc
gttcagcccgaccgctgcgccttatccggtaactatcgtcttgagtccaa
cccggtaagacacgacttatcgccactggcagcagccactggtaacagga
ttagcagagcgaggtatgtaggcggtgctacagagttcttgaagtggtgg
cctaactacggctacactagaagaacagtatttggtatctgcgctctgct
gaagccagttaccttcggaaaaagagttggtagctcttgatccggcaaac
aaaccaccgctggtagcggtggtttttttgtttgcaagcagcagattacg
cgcagaaaaaaaggatctcaagaagatcctttgatcttttctacggggtc
tgacgctcagtggaacgaaaactcacgttaagggattttggtcatgagat
tatcaaaaaggatcttcacctagatccttttaaattaaaaatgaagtttt
aaatcaatctaaagtatatatgagtaaacttggtctgacagttaccaatg
cttaatcagtgaggcacctatctcagcgatctgtctatttcgttcatcca
tagttgcctgactccccgtcgtgtagataactacgatacgggagggctta
ccatctggccccagtgctgcaatgataccgcgagacccacgctcaccggc
tccagatttatcagcaataaaccagccagccggaagggccgagcgcagaa
gtggtcctgcaactttatccgcctccatccagtctattaattgttgccgg
gaagctagagtaagtagttcgccagttaatagtttgcgcaacgttgttgc
cattgctacaggcatcgtggtgtcacgctcgtcgtttggtatggcttcat
tcagctccggttcccaacgatcaaggcgagttacatgatcccccatgttg
tgcaaaaaagcggttagctccttcggtcctccgatcgttgtcagaagtaa
gttggccgcagtgttatcactcatggttatggcagcactgcataattctc
ttactgtcatgccatccgtaagatgcttttctgtgactggtgagtactca
accaagtcattctgagaatagtgtgtgcggcgaccgagttgctcttgccc
ggcgtcaatacgggataataccgcgccacatagcagaactttaaaagtgc
tcatcattggaaaacgttcttcggggcgaaaactctcaaggatcttaccg
ctgttgagatccagttcgatgtaacccactcgtgcacccaactgatcttc
agcatcttttactttcaccagcgtttctgggtgagcaaaaacaggaaggc
aaaatgccgcaaaaaagggaataagggcgacacggaaatgttgaatactc
atactcttcctttttcaatattattgaagcatttatcagggttattgtct
catgagcggatacatatttgaatgtatttagaaaaataaacaaatagggg
ttccgcgcacatttccccgaaaagtgccacctgacgtctaagaaaccatt
attatcatgacattaacctataaaaataggcgtatcacgaggccctttcg
tctcgcgcgtttcggtgatgacggtgaaaacctctgacacatgcagctcc
cggagacggtcacagcttgtctgtaagcggatgccgggagcagacaagcc
cgtcagggcgcgtcagcgggtgttggcgggtgtcggggctggcttaacta
tgcggcatcagagcagattgtactgagagtgcaccatatgcggtgtgaaa
taccgcacagatgcgtaaggagaaaataccgcatcaggcgccattcgcca
ttcaggctgcgcaactgttgggaagggcgatcggtgcgggcctcttcgct
attacgccagctggcgaaagggggatgtgctgcaaggcgattaagttggg
taacgccagggttttcccagtcacgacgttgtaaaacgacggccagtgaa
ttccgaaaccttgctctcactaggaatgcccctgggcaacggattaccag
ccgcaacagtggcccaagcctatgttcatagcttagaaggcactatgaca
ggagaagtgctctatccgtagtaaccatatcttggtttactcttccccca
tcatggattggagataattttccagtccagaattactgataagccattgc
tgggactctaaccagtcaatttgttcttctgtttcttcaagaatttccga
caacacatcccggcttacatagtcccgttgggtttcaaagaaggcaatgc
tgttaactaaaccatccctaatgccttggttcatggtcagatcattgccc
aggatttccggtaccgtctcgccgatgagaagtttttccaaattttggag
attggggagtccttccaaaaataaaacccgctcgatcaggctatcggcct
gcttcattgccttgatggatactttatattcgtactgattaagtgcgttc
agcccccaatttttgcacatgcgagcatggagaaaatattggttaatcgc
agtaagttgtagctttaacgcttggttgagatgttgtctgacttccaggt
tgccttccatgttgttatcctctgatgtggagttttgtttgatgttgttg
tttccatttttacccattcacggtccgacgacggagttatttactgggac
agcaataaattgtttaaattgttttaatgttttacccctgggaaaattgc
ctttttctcaaaggaagtgtccctctctgaccttaaactgaaccaatatg
gctgatttgtttgtcggtgccccagttcgtttaattgcccgtccccccta
tttgaaaaccgctgatcccatgcccatgctccgtcctccggatttattgg
cgatcgccgcggagggaatggtggtagaccgtcgaccggctggctattgg
ggagtaaagtttgaccgaggcacttttctgttggaaagccagtatttgga
agtgattcggcctcaggaagaaaaaacggaagtctcggattaagaacgcc
gagtaaatgaccaagtttaatctaaaaatatggcatcaactgtaaatcgc
ctttttttagcaattttgaccatagccagcttcagccttagtggaggtta
tggatatgttcccgttcccatggcgatcgccgctgacgtcccagaactga
cagcaaaggtgcccaattatttggataaaatccaatttcctctaggggtt
atcgatgtctatggattgatgggcccagaggatggtaaacgttcccaagg
ctatgaattttgtgttgtgcccgagaaaaaaagtgaagttttggccatcg
atccctcactcacattttcgtctagccctggtcgcatcggttgcccccag
gaacaattactgtgcctaggagatacccagcaaccaaattggcaggccat
tctctttgccctggcccggttgagttacatagaaaaaatcttgccccact
ggggagaatagaagcccctatttgacaaatgtttctggccaagggacagg
ggaagcatctagtgcaagggatacctttccgttaagatggttaacgctga
acaattgagcgcattgctaaccaggcggccctgcgacagccccaagctgt
cccccgttttgctggcgatcggccgttgacccagcacgaaaactcttctt
ttatagttaaaggtattgtaatgaatcaggaaatttttgaaaaagtaaaa
aaaatcgtcgtggaacagttggaagtggatcctgacaaagtgacccccga
tgccacctttgccgaagatttaggggctgattccctcgatacagtggaat
tggtcatggccctggaagaagagtttgatattgaaattcccgatgaagtg
gcggaaaccattgataccgtgggcaaagccgttgagcatatcgaaagtaa
ataaattccggccatagccccgactccccccataGATCTtgatcaGATAT
CacgcgtGTTTAAACactagtGgatctttggagccgagttctcggacggt
ttaagccactgtttaggactgccccaatgccggttttgggtttatcagtt
tgcccctcgggctaggccctggccccgtcgctgtatctttgcggagaact
ccaggggagtcccctccccgattctatctattaagtaccatggcaaattt
ggaaaagaaacgtgttgttgtaacgggattgggagccatcacccccatcg
gtaatactctccaagactattggcaaggcttaatggagggtcgtaacggc
attggccccattacccgtttcgatgctagtgaccaagcctgccgttttgg
aggggaagtaaaggattttgatgctacccagtttcttgaccgcaaagaag
ctaaacggatggaccggttttgccattttgctgtttgtgccagtcaacag
gcaattaacgatgctaagttggtgattaacgaactcaatgccgatgaaat
cggggtattgattggcacgggcattggtggtttgaaagtactggaagatc
aacaaaccattctgttggataagggtcctagccgttgcagtccttttatg
atcccgatgatgatcgccaacatggcctctgggttaaccgccatcaactt
aggggccaagggtcccaataactgtacggtgacggcctgtgcggcgggtt
ccaatgccattggagatgcgtttcgtttggtgcaaaatggctatgctaag
gcaatgatttgcggtggcacggaagcggccattaccccgctgagctatgc
aggttttgcttcggcccgggctttatctttccgcaatgatgatcccctcc
atgccagtcgtcccttcgataaggaccgggatggttttgtgatgggggaa
ggatcgggcattttgatcctagaagaattggaatccgccttggcccgggg
agcaaaaatttatggggaaatggtgggctatgccatgacctgtgatgcct
atcacattaccgccccagtgccggatggtcggggagccaccagggcgatc
gcctgggccttaaaagacagcggattgaaaccggaaatggtcagttacat
caatgcccatggtaccagcacccctgctaacgatgtgacggaaacccgtg
ccattaaacaggcgttgggaaatcatgcctacaatattgcggttagttct
actaagtctatgaccggtcacttgttgggcggctccggaggtatcgaagc
ggtggccaccgtaatggcgatcgccgaagataaggtaccccccaccatta
atttggagaaccccgaccctgagtgtgatttggattatgtgccggggcag
agtcgggctttaatagtggatgtagccctatccaactcctttggttttgg
tggccataacgtcaccttagctttcaaaaaatatcaatagcccaccgaaa
aatttcccgaaccgtgggaagatggtagcaatttggcctgccttggcccc
taccattaccgccccccggtggatattgacccaattattgctagtttatt
tttccaaacattatggtcgttgctacccagtccttagacgaactttctat
taatgccattcgctttttagccgttgacgccattgaaaaggccaaatctg
gccaccctggtttgcccatgggagccgctcctatggcctttaccctgtgg
aacaagttcatgaagttcaatcccaagaaccccaagtggttcaatcggga
ccgctttgtgttgtccgccggccatggctccatgttgcagtatgccctgc
tctatctgctgggttatgacagtgtgaccatcgaagacattaaacagttc
cgtcaatgggaatcttctacccccggtcacccggagaattttctcactgc
tggagtagaagtcaccaccggccccttgggtcaaggcattgccaatggtg
tgggtttagccctggcggaagcccatttggctgccacctacaacaagcct
gatgccaccattgtggaccattacacctatgtgattctgggggatggttg
caatatggaaggtatttccggggaagccgcttccattgcagggcattggg
gtttgggtaaattaatcgccctctagagtcgacctgcaggcatgcaagct
tggcgtaatcatggtcatagctgtttcctgtgtgaaattgttatccgctc
acaattccacacaacatacgagccggaagcataaagtgtaaagcctgggg
tgcctaatgagtgagctaactcacattaattgcgttgcgctcactgcccg
ctttccagtcgggaaacctgtcgtgccagctgcattaatgaatcggccaa
cgcgcggggagaggcggtttgcgtattgggcgc

A selectable marker gene is then inserted into “pScyAFT-mcs”. The aph(3″)-Ia gene (GI:159885342) from Salmonella enterica subsp. chlolerasuis Tn903 provides resistance to kanamycin and neomycin. Its sequence is shown here:

(SEQ ID NO: 76)

Atgagccatattcaacgggaaacgtcttgctcgaggccgcgattaaattc
caacatggatgctgatttatatgggtataaatgggctcgcgataatgtcg
ggcaatcaggtgcgacaatctatcgattgtatgggaagcccgatgcgcca
gagttgtttctgaaacatggcaaaggtagcgttgccaatgatgttacaga
tgagatggtcagactaaactggctgacggaatttatgcctcttccgacca
tcaagcattttatccgtactcctgatgatgcatggttactcaccactgcg
atccccgggaaaacagcattccaggtattagaagaatatcctgattcagg
tgaaaatattgttgatgcgctggcagtgttcctgcgccggttgcattcga
ttcctgtttgtaattgtccttttaacagcgatcgcgtatttcgtctcgct
caggcgcaatcacgaatgaataacggtttggttgatgcgagtgattttga
tgacgagcgtaatggctggcctgttgaacaagtctggaaagaaatgcata
agcttttgccattctcaccggattcagtcgtcactcatggtgatttctca
cttgataaccttatttttgacgaggggaaattaataggttgtattgatgt
tggacgagtcggaatcgcagaccgataccaggatcttgccatcctatgga
actgcctcggtgagttttctccttcattacagaaacggctttttcaaaaa
tatggtattgataatcctgatatgaataaattgcagtttcatttgatgct
cgatgagtttttctaa

It is PCR amplified from vector pGPS5 (New England Biolabs) with primers: Forward 5′ ctataccTGATCAtaaacagtaatacaaggggtgttATG 3′ (SEQ ID NO: 77) and Reverse 5′ ccgtataACGCGTttagaaaaactcatcgagcatc 3′ (SEQ ID NO: 78) This adds a restriction endonuclease recognition sequence for BclI to the 5′ end and MluI to the 3′ end. The resulting 865 base pair product is shown below:

(SEQ ID NO: 79)

5′ctataccTGATCAtaaacagtaatacaaggggtgttATGagccatatt
caacgggaaacgtcttgctcgaggccgcgattaaattccaacatggatgc
tgatttatatgggtataaatgggctcgcgataatgtcgggcaatcaggtg
cgacaatctatcgattgtatgggaagcccgatgcgccagagttgtttctg
aaacatggcaaaggtagcgttgccaatgatgttacagatgagatggtcag
actaaactggctgacggaatttatgcctcttccgaccatcaagcatttta
tccgtactcctgatgatgcatggttactcaccactgcgatccccgggaaa
acagcattccaggtattagaagaatatcctgattcaggtgaaaatattgt
tgatgcgctggcagtgttcctgcgccggttgcattcgattcctgtttgta
attgtccttttaacagcgatcgcgtatttcgtctcgctcaggcgcaatca
cgaatgaataacggtttggttgatgcgagtgattttgatgacgagcgtaa
tggctggcctgttgaacaagtctggaaagaaatgcataagcttttgccat
tctcaccggattcagtcgtcactcatggtgatttctcacttgataacctt
atttttgacgaggggaaattaataggttgtattgatgttggacgagtcgg
aatcgcagaccgataccaggatcttgccatcctatggaactgcctcggtg
agttttctccttcattacagaaacggctttttcaaaaatatggtattgat
aatcctgatatgaataaattgcagtttcatttgatgctcgatgagttttt
ctaaACGCGTtatacgg 3′

The PCR product is digested with the enzymes and ligated into the BclI and MluI sites of pScyAFT-mcs, producing vector “pScyAFT-aphA3”. The sequence of vector pScyAFT-aphA3 is shown below:

(SEQ ID NO: 80)

tcttccgcttcctcgctcactgactcgctgcgctcggtcgttcggctgcg
gcgagcggtatcagctcactcaaaggcggtaatacggttatccacagaat
caggggataacgcaggaaagaacatgtgagcaaaaggccagcaaaaggcc
aggaaccgtaaaaaggccgcgttgctggcgtttttccataggctccgccc
ccctgaccgagcatcacaaaaatcgacgctcaagtcagaggtggcgaaac
ccgacaggactataaagataccaggcgtttccccctggaagctccctcgt
gcgctctcctgttccgaccctgccgcttaccggatacctgtccgcctttc
tcccttcgggaagcgtggcgctttctcatagctcacgctgtaggtatctc
agttcggtgtaggtcgttcgctccaagctgggctgtgtgcacgaaccccc
cgttcagcccgaccgctgcgccttatccggtaactatcgtcttgagtcca
acccggtaagacacgacttatcgccactggcagcagccactggtaacagg
attagcagagcgaggtatgtaggcggtgctacagagttcttgaagtggtg
gcctaactacggctacactagaagaacagtatttggtatctgcgctctgc
tgaagccagttaccttcggaaaaagagttggtagctcttgatccggcaaa
caaaccaccgctggtagcggtggtttttttgtttgcaagcagcagattac
gcgcagaaaaaaaggatctcaagaagatcctttgatcttttctacggggt
ctgacgctcagtggaacgaaaactcacgttaagggattttggtcatgaga
ttatcaaaaaggatcttcacctagatccttttaaattaaaaatgaagttt
taaatcaatctaaagtatatatgagtaaacttggtctgacagttaccaat
gcttaatcagtgaggcacctatctcagcgatctgtctatttcgttcatcc
atagttgcctgactccccgtcgtgtagataactacgatacgggagggctt
accatctggccccagtgctgcaatgataccgcgagacccacgctcaccgg
ctccagatttatcagcaataaaccagccagccggaagggccgagcgcaga
agtggtcctgcaactttatccgcctccatccagtctattaattgttgccg
ggaagctagagtaagtagttcgccagttaatagtttgcgcaacgttgttg
ccattgctacaggcatcgtggtgtcacgctcgtcgtttggtatggcttca
ttcagctccggttcccaacgatcaaggcgagttacatgatcccccatgtt
gtgcaaaaaagcggttagctccttcggtcctccgatcgttgtcagaagta
agttggccgcagtgttatcactcatggttatggcagcactgcataattct
cttactgtcatgccatccgtaagatgcttttctgtgactggtgagtactc
aaccaagtcattctgagaatagtgtatgcggcgaccgagttgctcttgcc
cggcgtcaatacgggataataccgcgccacatagcagaactttaaaagtg
ctcatcattggaaaacgttcttcggggcgaaaactctcaaggatcttacc
gctgttgagatccagttcgatgtaacccactcgtgcacccaactgatctt
cagcatcttttactttcaccagcgtttctgggtgagcaaaaacaggaagg
caaaatgccgcaaaaaagggaataagggcgacacggaaatgttgaatact
catactcttcctttttcaatattattgaagcatttatcagggttattgtc
tcatgagcggatacatatttgaatgtatttagaaaaataaacaaataggg
gttccgcgcacatttccccgaaaagtgccacctgacgtctaagaaaccat
tattatcatgacattaacctataaaaataggcgtatcacgaggccctttc
gtctcgcgcgtttcggtgatgacggtgaaaacctctgacacatgcagctc
ccggagacggtcacagcttgtctgtaagcggatgccgggagcagacaagc
ccgtcagggcgcgtcagcgggtgttggcgggtgtcggggctggcttaact
atgcggcatcagagcagattgtactgagagtgcaccatatgcggtgtgaa
ataccgcacagatgcgtaaggagaaaataccgcatcaggcgccattcgcc
attcaggctgcgcaactgttgggaagggcgatcggtgcgggcctcttcgc
tattacgccagctggcgaaagggggatgtgctgcaaggcgattaagttgg
gtaacgccagggttttcccagtcacgacgttgtaaaacgacggccagtga
attccgaaaccttgctctcactaggaatgcccctgggcaacggattacca
gccgcaacagtggcccaagcctatgttcatagcttagaaggcactatgac
aggagaagtgctctatccgtagtaaccatatcttggtttactcttccccc
atcatggattggagataattttccagtccagaattactgataagccattg
ctgggactctaaccagtcaatttgttcttctgtttcttcaagaatttccg
acaacacatcccggcttacatagtcccgttgggtttcaaagaaggcaatg
ctgttaactaaaccatccctaatgccttggttcatggtcagatcattgcc
caggatttccggtaccgtctcgccgatgagaagtttttccaaattttgga
gattggggagtccttccaaaaataaaacccgctcgatcaggctatcggcc
tgcttcattgccttgatggatactttatattcgtactgattaagtgcgtt
cagcccccaatttttgcacatgcgagcatggagaaaatattggttaatcg
cagtaagttgtagctttaacgcttggttgagatgttgtctgacttccagg
ttgccttccatgttgttatcctctgatgtggagttttgtttgatgttgtt
gtttccatttttacccattcacggtccgacgacggagttatttactggga
cagcaataaattgtttaaattgttttaatgttttacccctgggaaaattg
cctttttctcaaaggaagtgtccctctctgaccttaaactgaaccaatat
ggctgatttgtttgtcggtgccccagttcgtttaattgcccgtcccccct
atttgaaaaccgctgatcccatgcccatgctccgtcctccggatttattg
gcgatcgccgcggagggaatggtggtagaccgtcgaccggctggctattg
gggagtaaagtttgaccgaggcacttttctgttggaaagccagtatttgg
aagtgattcggcctcaggaagaaaaaacggaagtctcggattaagaacgc
cgagtaaatgaccaagtttaatctaaaaatatggcatcaactgtaaatcg
cctttttttagcaattttgaccatagccagcttcagccttagtggaggtt
atggatatgttcccgttcccatggcgatcgccgctgacgtcccagaactg
acagcaaaggtgcccaattatttggataaaatccaatttcctctaggggt
tatcgatgtctatggattgatgggcccagaggatggtaaacgttcccaag
gctatgaattttgtgttgtgcccgagaaaaaaagtgaagttttggccatc
gatccctcactcacattttcgtctagccctggtcgcatcggttgccccca
ggaacaattactgtgcctaggagatacccagcaaccaaattggcaggcca
ttctctttgccctggcccggttgagttacatagaaaaaatcttgccccac
tggggagaatagaagcccctatttgacaaatgtttctggccaagggacag
gggaagcatctagtgcaagggatacctttccgttaagatggttaacgctg
aacaattgagcgcattgctaaccaggcggccctgcgacagccccaagctg
tcccccgttttgctggcgatcggccgttgacccagcacgaaaactcttct
tttatagttaaaggtattgtaatgaatcaggaaatttttgaaaaagtaaa
aaaaatcgtcgtggaacagttggaagtggatcctgacaaagtgacccccg
atgccacctttgccgaagatttaggggctgattccctcgatacagtggaa
ttggtcatggccctggaagaagagtttgatattgaaattcccgatgaagt
ggcggaaaccattgataccgtgggcaaagccgttgagcatatcgaaagta
aataaattccggccatagccccgactccccccataGATCTtGATCAtaaa
cagtaatacaaggggtgttATGagccatattcaacgggaaacgtcttgct
cgaggccgcgattaaattccaacatggatgctgatttatatgggtataaa
tgggctcgcgataatgtcgggcaatcaggtgcgacaatctatcgattgta
tgggaagcccgatgcgccagagttgtttctgaaacatggcaaaggtagcg
ttgccaatgatgttacagatgagatggtcagactaaactggctgacggaa
tttatgcctcttccgaccatcaagcattttatccgtactcctgatgatgc
atggttactcaccactgcgatccccgggaaaacagcattccaggtattag
aagaatatcctgattcaggtgaaaatattgttgatgcgctggcagtgttc
ctgcgccggttgcattcgattcctgtttgtaattgtccttttaacagcga
tcgcgtatttcgtctcgctcaggcgcaatcacgaatgaataacggtttgg
ttgatgcgagtgattttgatgacgagcgtaatggctggcctgttgaacaa
gtctggaaagaaatgcataagcttttgccattctcaccggattcagtcgt
cactcatggtgatttctcacttgataaccttatttttgacgaggggaaat
taataggttgtattgatgttggacgagtcggaatcgcagaccgataccag
gatcttgccatcctatggaactgcctcggtgagttttctccttcattaca
gaaacggctttttaaaaatatggtattgataatcctgatatgaataaatt
gcagtttcatttgatgctcgatgagtttttctaaAcgcgtGTTTAAACac
tagtGgatctttggagccgagttctcggacggtttaagccactgtttagg
actgccccaatgccggttttgggtttatcagtttgcccctcgggctaggc
cctggccccgtcgctgtatctttgcggagaactccaggggagtcccctcc
ccgattctatctattaagtaccatggcaaatttggaaaagaaacgtgttg
ttgtaacgggattgggagccatcacccccatcggtaatactctccaagac
tattggcaaggcttaatggagggtcgtaacggcattggccccattacccg
tttcgatgctagtgaccaagcctgccgttttggaggggaagtaaaggatt
ttgatgctacccagtttcttgaccgcaaagaagctaaacggatggaccgg
ttttgccattttgctgtttgtgccagtcaacaggcaattaacgatgctaa
gttggtgattaacgaactcaatgccgatgaaatcggggtattgattggca
cgggcattggtggtttgaaagtactggaagatcaacaaaccattctgttg
gataagggtcctagccgttgcagtccttttatgatcccgatgatgatcgc
caacatggcctctgggttaaccgccatcaacttaggggccaagggtccca
ataactgtacggtgacggcctgtgcggcgggttccaatgccattggagat
gcgtttcgtttggtgcaaaatggctatgctaaggcaatgatttgcggtgg
cacggaagcggccattaccccgctgagctatgcaggttttgcttcggccc
gggctttatctttccgcaatgatgatcccctccatgccagtcgtcccttc
gataaggaccgggatggttttgtgatgggggaaggatcgggcattttgat
cctagaagaattggaatccgccttggcccggggagcaaaaatttatgggg
aaatggtgggctatgccatgacctgtgatgcctatcacattaccgcccca
gtgccggatggtcggggagccaccagggcgatcgcctgggccttaaaaga
cagcggattgaaaccggaaatggtcagttacatcaatgcccatggtacca
gcacccctgctaacgatgtgacggaaacccgtgccattaaacaggcgttg
ggaaatcatgcctacaatattgcggttagttctactaagtctatgaccgg
tcacttgttgggcggctccggaggtatcgaagcggtggccaccgtaatgg
cgatcgccgaagataaggtaccccccaccattaatttggagaaccccgac
cctgagtgtgatttggattatgtgccggggcagagtcgggctttaatagt
ggatgtagccctatccaactcctttggttttggtggccataacgtcacct
tagctttcaaaaaatatcaatagcccaccgaaaaatttcccgaaccgtgg
gaagatggtagcaatttggcctgccttggcccctaccattaccgcccccc
ggtggatattgacccaattattgctagtttatttttccaaacattatggt
cgttgctacccagtccttagacgaactttctattaatgccattcgctttt
tagccgttgacgccattgaaaaggccaaatctggccaccctggtttgccc
atgggagccgctcctatggcctttaccctgtggaacaagttcatgaagtt
caatcccaagaaccccaagtggttcaatcgggaccgctttgtgttgtccg
ccggccatggctccatgttgcagtatgccctgctctatctgctgggttat
gacagtgtgaccatcgaagacattaaacagttccgtcaatgggaatcttc
tacccccggtcacccggagaattttctcactgctggagtagaagtcacca
ccggccccttgggtcaaggcattgccaatggtgtgggtttagccctggcg
gaagcccatttggctgccacctacaacaagcctgatgccaccattgtgga
ccattacacctatgtgattctgggggatggttgcaatatggaaggtattt
ccggggaagccgcttccattgcagggcattggggtttgggtaaattaatc
gccctctagagtcgacctgcaggcatgcaagcttggcgtaatcatggtca
tagctgtttcctgtgtgaaattgttatccgctcacaattccacacaacat
acgagccggaagcataaagtgtaaagcctggggtgcctaatgagtgagct
aactcacattaattgcgttgcgctcactgccgctttccagtcgggaaacc
tgtcgtgccagctgcattaatgaatcggccaacgcgcggggagaggcggt
ttgcgtattgggcgc

### EXAMPLE 23

In an exemplified embodiment of this invention, one or more algal or cyanobacterial lines are identified as showing a statistical difference in fluorescence, isoprenoid flux, or fatty acid content compared to the wild-type; identification of any line showing no statistical difference despite transgene expression of IPPI or accD under various promoters is also a measurable embodiment. Dunaliella and Tetraselmis are ideal candidates for characterization and selection by flow cytometry and by High Pressure Liquid Chromatography (HPLC) due to the non-aggregating nature of cultures and their pigmentation, respectively. Flow cytometry is used to select for cells with altered isoprenoid flux, or other measurable altered fluorescence or growth characteristics, resulting from payload uptake, nucleic acid integration, or transgene expression. Cultures can be preserved with 0.5% paraformaldehyde, then frozen to −20° C. Thawed samples were analyzed on a Beckman-Coulter Altra flow cytometer equipped with a Harvard Apparatus syringe pump for quantitative sample delivery. Cells are excited using a water-cooled 488 nm argon ion laser. Populations were distinguished based on their light scatter (forward and 90 degree side) as described in previous Examples. Resulting files are analyzed using FlowJo (Tree Star, Inc.). Cell lines of interest are then bulked up for further characterization, such as for pigments, nucleic acid content or fatty acid content.

HPLC is used for analysis of IPPI lines, to assess pigmented isoprenoids likely affected by the expression of this rate-limiting enzyme. Cells are filtered through Whatman GF/F filters (2.5 cm), hand-ground, and extracted for 24 hr (0° C.) in acetone. Pigment analyses are performed in triplicate using a ThermoSeparation UV2000 detector (□=436 nm). Eluting pigments are identified by comparison of retention times with those of pure standards and algal extracts of known pigment composition. The numbers reported are pigment concentrations in ng/L; data are then converted to amount per million cells, based on total cell number in each sample. Means analysis by Student's t test is done to reveal any significant increase in intermediate and endpoint carotenoids relative to chlorophyll a, and indicate possible functionality of the inserted genes for increasing isoprenoid flux. Cell lines of interest are bulked up for further characterization by transgene detection and by fatty acid content. For the latter, nucleic acids are prepared any number of standard protocols. Briefly, cells are centrifuged at 1000×g for 10 min. To the cell pellet, 500 uL of lysis buffer (20 mM Tris-HCl, 200 mM Na-EDTA, 15 mM NaCl, 1% SDS)+3 uL of RNAase are added and incubated at 65° C. for 20 min. This was mixed intermittently. After centrifuge at 10,000×g for 5 min the supernatant is transferred to a new centrifuge tube. Extraction of DNA is done by adding equal volumes of phenol-chloroform-isoamyl alcohol (24:24:1), followed by centrifugation. The aqueous layer is then transferred to a new 1.5 mL Eppendorf tube, and the DNA is precipitated with 2 vol of 100% ethanol. After precipitation, the DNA pellet is washed with 70% ethanol, and dissolved in TE buffer (10 mM Tris-HCl, 1 mM EDTA, pH 8.0). The concentration of the DNA is ascertained spectrophotometrically. Primers are designed for within inserted genes and within chloroplast sequences as is known in the art, and PCR conditions for each primer set is determined using standard practices. Amplified DNA can be sequenced to verify presence of target nucleic acids.

Lipid content and composition is assessed by fatty acid methyl-ester (FAME) analysis, using any number of protocols as is known in the art. In one exemplification, cell pellets are stored under liquid nitrogen prior to analysis. Lipids are extracted using a Dionex Accelerated Solvent Extractor (ASE; Dionex, Salt Lake City) system. The lipid fraction is evaporated and the residue is heated at 90° C. for 2 hr with 1 mL of 5% (w/w) HCl-methanol to obtain fatty acid methyl esters in the presence of C19:0 as an internal standard. The methanol solution is extracted twice with 2 mL n-hexane. Gas chromatography is performed with a HP 6890 GC/MS equipped with a DB5 fused-silica capillary column (0.32 μm internal diameter×60 m, J&W Co.). The following oven temperature program provides a baseline separation of a diverse suite of fatty acid methyl esters: 50° C. (1 min hold); 50-180° C. (20° C./min); 180-280° C. (2° C./min); 280-320° C. (10° C./min); and 320° C. (10 min hold). Fatty acid methyl esters are identified on the basis of retention times, co-injection analysis using authentic standards, and MS analysis of eluting peaks.

In another exemplification, lipid content is measured by extraction of trans-esterified or non-trans-esterified oil from Tetraselmis and Dunaliella. To begin, 60 L of algal cells are harvested using a concentrator to reduce the liquid to 3 L. The volume can be further reduced by centrifugation at 5000 rpm for 15-30 min, forming a 1200 mL pellet. The cell pellet is lyophilized for 2 days, yielding the following weights: Dunaliella spp −14.21 g dry weight, 45 g wet weight; Tetraselmis spp.-48.45 g dry weight, 50 g wet weight. These were stored at −20° C. in 50 mL tubes. For extraction, lyophilized biomass weighing 15.39 g for Tetraselmis and 14.2 g for Dunaliella are employed. To the lyophilized biomass, 1140 mL of the corresponding extraction system in a conical flask is carried on for 1 h in nitrogen atmosphere with constant agitation (300:600:240 ml of Cl₃CH/MeOH/H₂O, 1:2:0.8, vol/vol/vol, monophasic). The mixture is then filtered through glass filters (100-160 μm bore). The residue is washed with 570 mL of the extraction system, and this filtrate is added to the first one. The mixture is made biphasic by the addition of 450 mL chloroform and 450 mL water, giving an upper hydromethanolic layer and a lower layer of chloroform in which lipids are present. This is shaken well and left for an hour to form a clear biphasic layer. The lower chloroform layer that has the lipids is collected and excess chloroform is evaporated using a rotary evaporator for 2 hr until droplets of chloroform form. The remaining lipids in the hydrophilic phases, as well as other lipids, are extracted with 100 mL chloroform. The total volume is reduced to 10 mL in a vacuum evaporator at 30° C. The extract is further subjected to a speed vacuum overnight to remove excess water and chloroform. For Tetraselmis spp. CCMP908, for example, 2.735 g oil was obtained from 48.45 g dry weight for an approximate 18% oil content for the cells. For Dunaliella spp, 4.4154 g oil was obtained from 14.21 g dry weight for an approximate 31% oil content for cells, without accounting for salt residues that can be removed by 0.5 M ammonium bicarbonate. The methodology can be scaled down, for example to allow analyses with mg quantities.

### EXAMPLE 24

In an exemplified embodiment of this invention, one or more algal or cyanobacterial lines identified to be of interest for scale-up and field testing are taken from flask culture into carboys then into outdoor photobioreactors. Ponds or raceways are an additional option. All field production is subject to appropriate permitting as necessary. Lab scale-up can occur, as one example, from culture plates to flask culture volumes of 25 mL, 125 mL, 500 mL, 1 L, then into carboy volumes of 2.5 L, 12.5 L, 20 L, 62.5 L (for example using multiple carboys), which are bubbled for air exchange and mixing, prior to seeding of bioreactors such as the Varicon Aquaflow BioFence System (Worcestershire, Great Britain) at 200 L, 400 L, 600 L, 1000 L, and 2400 L volumes. Other options can be systems from IGV/B. Braun Biotech Inc. (Allentown, Pa.) and BioKing BV (Gravenpolder, The Netherlands) or vertical tubular reactors of approximately 400 L volumes employed commercially such as at Cyanotech Corp. (Kona, Hi.). Culture can proceed under increasing light conditions so as to harden-off the algae for outdoor light conditions. This can be from 100, 200, 300, 400, 600 uE/m2-sec indoors to 400, 600, 1200 to 2000 uE/m2-sec outdoors using shading when necessary. For example, a 1:20 dilution can be used such that 1 L of log-phase culture is used to inoculate 20 L of medium in one or multiple carboys. Culture of algae in photobioreactors, degassing, pH monitoring, dewatering for biomass harvest, and oil extraction proceeds as described (Christi, Y. Biotechnology Advances 25: 294-306; 2007). Photobioreactors have higher density cultures and thus can be combined for biphasic production with a raceway pond as the final 1- to 2-day grow-out phase under oil induction conditions such as nitrogen stress. Alternatively, production of biomass for biofuels using raceways can proceed as is known in the art (Sheehan J, et al., National Renewable Energy Laboratory, Golden Colo., Report NREL/TP-580-24190: 145-204; 1998). Production can proceed under varied conditions of pH and carbon dioxide supplementation.

Depending on the species, one or more algal or cyanobacterial lines can be grown heterotrophically or mixotrophically in stirred tanks or fermentors such as for Nannochloropsis, Tetraselmis, Chlorella, as described for the latter by the Yaeyama Shokusan Co., Ltd. and in Li Xiufeng, et al., Biotechnology and Bioengineering 98: 764-771; 2007, or for the facultative heterotrophic cyanobacterium Synechocystis sp. PCC 6803. In yet another embodiment, the hydrocarbon yields of one or more of the above organisms can be modulated by culture under nitrogen deplete rather than replete conditions, as is known in the art for Dunaliella, Haematococcus, and other microalgae. In yet another embodiment, the hydrocarbon composition and yields can be altered by pH or carbon dioxide levels, as is known in the art for Dunaliella.

### EXAMPLE 25

This example illustrates a nucleic acid which encodes a gene that participates in fatty acid biosynthesis, beta ketoacyl ACP synthase (KAS).

Fatty acid synthesis begins in the chloroplast of higher plants and in bacteria with the condensation of acetyl-CoA and malonyl-CoA, catalyzed by KASIII, also known as FabH (Tsay et al., J. Biol. Chem. 267:6807-6814; 1992). Elongation of the hydrocarbon chain is accomplished by KASI (FabB) and KASII (FabF) catalyzing the condensation of additional malonyl-ACP units. KASI predominantly catalyzes the elongation to unsaturated 16:0 palmitoyl-ACP and KASII promotes elongation of 16:1 to 18:1, which cannot be performed by KASI (Subrahmanyam and Cronan, J. Bacteriol. 180:4596-4602; 1998).

One example of use of this family of enzymes is to create a preferential-length hydrocarbon molecule. A host cell is modified by means described in the previous Examples to express the Cuphea KASII to preferentially form C8 and C10 hydrocarbon chains. This is accompanied by the transformation with, and expression of an acyl-ACP thioesterase that prefers medium-chain hydrocarbons as taught above.

Below is a list of several KAS enzymes that may be used in various embodiments described herein. Additional KAS enzymes that can be used may be identified from other species using a degenerate PCR approach similar to that outlined in Examples 10, 11 and 12.

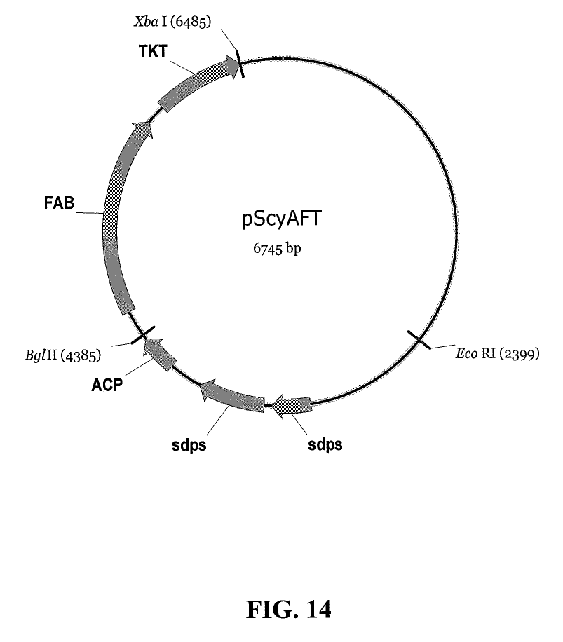

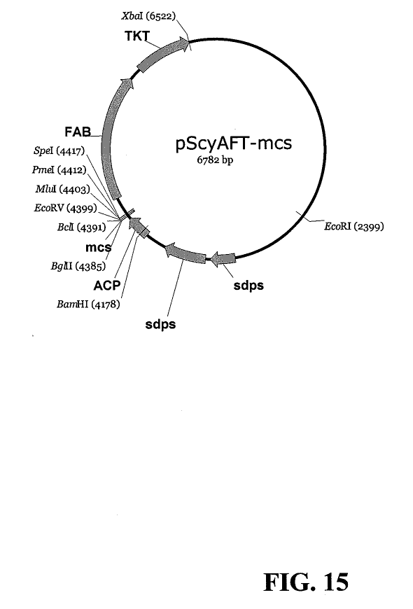

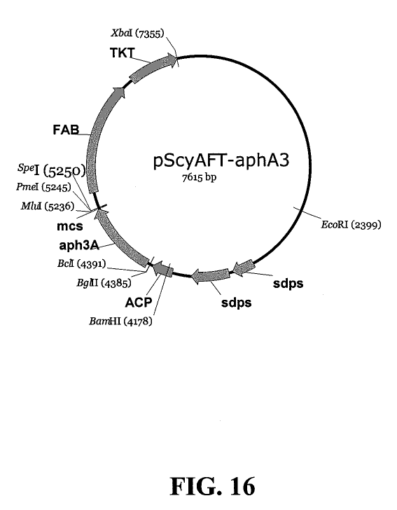

Following is the sequence of Synechocystis sp. PCC 6803 beta keto-acyl-ACP synthase (accession number BAA000022.2; GI47118304; region 820102 . . . 821352). This sequence is found in, for example, the vectors shown in FIGS. 14, 15 and 16 (pScyAFT; pScyAFT-mcs; pScyAFT-aphA3):

| (SEQ ID NO: 85) | |
|---|---|
| 1 | ctattgatat tttttgaaag ctaaggtgac gttatggcca ccaaaaccaa aggagttgga |
| 61 | tagggctaca tccactatta aagcccgact ctgccccggc acataatcca aatcacactc |
| 121 | agggtcgggg ttctccaaat taatggtggg gggtacctta tcttcggcga tcgccattac |
| 181 | ggtggccacc gcttcgatac ctccggagcc gcccaacaag tgaccggtca tagacttagt |
| 241 | agaactaacc gcaatattgt aggcatgatt tcccaacgcc tgtttaatgg cacgggtttc |
| 301 | cgtcacatcg ttagcagggg tgctggtacc atgggcattg atgtaactga ccatttccgg |
| 361 | tttcaatccg ctgtctttta aggcccaggc gatcgccctg gtggctcccc gaccatccgg |
| 421 | cactggggcg gtaatgtgat aggcatcaca ggtcatggca tagcccacca tttccccata |
| 481 | aatttttgct ccccgggcca aggcggattc caattcttct aggatcaaaa tgcccgatcc |
| 541 | ttcccccatc acaaaaccat cccggtcctt atcgaaggga cgactggcat ggaggggatc |
| 601 | atcattgcgg aaagataaag cccgggccga agcaaaacct gcatagctca gcggggtaat |
| 661 | ggccgcttcc gtgccaccgc aaatcattgc cttagcatag ccattttgca ccaaacgaaa |
| 721 | cgcatctcca atggcattgg aacccgccgc acaggccgtc accgtacagt tattgggacc |
| 781 | cttggcccct aagttgatgg cggttaaccc agaggccatg ttggcgatca tcatcgggat |
| 841 | cataaaagga ctgcaacggc taggaccctt atccaacaga atggtttgtt gatettecag |
| 901 | tactttcaaa ccaccaatgc ccgtgccaat caataccccg atttcatcgg cattgagttc |
| 961 | gttaatcacc aacttagcat cgttaattgc ctgttgactg gcacaaacag caaaatggca |
| 1021 | aaaccggtcc atccgtttag cttctttgcg gtcaagaaac tgggtagcat caaaatcctt |
| 1081 | tacttcccct ccaaaacggc aggcttggtc actagcatcg aaacgggtaa tggggccaat |
| 1141 | gccgttacga ccctccatta agccttgcca atagtcttgg agagtattac cgatgggggt |
| 1201 | gatggctccc aatcccgtta caacaacacg tttcttttcc aaatttgcca t |

Following is the sequence of Phaeodactylum tricornutum keto-acyl-CoA synthase (PtKAS) accession number AY746358:

| (SEQ ID NO: 86) | |
|---|---|
| 1 | atggctccgc aacaacgaaa ccccgtactc aatgaagacg gaaacacggg gatgcgacgg |
| 61 | gtggactccg aggcttccga catgagtgaa ctcggcaacg atacacgagc gcaagactat |
| 121 | cgcatccgta agagttcctt gattggaatg atcgactggg ggcacgttat ggtgtcccat |
| 181 | cttcccttgc taatggtcgt gggtatcctg acgctggtgg cgcagattgt gcaccaggtt |
| 241 | gttattgaac tcggtctgca aaacattgac tggtccgtgc agaccgtgtc gaccatctgt |
| 301 | cacgccatca aggagctctt tcgcgatttg tacgcttcca ttatggaaag ccgcggcttt |
| 361 | gacttattct cccccgccgt caaaaccacc gccctcctgt tgttcctcgg cgcctggtgg |
| 421 | atgagacgca agagtcccgt ctatcttttg tcctttgcaa ccttcaaggc cccggattct |
| 481 | tggaaaatgt cgcacgcaca gattgtggaa attatgcgcc gtcaagggtg cttttccgaa |
| 541 | gactcgctcg aattcatggg caaaattctg gcgcgctcgg gtaccggcca agccacggct |
| 601 | tggcctccgg gcataacccg ctgtctacag gacgaaaaca ccaaagccga tcggtccatc |
| 661 | gaagcggcac gccgcgaagc cgaaatcgtc atctttgacg tcgtcgaaaa ggctctccaa |
| 721 | aaagcccgcg tccggcccca agacattgac attctcatta tcaactgcag tttgttcagc |
| 781 | ccaactccct cgttgtgcgc catggtactg tcccactttg gcatgcgcag cgacgttgcc |
| 841 | accttcaatt tgtccggcat gggctgttcc gcctcgctca ttagcatcga tctcgccaaa |
| 901 | tccctcttgg gcacccggcc gaatagcaag gccctcgtgg tgagtacgga aatcatcacg |
| 961 | cccgccttgt accacggcag cgaccggggc tttttgatcc aaaacacact cttccgctgt |
| 1021 | ggcggagccg ctatggtgtt gagcaattcc tggtacgacg gtcgccgcgc ctggtacaag |
| 1081 | ctgctacaca cggtccgggt gcagggcacc aacgaagccg ccgtctcgtg cgtctacgaa |
| 1141 | accgaagacg cccagggaca tcagggtgta cgcttgagta aggatatcgt caaggtggcg |
| 1201 | ggcaaatgca tggaaaagaa ctttaccgtt ttgggtccgt ccgtgctgcc gctgacggag |
| 1261 | caagccaagg tggtggtgtc gattgccgcc cggtttgttc tgaaaaagtt cgaagggtac |
| 1321 | acgaaacgca aggtaccgtc gattcggccg tacgtgccgg atttcaaacg cggcatcgac |
| 1381 | cacttttgta tccacgccgg gggacgtgcc gtgattgacg gtatcgaaaa gaatatgcag |
| 1441 | ctgcaaatgt accacaccga ggcgtcgcgt atgacgctac tgaattacgg caacacgagc |
| 1501 | agcagcagta tctggtacga gttggagtac attcaggacc agcaaaagac gaatccgctg |
| 1561 | aaaaagggcg accgggtatt gcaagtggcg ttcgggtccg gcttcaagtg cacgtccggg |
| 1621 | gtgtggctca agctctaa |

Following is the nucleotide sequence of the Arabidopsis thaliana KASIII enzyme (accession number AY091275; GI:20258996):

| (SEQ ID NO: 87) | |
|---|---|
| 1 | atggctaatg catctgggtt cttcactcat ccttcaattc ctaacttgcg aagcagaatc |
| 61 | catgttccgg ttagagtttc tggatctggg ttttgcgttt ccaatcgatt ctctaagagg |
| 121 | gttttgtgct ctagcgtcag ctccgtcgat aaggatgctt cgtcttctcc ttctcaatat |
| 181 | caacgaccca ggctagtgcc gagtggctgc aaattgattg gatgtggatc agcagttcca |
| 241 | agtcttctga tttctaatga tgatctcgct aaaatagttg atactaatga tgaatggatt |
| 301 | gctactcgta ctggtattcg caaccgtcga gttgtctcag gcaaagatag cttggttggc |
| 361 | ttagcagtag aagcagcaac caaagctctt gaaatggctg aggttgttcc tgaagatatt |
| 421 | gacttagtct tgatgtgtac ttccactcct gatgatctat ttggtgctgc tccacagatt |
| 481 | caaaaggcac ttggttgcac aaagaaccca ttggcttatg atatcacagc tgcttgtagt |
| 541 | ggatttgttt tgggtctagt ttcagctgct tgtcatataa ggggaggcgg ttttaagaac |
| 601 | gttttagtga tcggagctga ttctttgtct cggtttgttg attggacgga tagagggact |
| 661 | tgcattctat ttggagatgc tgctggtgct gtggttgttc aggcttgtga tattgaagat |
| 721 | gatggtttgt tcagttttga tgtgcacagc gatggggatg gtcgaagaca tttgaatgct |
| 781 | tctgttaaag aatcccaaaa cgatggtgaa tcaagctcca atggctcggt gtttggagac |
| 841 | tttccaccaa aacaatcttc atattcttgt attcagatga atggaaaaga ggtctttcgc |
| 901 | tttgctgtca aatgtgttcc tcaatctatt gaatctgctt tacaaaaagc tggtcttcct |
| 961 | gcttctgcca tcgactggct cctcctccac caggcgaacc agagaataat agactctgtg |
| 1021 | gctacaaggc tgcatttccc accagagaga gtcatatcga atttggctaa ttatggtaac |
| 1081 | acgagcgctg cttcgattcc gctggctctt gatgaggcag tgagaagcgg aaaagttaaa |
| 1141 | ocaggacata ccatagcgac atccggtttt ggagccggtt taacgtgggg atcagcaatt |
| 1201 | atgcgatgga ggtgaatggc taagtccaac aatgtaagtt aacttc |

Following is the nucleotide sequence of the Arabidopsis thaliana KASI enzyme (accession number NM_{—}123998.2; GI:30694933):

| (SEQ ID NO: 88) | |
|---|---|
| 1 | gaacataagc tcttttcgca aaacacacat cacacaccat tttcacaaca tcgtacttat |
| 61 | cgccttcctc tctctctcaa tacctctctc aatttctgga tccaccatgc aagctcttca |
| 121 | atcttcatct ctccgtgctt ctcctccaaa cccacttcgc ttaccatcaa atcgtcaatc |
| 181 | acatoageta attaccaatg cgagaccttt gcgaagacaa caacgttcct tcatctccgc |
| 241 | atcagcatcc actgtctccg ctcctaaacg cgaaacagat ccgaagaaac gagttgtcat |
| 301 | tactggtatg ggtctcgtct ctgtgtttgg taacgatgtt gatgcttact acgagaaatt |
| 361 | gttgtctggt gagagtggaa tcagtttgat tgatcgtttc gatgcttcca agttccctac |
| 421 | tcgattcggt ggtcagatcc gtgggtttag ctctgaaggt tatattgatg gcaagaatga |
| 481 | gcgtaggctt gatgattgtt tgaaatattg cattgttgct ggtaaaaaag ctcttgaaag |
| 541 | tgccaatctt ggtggtgata agcttaacac gattgataag aggaaagctg gagtactagt |
| 601 | tgggactgga atgggaggtt taactgtgtt ttcagaaggt gttcagaatt tgattgagaa |
| 661 | gggtcatagg aggattagtc cattttttat accttatgct ataacaaata tgggttctgc |
| 721 | tttgttggcg attgatcttg gtcttatggg tcctaactat tcgatttcaa ctgcttgtgc |
| 781 | tacttcgaat tactgctttt acgctgctgc gaatcacatt cgtcgtggtg aagctgatat |
| 841 | gatgattgct ggtgggactg aggctgctat tattcctatt gggttgggag gttttgttgc |
| 901 | ttgtagggca ttgtcccaga gaaatgatga ccctcaaact gcttccaggc cgtgggataa |
| 961 | agcaagagat gggtttgtta tgggtgaagg agctggtgtt ctggtgatgg aaagcttgga |
| 1021 | acatgcaatg aaacgtggtg ctccaattgt agcagaatat cttggaggtg ctgttaattg |
| 1081 | tgatgctcac catatgactg atccaagagc tgatggtctt ggggtttctt catgcattga |
| 1141 | aagatgcctg gaagatgctg gtgtatcacc tgaggaggta aattacatca atgcacatgc |
| 1201 | aacttccact cttgctggtg atcttgctga gattaatgcc attaaaaagg tattcaagag |
| 1261 | cacttcaggg atcaaaatca acgccaccaa gtctatgata ggtcactgcc tcggtgcagc |
| 1321 | tggaggtcta gaagccatcg ccaccgtgaa ggctatcaac actggatggc tgcatccttc |
| 1381 | catcaaccaa tttaacccag aacaagctgt ggactttgac acggtcccaa acgagaagaa |
| 1441 | gcaacacgag gttgatgttg ccatatcaaa ctcgttcggg ttcggtggac acaactcggt |
| 1501 | agtcgccttc tctgccttca aaccctgatt tcttcatacc ttttagattc tctgccctat |
| 1561 | cggttactat catcatccat caccaccact tgcagcttct tggttcacaa gttggagctc |
| 1621 | ttcctctggc cttttgcggt tctttcattc cccgtttctt acggttgctg agatttcaga |
| 1681 | ttttgtttgt tctctctctt gtctgcggaa tgttgtgtat cttagttcgt tccatatttg |
| 1741 | cgtaatttat aaaaacagaa actgagagaa tcttgtagta acggtgttat tgtcagaata |
| 1801 | atccaattag gggattctca tcttttattt ctcaacaatt cttgtcgtgt ttttacattc |
| 1861 | gaagaaatta gatttatact g |

What is claimed is:

1. A method for producing a gene product of interest in marine algae comprising:
transforming a marine alga with a vector comprising a first chloroplast genome sequence, a second chloroplast genome sequence and a gene encoding a product of interest, wherein said gene is flanked by the first and second chloroplast genome sequences; and
culturing said marine alga, thereby producing the product of interest.

2. The method of claim 1, additionally comprising collecting the product of interest from the marine alga.

3. The method of claim 1, wherein said first and second chloroplast genome sequences each comprises at least 300 contiguous base pairs of SEQ ID NO: 4.

4. The method of claim 1, wherein said product of interest is selected from the group consisting of IPP isomerase, acetyl-coA synthetase, pyruvate dehydrogenase, pyruvate decarboxylase, acetyl-coA carboxylase, α-carboxyltransferase, β-carboxyltransferase, biotin carboxylase, biotin carboxyl carrier protein and acyl-ACP thioesterase, beta ketoacyl-ACP synthase, FatB, and a protein that participates in fatty acid biosynthesis via the pyruvate dehydrogenase complex.

5. The method of claim 4, wherein said acetyl-coA carboxylase is selected from the group consisting of biotin carboxylase (BC), biotin carboxyl carrier protein (BCCP), α-carboxyltransferase (α-CT) and β-carboxyltransferase (β-CT).

6. The method of claim 4, wherein said protein that participates in fatty acid biosynthesis via the pyruvate dehydrogenase complex is selected from Pyruvate dehydrogenase E1α, Pyruvate dehydrogenase E1β, dihydrolipoamide acetyltransferase, dihydrolipoamide dehydrogenase, and pyruvate decarboxylase.

7. The method of claim 1, wherein said product of interest is beta ketoacyl ACP synthase and expression of the beta ketoacyl ACP synthase modifies fatty acid chain length.

8. The method of claim 1, wherein said vector comprises a second gene encoding a product of interest.

9. The method of claim 8, wherein the first and second genes are expressed coordinately in a polycistronic operon.

10. A plastid nucleic acid sequence for plastome recombination in unicellular bioprocess marine algae comprising SEQ ID NO: 4.

11. A vector for targeted integration in the plastid genome of a unicellular bioprocess marine algae comprising a first segment of chloroplast genome sequence and a second segment of chloroplast genome sequence.

12. The vector of claim 11, wherein said first and second segments of chloroplast genome sequence each comprise at least 300 contiguous base pairs of SEQ ID NO: 4.

13. The vector of claim 11, further comprising a gene of interest located between the first and second segments of chloroplast genome sequence.

14. The vector of claim 13, wherein said gene of interest does not interfere with production of a gene product encoded by the first and second segments.

15. The vector of claim 13, wherein the gene of interest is operably linked to a transcriptional promoter from an operon of the targeted integration site.

16. A unicellular bioprocess marine alga transformed with a vector comprising:
a first segment of chloroplast genome sequence;
a second segment of chloroplast genome sequence; and
a gene of interest located between the first and second segments of chloroplast genome sequence.

17. The unicellular bioprocess marine alga of claim 16, wherein said bioprocess marine alga is of the species Dunaliella or Tetraselmis.

18. A method of integrating a gene of interest into the plastid genome of a unicellular bioprocess marine alga comprising transforming a unicellular bioprocess marine alga with a vector comprising a first segment of chloroplast genome sequence, a second segment of chloroplast genome sequence, and a gene of interest, wherein said gene of interest is located between the first and second segments of chloroplast genome sequence.

19. The method of claim 18, wherein said transforming is carried out using magnetophoresis, electroporation, or a particle inflow gun.

20. The method of claim 19, wherein said magnetophoresis is moving pole magnetophoresis.

21. The method of claim 18, wherein said gene of interest is introduced into the plastid genome.

22. The method of claim 18, wherein said gene of interest encodes a selectable marker.

23. The method of claim 18, wherein said gene of interest encodes a molecule selected from the group consisting of IPP isomerase, acetyl-coA synthetase, pyruvate dehydrogenase, pyruvate decarboxylase, acetyl-coA carboxylase, α-carboxyltransferase, β-carboxyltransferase, biotin carboxylase, biotin carboxyl carrier protein and acyl-ACP thioesterase, beta ketoacyl-ACP synthase, FatB, and a protein that participates in fatty acid biosynthesis via the pyruvate dehydrogenase complex.

24. A method for isolation of a plastid nucleic acid from unicellular bioprocess marine algae for determination of contiguous plastid genome sequences comprising:
passing the algae through a French press;
isolating the chloroplasts using density gradient centrifugation;
lysing the isolated chloroplasts; and
isolating the plastid nucleic acid by density gradient centrifugation.

25. The method of claim 24, wherein said plastid nucleic acid is a high molecular weight plastid nucleic acid.

26. The method of claim 24, wherein said unicellular bioprocess marine algae is selected from the group consisting of Dunaliella and Tetraselmis.

27. The method of claim 24, wherein the algae is Dunaliella, and is passed through the French press for about 2 minutes at a pressure of about 700 psi.

28. The method of claim 24, wherein the algae is Tetraselmis, and is passed through the French press for about 2 minutes at a pressure of 3000 to 5000 psi.

29. A method for producing a gene product of interest in cyanobacteria comprising:
transforming a cyanobacteria with a vector comprising a first clustered orthologous group sequence, a second clustered orthologous group sequence and a gene encoding a product of interest, wherein said gene is flanked by the first and second clustered orthologous group sequences; and
culturing said cyanobacteria to produce the gene product.

30. The method of claim 29, additionally comprising collecting the gene product from the cyanobacteria.

31. The method of claim 29, wherein said first and second clustered orthologous group sequences each comprises at least 300 contiguous base pairs of SEQ ID NO: 70.

32. The method of claim 29, wherein said gene product is selected from the group consisting of IPP isomerase, acetyl-coA synthetase, pyruvate dehydrogenase, pyruvate decarboxylase, acetyl-coA carboxylase, α-carboxyltransferase, β-carboxyltransferase, biotin carboxylase, biotin carboxyl carrier protein and acyl-ACP thioesterase, beta ketoacyl-ACP synthase, FatB, and a protein that participates in fatty acid biosynthesis via the pyruvate dehydrogenase complex.

33. The method of claim 29, wherein the vector comprises two or more genes encoding products of interest.

34. The method of claim 33, wherein the two or more genes are expressed coordinately in a polycistronic operon.

35. A vector for targeted integration in the genome of a cyanobacterium comprising:
a first segment of clustered orthologous group sequence, and
a second segment of clustered orthologous group sequence.

36. The vector of claim 35, wherein said first and second segments of clustered orthologous group sequence each comprise at least 300 contiguous base pairs of SEQ ID NO: 70.

37. The vector of claim 35, further comprising a gene of interest located between the first and second segments of clustered orthologous group sequence.

38. The vector of claim 37, wherein said gene of interest does not interfere with production of a gene product encoded by the first and second segments.

39. The vector of claim 37, wherein the gene of interest is operably linked to a transcriptional promoter from an operon of the targeted integration site.

40. A cyanobacterium transformed with a vector comprising a first segment of clustered orthologous group sequence, a second segment of clustered orthologous group sequence, and a gene of interest located between the first and second segments of clustered orthologous group sequence.

41. The cyanobacterium of claim 40, wherein said cyanobacteria is of the species Synechocystis or Synechococcus.

42. A method of integrating a gene of interest into a clustered orthologous group of a cyanobacteria genome comprising transforming a cyanobacteria with a vector comprising a first segment of clustered orthologous group sequence, a second segment of clustered orthologous group sequence, and a gene of interest, wherein said gene of interest is located between the first and second segments.

43. The method of claim 42, wherein said transforming is carried out using prokaryotic conjugation or passive direct DNA uptake.

44. The method of claim 42, wherein said gene of interest encodes a molecule selected from the group consisting of IPP isomerase, acetyl-coA synthetase, pyruvate dehydrogenase, pyruvate decarboxylase, acetyl-coA carboxylase, α-carboxyltransferase, β-carboxyltransferase, biotin carboxylase, biotin carboxyl carrier protein and acyl-ACP thioesterase, beta ketoacyl-ACP synthase, FatB, and a protein that participates in fatty acid biosynthesis via the pyruvate dehydrogenase complex.
